SEQUENCE LISTING (52 sequences)

SEQ ID NO: 1 - protein, 260 aa, Homo Sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 2 - protein, 260 aa, Artificial Sequence
  mutant form of the CAII protein (TS1)
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSHDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVSKFRKLNFNGEGEPEEPMVDNWRPAQPLKNRQIKASFK

SEQ ID NO: 3 - protein, 260 aa, Artificial Sequence
  mutant form of the CAII protein (TS3)
MSHHWGFGKHNGPEHWHKDFPIAKGERQSPVDIDTHTAKYDPSLKPLSVSYDQATSLRILNNGHAFQVEFDDSQDKAVLKGGPLDGTYRLIQFHFHWGSHDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVSKFRKLNFNGEGEPEEPMVDNWRPAQPLKNRQIKASFK

SEQ ID NO: 4 - DNA, 3449 bp, Chlamydomonas reinhardtii
agttgtagggcccttgcattaacgaaggttaggcatcaggcggaggcgcctgaactatttcaacgactgaagaccggtcgctcattccttgcgcattgctgctttggtagatgcgtgttaccgcatagagcagcctgcttgcaattcagtttttgatctctaagatagagcagcgcctgcaaaaggcgcagacgctttcgtcagatgcttcctgcgtcgcttcaacgcaaggccgctgccgttggcggtcgcggccccaccaaccagagtcgcgtggcagttcgcgtctctgctcagccgaaggaagctcctcccgcctcgacacccatcgttgaggacccggagagcaagttccgccgctatggcaagcatttcggcggcattcacaagctgagcatggattggcttgatagcgttcctcgcgtgcgcgtgcgcaccaaggactctcgccagctggacgatatgttggagctggcagtgctcaacgagcgccttgcgggtcgcttggagccctggcaggctcgtcagaagcttgagtacctccgtaagcggcggaagaactgggagcgcattttcgagtacgtgacgcgtcaggatgcggccgcgaccctggccatgatcgaggaggcaaatcgcaaggtggaggagtcgctgagcgaggaggcacgcgagaagactgctgtaggcgacctccgagaccagctggagtcgctgcgcgcgcaggtggcgcaggcgcaggagcgccttgctatgacgcagtcgcgcgtggagcagaacctacagcgcgtgaatgagctgaaggcggaggcgaccacgctagagcgcatgcgcaaggcctcggacctggacatcaaggagcgcgagcgcatcgccatctccactgtcgccgccaagggaccggcctcgagcagcagcagcgccgccgccgtcagcgcccccgccacgtcggccacgctgacggtggagcgccccgccgccaccacggtgacgcaggaggtgccgtccaccagctacggcacccccgtggaccgcgcgccgcgccgcagcaaggcggccatccggcgcagccgcgggctggaaagcagcatggagattgaggagggcctgcgcaacttctggtaccccgctgagttctcagcgcgcttgccgaaggacacgctggtgccctttgagctgtttggcgagccgtgggtgatgttccgtgatgagaaggggcagccctcctgcatccgcgacgagtgcgcacaccgcggctgcccgctcagcctgggcaaggtggtggagggacaggtcatgtgcccctaccacggctgggagttcaacggcgacggcgcctgcaccaagatgccctccacgcccttctgccgcaatgtgggcgttgccgcgctgccttgcgcggagaaggatggcttcatctgggtctggcccggcgacggcctgccagcggagacgctgccggacttcgcccagccgccagagggctttctgatccacgcggagatcatggtggatgtgcctgtggagcacggcctgctgattgagaacctgctggacctggcgcacgcgccgttcacgcacaccagcaccttcgcgcgcggctggcctgtgcccgacttcgtcaagttccatgccaacaaggcgctctcgggcttctgggacccctaccccatcgacatggccttccagccgccctgcatgacgctgtccaccatcggcctggcgcaacccggcaagattatgcgcggcgtgaccgccagccagtgcaagaaccacctgcaccagctgcacgtgtgcatgccctccaagaagggccacacgcggctgctgtaccgcatgagcctggacttcctgccctggatgcgccacgtgcccttcatcgaccgcatctggaagcaggtggcggcgcaggtgctgggcgaggacctggtgctggtgctgggccagcaggaccgcatgctgcgcggcggcagcaactggtccaaccccgcgccctacgacaagctggcggtgcgctaccgccgctggcgcaacggcgtaaacgccgaggtcgcacgcgtgcgcgccggcgagccaccgtccaaccccgtggcaatgagcgcgggcgagatgttctcggtggacgaggatgacatggacaactagaagccacgtggcgtggattggcgagcggaggtggcaggagcgagcatgggcgtggtggaggatagagcggcgagggcagctagggccgtggtgcaggcggcggggtgtacatggctgaggtgggcagcggcaggcgcagcaaacgcggctagagaccgaggccaattcatgcaggagcccgtcgagagcgtgttagggtcagcttcagggtattacgggtgcatgagtgtggtaggtacaggtggttaggcgtccatgtttgagccactgcgtgtgcaaatagtgcttggacagccgtgcgccaggtgcgtaatagtatgtccatggatcactgaacaatgagaagatacaatctgtggactcatacatagtgcggggtttgttatcagatgtcgggcggccgcgcagtgtgtgtcgctggaaggtatcggcaatgtgcgaggaagtgtacactgttggtgcctgtagctagtgcgcttggtgcgtcgcgtgtgtgcaagtcatggttcctggcgggagtcagcgtgcaatggaccacttcatccgctgcccggatgttaaggtacgtgtgcgttgaggatgagagtctggttggagagccagtggcagaggggcaaggccctttgctactttgtgatcgcgtgctcatcgttgctattgttttttgccggcgtaagcggcgtggtggaggacgcaacgtgtgctgcagctgggtgttgagatcgagggacccgaagcacacggctcagaagaacgttttcatccagcctggagaggtgtgcgtgtgctgcggtcaatgagtttgcgctggcgtccagaacgactcttggggatgcgttgttgagacgtagggttagggtttggtatgaagtgcaccgaaagagcagcagtgagtggcaagtgcccctttctgcgctgttcggcccctgcaagttgaagtagttcttggatgcagtcccaacccgggcatgcggtcggtgctggtgtatcaaacaatctggagttttggtgtccggccatgggtgtcgctgtgtgtgttcatttcggggaggctgagttccaacggcccctaggccgccgcttgggggtctccgctgtgtaccattgaatcggtctgcagactgggttccgtacccaattaattttgtttcgcggtctttcataacgcgtaagaacccgcgtcggaagagtggaaatggttggtggtgagaaggagcggctcgtcagtacggaggtgttgacggagctccagtgagaaagtacagcgaaatactgtaacgctagctgctgaaaaaaaaaaaaaaaaa

SEQ ID NO: 5 - DNA, 1974 bp, Volvox carteri f. nagariensis
atgcttccagcacaaagacagtgcaggacgtccgcctgccaaggcaggggcattataagcaagaggactatccgtgctgactttaaagtccatgcgtcagtatcacagcagccttcttcagacaagcctgagcaacaggctgtaccgtctatcgtcgaggaccctgaagcgaagtttcggcgttatggcaagcatttcggtggtatccataagctaaatctggattggctggaggcagttccgcgtgtgcgtgttcggaccaaagattcacggcagctcgacgagctgttggagctggcagtgctcaatgagcgccttgcgggacgcttggagccttggcaggcacgccagaagcttgagtatctgcgtaagcgccggaagaactgggagcgcatctttgagtacgtcactaagcaggacgctgctgccacgctagccatgatcgaggaggccaaccgaaaggtggaggaagccttgtcggaagaggcacgcgagcgaacagcagtgggagatttgcgggagcagcttcaagtcctgcaacgccaggtgcaggaggcgcaggagcggcttcagctcacgcaagcacgtgtggagcagaacctgaaccgcgtgaatgagctgaaggcagaggcggtcggcctggagcggatgcgaaacggaaggatgggtggcgatcgcaagaaggagctccaggtggcggcgccagtcgctgtcactgccgcggcgtcggcggcacgtcctgctgtttctgctacggcagtggcggaatcagtccccgcggccatcgtgacagtggagccccctaccaggagctatacccccaatggctcgtccgatggcacgtcggttgtcgccccaccaggtcgtcgcagcaaggtagccatccgacggggtcgcggtctggagagcagcttggacttcgagccaggccttcgcaacttttggtaccctgcggagttttcagcgaagctgggtcaggacacgctggttcccttcgagctgtttggggagccctgggtcctgttccgcgacgagaaggggcagcccgcttgcatcaaggacgaatgcgcacatcgggcctgcccgttgtcgcttggaaaggtggtagaggggcaggttgtgtgcgcgtaccacggctgggagttcaacggcgatggccactgcaccaagatgccctccacgccgcattgccgcaacgtgggggtatcggcgctgccctgcgctgagaaggatggcttcatctgggtgtggcctggagacggactcccggcgcagacgctccccgacttcgcacgcccaccggagggctttcaagtgcacgctgagattatggtggacgtgccggtggagcatggcctgctcatggagaaccttttggatctggcgcatgcgccattcacccacaccacaacttttgcgcgcggctggcccgtgcctgacttcgtcaagttccacaccaacaaattactatcgggatactgggacccctaccccatcgacatggctttccagccgccttgcatggttctgtccacgattggcttggcgcaacctggcaagattatgcgcggcgtgacggcatcgcaatgcaagaaccatctgcaccagctccatgtgtgcatgccgtcgaagaagggccacacgcggctgctgtaccgcatgagcctagacttcctgccgtggatgcgctacgtgccgtttattgacaaggtctggaagaatgttgcgggccaggtgttgggcgaggacctggtgctggtgctggggcaacaggatcgtttgctgcgcggcgggaacacctggtcgaacccggcgccgtacgacaagctggcggtacgataccgccgctggcgcaactcggtcagtcccgatggcgctggccttgacggcccggcgccactgaacccagtggcgatgagcgccggggagatgttttcaattgatgaagatgagcaggatccgcggatgcagtga

SEQ ID NO: 6 - DNA, 1869 bp, Dunaliella salina
tcaacaggggttggggccatgcaatcaaagctcttggggcttcaagacgagattagtgaggcaagggacaagctgcgtacctcagaggcaagggtggcacaaaacctcaagcgtgtggatgagttgaaggctgaggcggcttccttggagcgcatgcgcctggccagcagctcaagcactgacagcacagtcagcattgccagcagggggggcgcagctgtggctgcaaccacgagcgtaccggaccatgtggagagggaagggatccagagcagggtgcggggcagtggcatggcctcaacaagctacccctcccatgtacctcagccgagccaggcagtgagacggggccctaaaccgaaggacagcaggcgactgagaagcagcctggagctggaagacggcctgcgcaacttctggtacccgaccgagtttgcgaagaagctggagccgggcatgatggtgccctttgacttgttcggcgtgccgtgggtgctgttccgagatgagcacagcgcccccacctgcatcaaggactcctgcgcgcaccgcgcatgcccgctgtcactgggcaaggtcatcaacggccacgtgcagtgcccctaccatggctgggagtttgacgggagcggcgcgtgcaccaagatgcccagcacgcgcatgtgccatggcgtgggcgtggccgcgctgccgtgcgtggagaaggacggctttgtgtgggtgtggcctggggatgggcccccacctgacctgccgccggacttcacagccccccctgcaggctatgacgtgcacgcagagatcatggtggatgtgcctgtggagcacggcctgctgatggagaacttacttgatctggcccacgcgcccttcacccacaccaccacctttgcgcggggctggcccatcccagaggctgtgcgcttccatgccaccaagatgctggcaggtgactgggacccctaccccatcagcatgtcttttaaccccccctgcattgcgctgtcaaccatcgggctgtcgcagcctggcaagatcatgcgcggctacaaggcagaggagtgcaagcgccacctacaccagctgcacgtgtgcatgccctccaaggagggccacacgcgcctgctgtaccgcatgagccttgacttctggggctgggctaagcacgtgccatttgtggatgtgctgtggaagaagattgctggccaggtgctgggtgaggacctggtgctggtgctggggcagcaggctcgcatgattggcggcgacgacacctggtgcacgcccatgccgtacgacaagctggctgtgcggtaccggaggtggcggaacatggtggctgatggtgagtacgaggaggggtctcggaatcgctgcacaagccaatatgacagctggccagatgtttgactcccacgatgatgaggatctgtatgagcatcagcgccatgatgaggggaacctgcagggccagcaaagcagcgtttttgctgcaaggaagtgagggcattcatcctaggtttttgcttgagcagaaggagaggcttataggatggtagaattgattgtaaaattttgtaacatgcttggtggttcaatggttcctgtacttgatgacttgtagaatttttcccgtcgagggtgttcacactgttaagtgctatgttggcggtgactgaggatgcataattgcgctgtcccaccatgcatactgttgccagttttaaacggatttcatgttgtctctccagttttgatggattgctggatggtttgttttggtctcccctttaatttctttaatttgccctactaaatgggctctcagtagaacatgtggttggaaatctgtaaggttcaagaacattt

SEQ ID NO: 7 - DNA, 894 bp, Nephroselmis pyriformis
tgcggtggagttcacttcgcgcttggggaaggacatcatggttccgtttgagtgcttcgaggagtcctgggtactcttccgcgacgaggacggcaaggcgggctgcatcaaggacgagtgcgcgcaccgcgcttgcccgctctcgctcggcacggtggagaacggccaggcgacgtgcgcgtaccacggctggcagttcagcactgggggggagtgcaccaagatcccgtcggtcggcgcgcggggctgctcgggcgtgggcgtgcgcgccatgcccaccgtggagcaagatggcatgatctggatctggcccggggacgagaagcccgccgagcacatcccgtccaaggaggtgctgccgcccgcgggccacaccctccacgcggagatagtgctggacgtgcccgtggagcacggcctgctgctggagaacctcctggacctggcgcacgcgcccttcacccacacgtccacgttcgccaagggctgggcggtcccggaactcgtcaagttctccacggacaaggtgcgcgcgctcgggggcgcgtgggaaccttaccccatcgacatgagcttcgagccgccctgcatggtgctgtccaccatcgggctcgcgcagccgggcaaggtagacgcgggcgtgcgcgcgtccgagtgcgagaagcacctgcaccagctgcacgtgtgcatgccctcgggcgcggggaagacgcgcctgctgtaccgcatgcacctcgacttcatgccgttcctcaaatacgtgccgggcatgcacctggtgtgggaggccatggccaaccaggtgctgggggaggacctgaggctggtgctggggcagcaggacaggctgcagaggggcggggacgtgtggagcaaccccatggagtacgacaa

SEQ ID NO: 8 - DNA, 1379 bp, Mesostigma viride
  misc_feature (1224)..(1224): n is a, c, g, or t
gacgaggacggccgcgtggcgtgcctgcgggatgagtgcgcgcaccgtgcatgccccctgtcactgggcacggtggagaacgggcacgcgacctgcccctaccatggctggcagtacgacacggacggcaagtgcacaaagatgccgcagacgcggctgcgcgcgcaggtgcgcgtgtccaccctgcccgtgcgcgagcacgacggcatgatctgggtgtacccagggtccaacgagccgcccgagcacctgccgtcgttcctgccccccagcaacttcacggtgcacgccgagttggtgctggaggtgcccatcgagcacgggctgatgatcgagaacctgctggacctggcacacgcgcccttcacgcacaccgagacctttgccaagggatggtcggtcccggactctgtcaacttcaaggtcgccgcgcagtcgctggcggggcattgggagccgtaccccatcagcatgaagtttgagccgccgtgcatgacgatctcggaaatcgggctggccaagcccgggcagttggaggccggcaagttcagtggcgagtgcaagcagcacctgcaccagctgcacgtgtgcatgcccgcgggggagggccgcacgcgcatcctctaccgcatgtgcctcgactttgcgcactgggtcaagtacatacccggaatccagaatgtgtggtcgggcatggcgacgcaggtgcttggggaggacctgcggctggtggaggggcagcaggatcgcatgctgcgcggcgcggacatctggtacaacccggtcgcctatgacaagctgggcgtgcggtaccgcagctggcgccgcgcggtcgagcgcaacacgcgcagccggttcatcgggggccaggagaagcttgcgcccgagggtagagactagtgagcaaaaggggtgactgctccactgtaccttcatggccgagcagccagctggtacaggcctgacaccgtggcaagcctgcacttgggccatgcagcgggttaaggttgaggcttctgatggcaacccttgtccggtctattgtacaaaacgaggaacggagaacatggctccattgcaactgtgagatgttgaggatgcatgctgctacaaggtgccagcaaggtctgtcacagggatgctccagcatgaccaatgggtgccattgcttgaaatggatatgtgctaacagggggggatttactctttgctgccccagtgtananatcatggccaggatgatacattcatcnccaatctgcagggtacntgtgaaanaacctgntggnnttgcatgccttatccnttccnantganaananttttgntgaggggcncttncngcttnttaccnaaaaannncttgccnnaaaaaaaaa

SEQ ID NO: 9 - protein, 247 aa, Chlamydomonas reinhardtii
MGEQLRQQGTVKWFNATKGFGFITPGGGGEDLFVHQTNINSEGFRSLREGEVVEFEVEAGPDGRSKAVNVTGPGGAAPEGAPRNFRGGGRGRGRARGARGGYAAAYGYPQMAPVYPGYYFFPADPTGRGRGRGGRGGAMPAMQGVMPGVAYPGMPMGGVGMEPTGEPSGLQVVVHNLPWSCQWQQLKDHFKEWRVERADVVYDAWGRSRGFGTVRFTTKEDAATACDKLNNSQIDGRTISVRLDRFA

SEQ ID NO: 10 - protein, 226 aa, Chlamydomonas incerta
MGEQLRQQGTVKWFNATKGFGFITPGGGGEDLFVHQTNINSEGFRSLREGEAVEFEVEAGPDGRSKAVNVTGPAGAAPEGAPRNFRGGGRGRGRARGARGGYAAAYGYPQMAPVYPGYYFFPADPTGRGRGRGGRGGAMPGMQGVMPGVAYPGMPMGGVGMEATGDPSGLQVVVHNLPWSCQWQQLKDHFKEWRVERADVVYDAWGRSRGFGTVRFTTKEDAAMAC

SEQ ID NO: 11 - protein, 242 aa, Volvox carteri f. nagariensis
MGEQLRQRGTVKWFNATKGFGFITPEGGGEDFFVHQTNINSDGFRSLREGEAVEFEVEAGPDGRSKAVSVSGPGGSAPEGAPRNFRGGGRGRGRARGARGAYAAYGYPQMPPMYPGYYFFPADPTGRGRGRGRGGMPIQGMIQGMPYPGIPIPGGLEPTGEPSGLQVVVHNLPWSCQWQQLKDHFKEWRVERADVVYDAWGRSRGFGTVRFATKEDAAQACEKMNNSQIDGRTISVRLDRFE

SEQ ID NO: 12 - protein, 178 aa, Physcomitrella patens subsp. Patens
AKETGKVKWFNSSKGFGFITPDKGGEDLFVHQTSIHAEGFRSLREGEVVEFQVESSEDGRTKALAVTGPGGAFVQGASYRRDGYGGPGRGAGEGGGRGTVGGAGRGRGRGGRGVGGFVGERSGAAGGERTCYNCGEGGHIARECQNESTGNARQGGGGGGGNRSCYTCGEAGHLARDC

SEQ ID NO: 13 - protein, 444 aa, Zea mays
MAAAARQRGTVKWFNDTKGFGFISPEDGSEDLFVHQSSIKSEGFRSLAEGEEVEFSVSEGDDGRTKAVDVTGPDGSSASGSRLLHDGAWRPFCIFTSTRQPEQHRGSGSDRHDGGDYNHPKPQAIAAGAHSLLLTRACLSSKSPPPSLAVGLLSVLAQRTGPTPGTTGSAASLSGSSPISLGFNPTSFLPFLQTARWLPCSDLATSSSSAPSSPPRSLAPSAPPKKALIGASTGSTGIATSSGAGAAMSRSNWLSRWVSSCSDDAKTAFAAVTVPLLYGSSLAEPKSIPSKSMYPTFDVGDRILAEKVSYIFRDPEISDIVIFRAPPGLQVYGYSSGDVFIKRVVAKGGDYVEVRDGKLFVNGVVQDEDFVLEPHNYEMEPVLVPEGYVFVLGDNRNNSFDSHNWGPLPVRNIVGRSILRYWPPSKINDTIYEPDVSRLTVPSS

SEQ ID NO: 14 - protein, 197 aa, Oryza sativa Japonica Group
MASERVKGTVKWFDATKGFGFITPDDGGEDLFVHQSSLKSDGYRSLNDGDVVEFSVGSGNDGRTKAVDVTAPGGGALTGGSRPSGGGDRGYGGGGGGGRYGGDRGYGGGGGGYGGGDRGYGGGGGYGGGGGGGSRACYKCGEEGHMARDCSQGGGGGGGYGGGGGGYRGGGGGGGGGGCYNCGETGHIARECPSKTY

SEQ ID NO: 15 - protein, 139 aa, Chlorella variabilis
MAAAKATGTVKWGYGFITPDSGGEDLFVHQTAIVSEGFRSLREGEPVEFFVETSDDGRQKAVNVTGPNGAAPEGAPRRQFDDGYGAGGGGGSYGGGFGGGGGGGRRGGGRGGGGYGGGGYGGGYDQGGYGGQPPIACNM

SEQ ID NO: 16 - protein, 182 aa, Selaginella moellendorffi
MASPADAKRTGKVKWFNVTKGFGFITPDDGSEELFVHQSAIFAEGFRSLREGEIVEFSVEQGEDQRMRAADVTGPDGSHVQGAPSSFGSRGGGGGGGRGGRGRAGGGDNPIVCYNCNEAGHVSRDCKYQQEGGGGGGGGGGGRGPPSGRRGGGAGGGSGGGGRGCFTCGAQGHISRDCPSNY

SEQ ID NO: 17 - protein, 208 aa, Vitis vinifera
MAQERSTGVVRWFSDQKGFGFITPNEGGEDLFVHQSSIKSDGFRSLGEGETVEFQIVLGEDGRTKAVDVTGPDGSSVQGSKRDNYGGGGGGGIASEEIMAAAAAVVVEEAEAEVVIPAVAVAVVITVVIMGTWLGIALWKAAALVGSVVAEVEAVEGLVAVAVDATTVDRKGILLENALTLTHRDEGKRGVIVYILFFPASSKIFFPV

SEQ ID NO: 18 - protein, 231 aa, Triticum aestivum
MGERVKGTVKWFNVTKGFGFISPDDGGEDLFVHQSAIKSDGYRSLNENDAVEFEIITGDDGRTKASDVTAPGGGALSGGSRPGEGGGDRGGRGGYGGGGGGYGGGGGGYGGGGGGYGGGGGGYGGGGYGGGGGGGRGCYKCGEDGHISRDCPQGGGGGGGYGGGGYGGGGGGGRECYKCGEEGHISRDCPQGGGGGGYGGGGGRGGGGGGGGCFSCGESGHFSRECPNKAH

SEQ ID NO: 19 - protein, 135 aa, Cryptosporidium parvum Iowa II
EKPIKLVKMPLSGVCKWFDSTKGFGFITPDDGSEDIFVHQQNIKVEGFRSLAQDERVEYEIETDDKGRRKAVNVSGPNGAPVKGDRRRGRGRGRGRGMRGRGRGGRGRGFYQNQNQSQPQSQQQPVSTQSQPVAH

SEQ ID NO: 20 - protein, 301 aa, Arabidopsis thaliana
MAMEDQSAARSIGKVSWFSDGKGYGFITPDDGGEELFVHQSSIVSDGFRSLTLGESVEYEIALGSDGKTKAIEVTAPGGGSLNKKENSSRGSGGNCFNCGEVGHMAKDCDGGSGGKSFGGGGGRRSGGEGECYMCGDVGHFARDCRQSGGGNSGGGGGGGRPCYSCGEVGHLAKDCRGGSGGNRYGGGGGRGSGGDGCYMCGGVGHFARDCRQNGGGNVGGGGSTCYTCGGVGHIAKVCTSKIPSGGGGGGRACYECGGTGHLARDCDRRGSGSSGGGGGSNKCFICGKEGHFARECTSVA

SEQ ID NO: 21 - protein, 243 aa, Artificial Sequence
  exemplary fluorescent protein, Katushka 9-5
MGEDSELISENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRKLERIKEADKETYVEQHEMAVARYCDLPSKLGHSNPQRSTVWY

SEQ ID NO: 22 - protein, 234 aa, Artificial Sequence
  exemplary fluorescent protein, Kat650-21
MGEDSELISENMHMKLYMEGTVNGHHFKCTSEGEGKPYEGTQTAKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRKLERIKEADKETYVEQHEMAVARYCDLPSKLGHS

SEQ ID NO: 23 - protein, 234 aa, Artificial Sequence
  exemplary fluorescent protein, Kat670-23
MGEDSELISENMHTKLYMEGTVNGHHFKCTSEGEGKPYEGTQTCKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEANTEMLYPADSGLRGHNQMALKLVGGGYLHCSLKTTYRSKKPAKNLKMPGFYFVDRKLERIKEADKETYVEQHEMAVARYCDLPSKLGHS

SEQ ID NO: 24 - protein, 234 aa, Artificial Sequence
  exemplary fluorescent protein, KatX1
MGEDSELISENMHTKEYMEGTVNGHHFKCTSEGEGKPYEGTQTCKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEANTEMLYPADSGLRGHNQMALKLVGGGYLHCSLKTTYRSKKPAKNLKMPGFYFVDRKLERIKEADKETYVEQHEMAVARYCDLPSKLGHS

SEQ ID NO: 25 - protein, 234 aa, Artificial Sequence
  exemplary fluorescent protein, KatX2
MGEDSELISENMHSKEYMEGTVNGHHFKCTSEGEGKPYEGTQTAKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRKLERIKEADKETYVEQHEMAVARYCDLPSKLGHS

SEQ ID NO: 26 - protein, 234 aa, Artificial Sequence
  exemplary fluorescent protein, Katusha9-5A
MGEDSELISENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHAQMALKLVGGGYLHCSLKTTYRSKKPAKNLKMPGFYFVDRKLERIKEADKETYVEQHEMAVARYCDLPSKLGHS

SEQ ID NO: 27 - DNA, 30 bp, Artificial Sequence
  Synthetic primer
gctttcgtcatatgcttcctgcgtcgcttc

SEQ ID NO: 28 - DNA, 32 bp, Artificial Sequence
  Synthetic primer
ctctggatccgtctgtgtaaatgtgatgaagc

SEQ ID NO: 29 - DNA, 29 bp, Artificial Sequence
  Synthetic primer
gacgaattcgtcagatgcttcctgcgtcg

SEQ ID NO: 30 - DNA, 30 bp, Artificial Sequence
  Synthetic primer
ctctagatctgtcgcctccgccttcagctc

SEQ ID NO: 31 - DNA, 26 bp, Artificial Sequence
  Synthetic primer
gtatcaatattgttgcgttcgggcac

SEQ ID NO: 32 - DNA, 25 bp, Artificial Sequence
  Synthetic primer
ggagcgcagccaaaccaggatgatg

SEQ ID NO: 33 - DNA, 24 bp, Artificial Sequence
  Synthetic primer
gtccccaccaccctccacaacacg

SEQ ID NO: 34 - DNA, 21 bp, Artificial Sequence
  Synthetic primer
gcaagtacaccattggcgagc

SEQ ID NO: 35 - DNA, 18 bp, Artificial Sequence
  Synthetic primer
cctttgcacagcgcacac

SEQ ID NO: 36 - DNA, 19 bp, Artificial Sequence
  Synthetic primer
gacttcctgccctggatgc

SEQ ID NO: 37 - DNA, 19 bp, Artificial Sequence
  Synthetic primer
gggttggaccagttgctgc

SEQ ID NO: 38 - DNA, 54 bp, Artificial Sequence
  Synthetic primer
atcttcatatgggccagacccccgcagggcttcctgcgtcgcttcaacgcaagg

SEQ ID NO: 39 - DNA, 37 bp, Artificial Sequence
  Synthetic primer
tagaatctagacragttgtccatgtcatcctcgtcca

SEQ ID NO: 40 - DNA, 13 bp, Artificial Sequence
  Synthetic primer
gccagacccccgc

SEQ ID NO: 41 - DNA, 29 bp, Artificial Sequence
  Synthetic primer
gctttcgtcatatgcttctgcgtcgcttc

SEQ ID NO: 42 - DNA, 32 bp, Artificial Sequence; identical to SEQ ID NO: 28
  Synthetic primer

SEQ ID NO: 43 - DNA, 29 bp, Artificial Sequence
  Synthetic primer
gacgaattcgtcagatgcttcctgcgtcg

SEQ ID NO: 44 - DNA, 30 bp, Artificial Sequence; identical to SEQ ID NO: 30
  Synthetic primer

SEQ ID NO: 45 - DNA, 26 bp, Artificial Sequence
  Synthetic primer
cagtcctgtagcttcatacaaacata

SEQ ID NO: 46 - DNA, 23 bp, Artificial Sequence
  Synthetic primer
gatcctcctgtggctaattgacc

SEQ ID NO: 47 - DNA, 30 bp, Artificial Sequence
  Synthetic primer
atcttcatatgcttcctgcgtcgcttcaac

SEQ ID NO: 48 - DNA, 37 bp, Artificial Sequence
  Synthetic primer
atcttcatarggggcaaacaccggcgggccttcctgc

SEQ ID NO: 49 - DNA, 22 bp, Artificial Sequence
  Synthetic primer
gttaggtgtttgcgctcttgac

SEQ ID NO: 50 - DNA, 21 bp, Artificial Sequence
  Synthetic primer
ggcgagtgagcatattcgtcc

SEQ ID NO: 51 - DNA, 13 bp, Artificial Sequence
  Synthetic primer
ggcaaacaccggc

SEQ ID NO: 52 - DNA, 26 bp, Artificial Sequence
  Synthetic primer
atcagttgcgtgcgccttgccaaacc

(19) United States
(12) Patent Application Publication
(10) Pub. No.: US 2014/0242676 A1
(43) Pub. Date: Aug. 28, 2014

(54) ARTIFICIAL LEAF-LIKE MICROPHOTOBIOREACTOR AND METHODS FOR MAKING THE SAME

(71) Applicant: Los Alamos National Security, LLC, Los Alamos, NM (US)

(72) Inventors: Amr I. Abdel-Fattah, Los Alamos, NM (US); Jennifer Ann Hollingsworth, Los Alamos, NM (US); Peng He, Los Alamos, NM (US); Richard Thomas Sayre, Los Alamos, NM (US); Suzanne Zoe Fisher, Los Alamos, NM (US)

(73) Assignee: Los Alamos National Security, LLC

(21) Appl. No.: 14/170,400

(22) Filed: Jan. 31, 2014

Related U.S. Application Data
(60) Provisional application No. 61/759,714, filed on Feb. 1, 2013.

(51) Int. Cl. C12N 1/12

(52) U.S. Cl. CPC C12N 1/12
USPC 435/257.1

(57) ABSTRACT

Described herein are algae carbon capture systems and biomass production systems, and more specifically, algal based microphotobioreactors (μPBRs) comprising a biocompatible polymer (e.g., hydrogel) containing algae, inorganic carbon, light-frequency shifting agents (e.g., quantum dots and/or dyes of fluorescent proteins) and methods for making such μPBRs.

### CROSS REFERENCE TO RELATED APPLICATION

Applicant claims the benefit of the earlier filing date of U.S. Provisional Application No. 61/759,714 filed on Feb. 1, 2013, the entire disclosure of which is incorporated herein by reference.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

The present disclosure relates generally to algae carbon capture systems and biomass production systems, and more specifically, to algal based microphotobioreactors (μPBRs) comprising a biocompatible polymer (e.g., hydrogel) containing algae, inorganic carbon, light-frequency shifting agents (e.g., quantum dots and/or dyes of fluorescent proteins) and methods for making such μPBRs.

### BACKGROUND

The booming global population, combined with rising industrialization and modernization generates increasing demands for energy, most of which comes from fossil fuels. Increasing greenhouse gas (GHG) emissions are accelerating climate change at a pace that has global environmental and security implications. To mitigate domestic energy demands and their environmental impacts, it is necessary to seek alternative energy sources that reduce or ameliorate carbon emissions. The potential for reductions in GHG emissions (environment), reduced fuel prices (economics), and reduction in dependency on foreign oil (national security) have driven increased scientific, public, political and commercial interests in biofuels. However, a number of limitations impede the advancement and scale-up of current biomass/biofuel production systems, including: (1) low efficiency (5-6%) of solar energy conversion into biomass, (2) high water (350 gal. H₂O)/gal oil) and nutrient demands (CO₂, N and P), (3) substantial harvesting energy costs (40% of total), (4) high environmental dependency, and (5) biocontainment constraints.

The two basic approaches to cultivating algae for biomass and biofuel production are the open pond system and enclosed photobioreactors. However, these systems, at present, suffer from most of the limitations mentioned above.

The open pond system uses shallow ponds (about 15 to 20 cm deep) to cultivate massive amounts of algae under conditions that are nearly identical to the algae's natural environment. The system relies on sunlight, and is typically less expensive to build and operate relative to the enclosed photobioreactor system. However, the open-pond system suffers from water loss due to evaporation. The system also suffers from potential contamination for unwanted algae species, and the lack of consistent optimal culture conditions (e.g., changes in pH, temperature and light penetration) for the algae.

The enclosed photobioreactor system ameliorates some of the deficiencies of the open pond system, such as evaporation and contamination. Enclosed photobioreactors cultivate algae in transparent materials (e.g., plastic or borosilicate glass tubes) by pumping nutrient-rich water through the system. Water circulation also ensures that the algae do not settle in the enclosure. The system also relies on sunlight. Since the system is enclosed, oxygen produced as a byproduct of photosynthesis needs to be removed, and carbon dioxide must be fed into the system to avoid carbon starvation. Additional disadvantages to the system include, expense to build, operate and maintain, scale-up light penetration, and formation of algal and bacterial biofilms.

Therefore, there continues to be a need for alternative composition and methods for improved, cost-effective and efficient biomass production systems, particularly large-scale systems. The present disclosure meets such needs by removing or minimizing the disadvantages of the open-pond system, while combining the advantages of the closed system, and further reduces costs associated with harvesting while increasing photosynthetic efficiency and biomass productivity.

### SUMMARY

The present disclosure describes algae based microphotobioreactor (μPBR) systems comprising a biocompatible polymer (e.g., hydrogel) containing algae and inorganic carbon, and methods for making such μPBRs.

In one aspect, the disclosure provides for a composition comprising a biocompatible polymer bead having inorganic carbon and algae. In another aspect, the biocompatible polymer is a homopolymer or heteropolymer or combination thereof. In yet another aspect, the biocompatible polymer comprises a polysaccharide.

In another aspect, the biocompatible polymer is a hydrogel foam.

In another aspect, the biocompatible polymer comprises cross-linked monomers selected from the group consisting or organic monomers, inorganic monomers and combinations thereof. In another aspect, the biocompatible polymer comprises cross-linked monomers selected from the group consisting of alginate, agar, carrageenins, cellulose, combination of silicone and/or siloxanes with polyacrlymide and combinations thereof.

In another aspect, the biocompatible polymer having the inorganic carbon and algae is in the form of a membrane with an average thickness of from 2 mm to 10 mm (or 2, 3, 4, 5, 6, 7, 8, 9 or 10 mm). In a related aspect, the membrane is contact with an aqueous layer.

In another aspect, the biocompatible polymer having the inorganic carbon and algae is in the form of beads having an average diameter of from about 0.1 to about 10 mm (or 0.1, 0.2, 0.3, 0.4, 0.5, 0.6, 0.7, 0.8, 0.9, 1, 2, 3, 4, 5, 6, 7, 8, 9 or 10 mm), preferably 0.2 to 5 mm, more preferably 0.2 to 3 mm. In a related aspect, beads are suspended in an aqueous solution.

In another aspect, the monomers of the biocompatible polymer are cross-linked with a multivalent cation. In a related aspect, the multivalent cation is selected from the group consisting of a metal cation, an amine, an amino acid derivative, a water-miscible organic solvent and combinations thereof. In another aspect, the metal cation is selected from the group consisting of calcium, magnesium, iron, copper, zinc, mangenses, potassium, sodium, ammonia, biocompatible Lewis acid metals and combinations thereof. In another aspect, the monomers of the biocompatible polymer are cross-linked with an anion. In a related aspect, the anion is selected from the group consisting of phosphate, selenate, nitrate, chloride sulfate and combinations thereof.

In another aspect, the volume of inorganic carbon in the biocompatible polymer is up to 60%. In a related aspect, the volume of inorganic carbon in the biocompatible polymer is from about 5% to about 60% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60%). In another aspect, the inorganic carbon is selected from the group consisting of carbon dioxide, carbonic acid, bicarbonate anion, carbonate and a combination thereof. In another aspect, the inorganic carbon forms pockets in the biocompatible polymer having an average diameter of from about 0.5 nm to about 10 nm (or 0.5, 1, 1.5, 2, 2.5, 3, 3.5, 4, 4.5, 5, 5.5, 6, 6.5, 7, 7.5, 8, 8.5, 9 or 10 nm).

In another aspect, the algae are modified to have increased light utilization efficiency compared to wild-type algae of the same strain. In a related aspect, the algae have a photosynthetic rate that is higher than wild-type algae of the same strain at saturating light. In another aspect, the algae have at least about 10% greater biomass than wild-type algae of the same strain. In a related aspect, the algae have at least about 15% greater biomass than wild-type algae of the same strain. In a related aspect, the algae have at least about 20% greater biomass than wild-type algae of the same strain. In a related aspect, the algae have at least about 25% greater biomass than wild-type algae of the same strain. In a related aspect, the algae have at least about 30% greater biomass than wild-type algae of the same strain.

In another aspect, the peripheral light harvesting antenna size of photosystem II of the algae is smaller than the peripheral light harvesting antenna size of photosystem II of wild-type algae of the same strain.

In another aspect, the ratio of chlorophyll a to chlorophyll b of green algae (Chlorophyta) is greater than the ratio of chlorophyll a to chlorophyll b of wild-type algae of the same strain. In a related aspect, the ratio of chlorophyll a to chlorophyll b of the algae is from about 3 to about 7 (or 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9 or 7). In another aspect, the chlorophyll b content of the algae is reduced by an RNAi mechanism.

In another aspect, the algae comprise a siRNA that targets the chlorophyllide a oxygenase (CAO) gene. In another aspect, the aglae's endogenous CAO gene levels are reduced compared to the CAO gene levels of a wild-type algae of the same strain. In another aspect, the translation activity of the CAO gene is reduced or inhibited with a nucleic acid binding protein 1 (NAB1). In another aspect, the algae is a transgenic algae expressing a protein comprising the amino acid sequence selected from the group consisting of SEQ ID NOs: 1, 2, 3 and combination thereof.

In a related aspect, the strain of algae is selected from the group consisting of Chlamydomonas reinhardtii, Chlorella sp., Synechocystis sp., Synechococcus, Anabaena sp., Cyclotella, Phaeodactylum sp., Crypthicodineum sp., Schizochytridum sp., Haematococcus sp., Arthrospira (Spirulina) sp., Dunaliella sp. and combination thereof.

In another aspect, the biocompatible polymer further comprises a light frequency-shifting agent. In a related aspect, the light frequency-shifting agent is red light emitting. In another aspect, the light frequency-shifting agent absorbs light comprising the light spectrum of from ultraviolet to green light and emits light comprising red light.

In another aspect, the light frequency-shifting agent is selected from the group consisting of a quantum dot, a fluorescent protein and a combination thereof. In a related aspect, the association between the light frequency-shifting agent and the biocompatible polymer is selected from the group consisting of a covalent bond, non-bonded interactions and a combination thereof.

In another aspect, the light frequency-shifting agent is a colloidal nanocrystal quantum dot. In another aspect, the colloidal nanocrystal quantum dot comprises an inner core having an average diameter of at least 1.5 nm and an outer shell, wherein the outer shell comprises multiple monolayers of an inorganic material. In another aspect, the colloidal nanocrystal quantum dot outer shell comprises at least four monolayers of inorganic material. In a related aspect, the colloidal nanocrystal quantum dot outer shell comprises from about four to twenty monolayers of inorganic material. In another aspect, the colloidal nanocrystal quantum dot exhibits an effective Stokes shift of at about least 75 nm. In another aspect, the colloidal nanocrystal quantum dot inner core comprises material selected from the group consisting of CuInS₂, Zn3P₂, GaP, GaAs, GaSb, InP, InAs, InSb, ZnS, ZnSe, ZnTe, CdSe, CdS, CdTe, PbS, PbSe, PbTe, and combinations thereof. In a related aspect, the colloidal nanocrystal quantum dot outer shell comprises material selected from the group consisting of ZnS, ZnSe, ZnTe, CdS, CdSe, CuGaS₂, GaP, Cu₂0, AlP, AlAs, GaS, SnS₂ and combinations thereof. In yet another aspect, the colloidal nanocrystal quantum dot inner core and outer shell comprise, respectively, CuInS₂ and ZnS, or CuInS₂ and ZnSe, or InP and ZnS, or InP and ZnSe, or Zn₃P₂ and ZnS.

In another aspect, the light frequency-shifting agent is a fluorescent protein. In another aspect, the fluorescent protein absorbs light comprising blue light and emits light comprising red light. In another aspect, the fluorescent protein is a fusion protein of a green fluorescent protein (GFP) and a red fluorescent protein (RFP), wherein the fusion protein absorbs light comprising blue light and emits light comprising red light.

In another aspect, the biocompatible polymer further comprises an exogenous agent that is capable of converting carbon dioxide to bicarbonate. In a related aspect, the association between the exogenous agent and the biocompatible polymer is selected from the group consisting of a covalent bond, non-bonded interactions and a combination thereof.

In a related aspect, the exogenous agent is a carbonic anhydrase enzyme. In a related aspect, the amino acid sequence of the carbonic anhydrase enzyme is selected from the group consisting of SEQ ID NOs: 1, 2, 3 and a combination thereof.

In another aspect, the disclosure provides for a method for preparing a biocompatible polymer having inorganic carbon and algae comprising the steps of: preparing a mixture by combining an aqueous biocompatible monomer solution with inorganic carbon; and combining the mixture with an aqueous solution having multivalent metal cations, thus forming the biocompatible polymer; and combining the biocompatible polymer with algae. In another aspect, the biocompatible polymer is a homopolymer or heteropolymer or combination thereof. In yet another aspect, the biocompatible polymer comprises a polysaccharide.

In another aspect, the biocompatible polymer is a hydrogel foam.

In another aspect, the biocompatible polymer comprises cross-linked monomers selected from the group consisting or organic monomers, inorganic monomers and combinations thereof. In another aspect, the biocompatible polymer comprises cross-linked monomers selected from the group consisting of alginate, agar, carrageenins, cellulose, combination of silicone and/or siloxanes with polyacrlymide and combinations thereof.

In another aspect, the biocompatible polymer having the inorganic carbon and algae is in the form of a membrane with an average thickness of from 2 mm to 10 mm (or 2, 3, 4, 5, 6, 7, 8, 9 or 10 mm). In a related aspect, the membrane is contact with an aqueous layer.

In another aspect, the biocompatible polymer having the inorganic carbon and algae is in the form of beads having an average diameter of from about 0.1 to about 10 mm (or 0.1, 0.2, 0.3, 0.4, 0.5, 0.6, 0.7, 0.8, 0.9, 1, 2, 3, 4, 5, 6, 7, 8, 9 or 10 mm), preferably 0.2 to 5 mm, more preferably 0.2 to 3 mm. In a related aspect, beads are suspended in an aqueous solution.

In another aspect, the monomers of the biocompatible polymer are cross-linked with a multivalent cation. In a related aspect, the multivalent cation is selected from the group consisting of a metal cation, an amine, an amino acid derivative, a water-miscible organic solvent and combinations thereof. In another aspect, the metal cation is selected from the group consisting of calcium, magnesium, iron, copper, zinc, mangenses, potassium, sodium, ammonia, biocompatible Lewis acid metals and combinations thereof. In another aspect, the monomers of the biocompatible polymer are cross-linked with an anion. In a related aspect, the anion is selected from the group consisting of phosphate, selenate, nitrate, chloride sulfate and combinations thereof.

In another aspect, the volume of inorganic carbon in the biocompatible polymer is up to 60%. In a related aspect, the volume of inorganic carbon in the biocompatible polymer is from about 5% to about 60% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60%). In another aspect, the inorganic carbon is selected from the group consisting of carbon dioxide, carbonic acid, bicarbonate anion, carbonate and a combination thereof. In another aspect, the inorganic carbon forms pockets in the biocompatible polymer having an average diameter of from about 0.5 nm to about 10 nm (or 0.5, 1, 1.5, 2, 2.5, 3, 3.5, 4, 4.5, 5, 5.5, 6, 6.5, 7, 7.5, 8, 8.5, 9 or 10 nm).

In another aspect, the algae are modified to have increased light utilization efficiency compared to wild-type algae of the same strain. In a related aspect, the algae have a photosynthetic rate that is higher than wild-type algae of the same strain at saturating light. In another aspect, the algae have at least about 10% greater biomass than wild-type algae of the same strain. In a related aspect, the algae have at least about 15% greater biomass than wild-type algae of the same strain. In a related aspect, the algae have at least about 20% greater biomass than wild-type algae of the same strain. In a related aspect, the algae have at least about 25% greater biomass than wild-type algae of the same strain. In a related aspect, the algae have at least about 30% greater biomass than wild-type algae of the same strain.

In another aspect, the peripheral light harvesting antenna size of photosystem II of the algae is smaller than the peripheral light harvesting antenna size of photosystem II of wild-type algae of the same strain.

In another aspect, the ratio of chlorophyll a to chlorophyll b of green algae (Chlorophyta) is greater than the ratio of chlorophyll a to chlorophyll b of wild-type algae of the same strain. In a related aspect, the ratio of chlorophyll a to chlorophyll b of the algae is from about 3 to about 7 (or 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9 or 7). In another aspect, the chlorophyll b content of the algae is reduced by an RNAi mechanism.

In another aspect, the algae comprise an siRNA that targets the chlorophyllide a oxygenase (CAO) gene. In another aspect, the aglae's endogenous CAO gene levels are reduced compared to the CAO gene levels of a wild-type algae of the same strain. In another aspect, the translation activity of the CAO gene is reduced or inhibited with a nucleic acid binding protein 1 (NAB1). In another aspect, the algae is a transgenic algae expressing a protein comprising the amino acid sequence selected from the group consisting of SEQ ID NOs: 1, 2, 3 and combination thereof.

In a related aspect, the strain of algae is selected from the group consisting of Chlamydomonas reinhardtii, Chlorella sp., Synechocystis sp., Synechococcus, Anabaena sp., Cyclotella, Phaeodactylum sp., Crypthicodineum sp., Schizochytridum sp., Haematococcus sp., Arthrospira (Spirulina) sp., Dunaliella sp. and combination thereof.

In another aspect, the biocompatible polymer further comprises a light frequency-shifting agent. In a related aspect, the light frequency-shifting agent is red light emitting. In another aspect, the light frequency-shifting agent absorbs light comprising the light spectrum of from ultraviolet to green light and emits light comprising red light.

In another aspect, the light frequency-shifting agent is selected from the group consisting of a quantum dot, a fluorescent protein and a combination thereof. In a related aspect, the association between the light frequency-shifting agent and the biocompatible polymer is selected from the group consisting of a covalent bond, non-bonded interactions and a combination thereof.

In another aspect, the light frequency-shifting agent is a colloidal nanocrystal quantum dot. In another aspect, the colloidal nanocrystal quantum dot comprises an inner core having an average diameter of at least 1.5 nm and an outer shell, wherein the outer shell comprises multiple monolayers of an inorganic material. In another aspect, the colloidal nanocrystal quantum dot outer shell comprises at least four monolayers of inorganic material. In a related aspect, the colloidal nanocrystal quantum dot outer shell comprises from about four to twenty monolayers of inorganic material. In another aspect, the colloidal nanocrystal quantum dot exhibits an effective Stokes shift of at about least 75 nm. In another aspect, the colloidal nanocrystal quantum dot inner core comprises material selected from the group consisting of CuInS₂, Zn3P₂, GaP, GaAs, GaSb, InP, InAs, InSb, ZnS, ZnSe, ZnTe, CdSe, CdS, CdTe, PbS, PbSe, PbTe, and combinations thereof. In a related aspect, the colloidal nanocrystal quantum dot outer shell comprises material selected from the group consisting of ZnS, ZnSe, ZnTe, CdS, CdSe, CuGaS₂, GaP, Cu₂0, AlP, AlAs, GaS, SnS₂ and combinations thereof. In yet another aspect, the colloidal nanocrystal quantum dot inner core and outer shell comprise, respectively, CuInS₂ and ZnS, or CuInS₂ and ZnSe, or InP and ZnS, or InP and ZnSe, or Zn₃P₂ and ZnS.

In another aspect, the light frequency-shifting agent is a fluorescent protein. In another aspect, the fluorescent protein absorbs light comprising blue light and emits light comprising red light. In another aspect, the fluorescent protein is a fusion protein of a green fluorescent protein (GFP) and a red fluorescent protein (RFP), wherein the fusion protein absorbs light comprising blue light and emits light comprising red light.

In another aspect, the biocompatible polymer further comprises an exogenous agent that is capable of converting carbon dioxide to bicarbonate. In a related aspect, the association between the exogenous agent and the biocompatible polymer is selected from the group consisting of a covalent bond, non-bonded interactions and a combination thereof

In a related aspect, the exogenous agent is a carbonic anhydrase enzyme. In a related aspect, the amino acid sequence of the carbonic anhydrase enzyme is selected from the group consisting of SEQ ID NOs: 1, 2, 3 and a combination thereof.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic comparing the current open-pond system with related limitation, with that of the application of the microphotobioreactor (μPBR) to an improved open-pond system with related benefits. As shown, the current open-pond system uses larger amounts of water and carbon dioxide gas compared to the application of the μPBR to an improved open-pond system.

FIG. 2 shows algae growth over a one week time period in carbon dioxide and nutrient rich hydrogel beads.

FIG. 3 is a graph showing the difference in water evaporation rates (mL/m²) over a five hours between a water dish covered with hydrogel foam beads and a water dish with no cover. The evaporation rate for the water dish covered with hydrogel foam beads is less than the evaporation rate of the water dish with no cover indicating that less water may be used with the μPBR system because of the reduced evaporation rate.

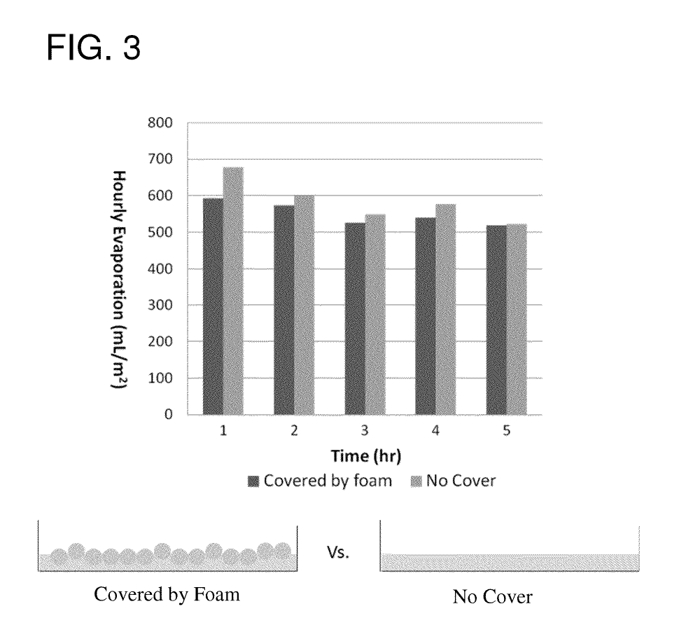

FIG. 4 shows the Stokes shift exhibit by the NQDs (light frequency-shifting agent) of the present disclosure (b) compared to traditional, smaller NQDs (a), as a function of signal intensity vs. wavelength.

FIG. 5 (a-h) shows the stability of NQDs having four (3a and 3b, control), seven (5c and 5d), twelve (3e and 3f) and nineteen (3g and 3h) monolayers. FIGS. 5b, 5d, 5f and 5h show data gathered from a single NQD, with intensity in arbitrary units (A.U.) on the y-axis vs. time in minutes on the x-axis. FIGS. 5a, 5c, 5e and 5g show data gathered from a plurality of NQDs, with intensity in arbitrary units on the y-axis vs. time in minutes on the x-axis.

FIG. 6 shows a comparison of the normalized Chl fluorescence yield of parental algae (CC-424), Chl b reduced transgenics (CR) and Chl b less mutant (cbs3). Chl fluorescence levels were measured under continuous, non-saturating illumination every 1 μs.

FIG. 7 (a-d) show the photosynthetic oxygen evolution and growth rates of Chl b reduced (CR), Chl b less (cbs3) and parental (CC-424 and CC-2677) strains. Light-dependent rates of photosynthesis for log-phase cultures grown photoautotrophically at 50 μmol photons m⁻² s⁻¹ measured in (a) the absence of NaHCO₃ or (b) presence of 10 mM NaHCO₃. (c) Photoautotrophic growth under limiting light intensities (50 μmol photons m⁻² s⁻¹). (d) Photoautotrophic growth under saturating light intensities (500 μmol photons m⁻² s⁻¹). Results represent the average and SE of three to four independent measurements.

FIG. 8 shows a schematic representation of the gene constructs used for NAB1 modulation of Chlorophyll b synthesis by in Chlamydomonas.

FIG. 9 shows changes in Chlorophyll a/b ratios in the complemented WT (CAO-4, 22), CC-2137 (also WT), N1BSCAO and altN1BSCAO transgenic clones during acclimation to low and high light.

FIG. 10 shows changes in Chlorophyll fluorescence induction in the complemented WT (CAO-4, 22), CC-2137 (also WT), N1BSCAO and altN1BSCAO transgenic clones during acclimation to low and high light.

### SEQUENCE LISTING

The nucleic and amino acid sequences listed in the accompanying Sequence Listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named Sequences.txt, created on Jan. 30, 2014, ˜60 KB, which is incorporated by reference herein. In the Sequence Listing:
SEQ ID NO: 1 is the amino acid sequence of wild-type human carbonic anhydrase II (CAII) protein.
SEQ ID NO: 2 is the amino acid sequence of a mutant form of the CAII protein (TS1) that exhibits increase thermal stability compared to the wild-type CAII. Compared to the wild-type sequence, Leu at position 100 of the sequence was changed to His; Leu at position 223 of the sequence was changed to Ser and Leu at position 239 was changed to Pro.
SEQ ID NO: 3 is the amino acid sequence of a mutant form of the CAII protein (TS3) that exhibits improved activity compared to the wild-type CAII; compared to the wild-type sequence, Leu at position 100 of the sequence was changed to His; Leu at position 223 of the sequence was changed to Ser and Leu at position 239 was changed to Pro Tyr at position 7 of the sequence was changed to Phe, and Asn at position 67 of the sequence was changed to Gln.
SEQ ID NO: 4 is the nucleic acid sequence of the CAO gene from Chlamydomonas reinhardtii.
SEQ ID NO: 5 is the nucleic acid sequence of the CAO gene from Volvox carteri f. nagariensis.
SEQ ID NO: 6 is the nucleic acid sequence of the CAO gene from Dunaliella salina.
SEQ ID NO: 7 is the nucleic acid sequence of the CAO gene from Nephroselmis pyriformis.
SEQ ID NO: 8 is the nucleic acid sequence of the CAO gene from Mesostigma viride.
SEQ ID NO: 9 is the amino acid sequence of a representative NAB 1 protein from Chlamydomonas reinhardtii.
SEQ ID NO: 10 is the amino acid sequence of a representative NAB 1 protein from Chlamydomonas incerta.
SEQ ID NO: 11 is the amino acid sequence of a representative NAB 1 protein from Volvox carteri f nagariensis
SEQ ID NO: 12 is the amino acid sequence of a representative NAB 1 protein from Physcomitrella patens subsp. Patens.
SEQ ID NO: 13 is the amino acid sequence of a representative NAB 1 protein from Zea mays.
SEQ ID NO: 14 is the amino acid sequence of a representative NAB 1 protein from Oryza sativa Japonica Group.
SEQ ID NO: 15 is the amino acid sequence of a representative NAB 1 protein from Chlorella variabilis.
SEQ ID NO: 16 is the amino acid sequence of a representative NAB 1 protein from Selaginella moellendorffi.
SEQ ID NO: 17 is the amino acid sequence of a representative NAB 1 protein from Vitis vinifera.
SEQ ID NO: 18 is the amino acid sequence of a representative NAB 1 protein from Triticum aestivum.
SEQ ID NO: 19 is the amino acid sequence of a representative NAB 1 protein from Cryptosporidium parvum Iowa II.
SEQ ID NO: 20 is the amino acid sequence of a representative NAB 1 protein from Arabidopsis thaliana.
SEQ ID NO: 21 is the amino acid sequence of an exemplary fluorescent protein, Katushka 9-5.
SEQ ID NO: 22 is the amino acid sequence of an exemplary fluorescent protein, Kat650-21.
SEQ ID NO: 23 is the amino acid sequence of an exemplary fluorescent protein, Kat670-23.
SEQ ID NO: 24 is the amino acid sequence of an exemplary fluorescent protein, KatX1.
SEQ ID NO: 25 is the amino acid sequence of an exemplary fluorescent protein, KatX2.
SEQ ID NO: 26 is the amino acid sequence of an exemplary fluorescent protein, Katusha9-5A.
SEQ ID NOs: 27 & 28 are forward and reverse primers used to amplify the first two exons and introns of the CAO gene.
SEQ ID NOs: 29 & 30 are the forward and reverse primers used to amplify the cDNA region spanning exons 1 and 2 of the CAO gene.
SEQ ID NOs: 31 & 52 are the forward and reverse primers used to confirm the presence of the CAO-RNAi and paramomycin resistance cassettes in the transgenics; SEQ ID NO: 31 binds within the PsaD promoter while SEQ ID NO: 52 binds within the CAO-RNAi cassette.
SEQ ID NOs: 32 & 33 are the forward primers binding within the Hsp70/Rbcs2 fusion promoter.
SEQ ID NOs: 34 & 35 are the forward and reverse primers used for amplification of the CBLP gene.
SEQ ID NOs: 36 & 37 are the forward and reverse primers used for the amplification of the CAO gene.
SEQ ID NOs: 38 & 39 (respectively, N1BSCAO-F and CAO-Rev) are the forward and reverse primers used to amplify the CAO gene for construction of NAB 1.
SEQ ID NO: 40 is a 13-bp NAB 1 binding site (NI BS) used in constructing a NAB1 regulated CAO gene construct.
SEQ ID NOs: 41 & 42 are forward and reverse primers CAOEx12GS_F and CAOEx12GS_R used in constructing a NAB1 regulated CAO gene construct.
SEQ ID NOs: 43 & 44 are forward and reverse primers CAOEx12CAS_F and CAOEx12CAS-R used in constructing a NAB1 regulated CAO gene construct.
SEQ ID NOs: 45 & 46 are forward and reverse primers PSLI18-F-seq and PSLi1-R-seq used in constructing a NAB1 regulated CAO gene construct.
SEQ ID NOs: 47 & 48 are two forward primers (CAO-F and altN1BSCAO-F, respectively) used in combination with reverse primer above to generate control plasmids in which the CAO gene was not preceded by the NAB 1 binding site (PSL18-CAO), or had an altered NAB 1 binding site (PSL18-altN1BS-CAO).
SEQ ID NOs: 49 & 50 are sequence primers PSL18-psaD-F and CAO-seq primers used to sequence plasmids PSL18-CAO, PSL18-N1 BS-CAO and PSL18-altN 1BS-CAO.
SEQ ID NO 51 is a mutagenized NAB 1 binding site that is different from LHCBM6 mRNA CDSCS by 4 bp.

### DETAILED DESCRIPTION

### I. Abbreviations

dsRNA double-stranded RNA

FCS fluorescence correlation spectroscopy

FRET fluorescence resonance energy transfer

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Bead: A bead, as used herein, refers to a spherical or semispherical biocompatible material having a diameter of from 0.1-10 mm (or 0.1, 0.2, 0.3, 0.4, 0.5, 0.6, 0.7, 0.8, 0.9, 1, 2, 3, 4, 5, 6, 7, 8, 9 or 10 mm) that is permeable or semi-permeable and used to encapsulate various components of the μPBR system. The preferred diameter of the bead is about 0.2 to 2 mm.

Biocompatible: The term biocompatible, as used herein, refers to synthetic and/or natural material that does not have a substantial negative impact on organisms, tissues, cells, biological systems or pathways and/or protein function relevant to the μPBRs disclosed herein.

Biomass: Biomass, as used herein, refers to any algal-based organic matter that may be used for carbon storage and/or as a source of energy (e.g., biofuels).

Blue Light: Blue light, as used herein, refers to visible light having a wavelength of from about 450 nm to about 495 nm. While a range is provided, it should be appreciated by one of ordinary skill in the art that light identified as having a specific color may have a wavelength that falls outside the range provided, and therefore such wavelength(s) that fall outside this range will also be included within the definition provided.

Carbon capture: Carbon capture, as used herein, refers to the sequestration of inorganic carbon (e.g., carbon dioxide) by algae.

Carbon fixation: Carbon fixation, as used herein, refers to the reduction of inorganic carbon (e.g., carbon dioxide) to organic compounds by algae.

Carbonic Anhydrase: Carbonic anhydrases (CAs), as used herein, are a family of enzymes that catalyze the reversible hydration/dehydration of carbon dioxide/bicarbonates. The enzymes are abundant in mammalian, plant, algae and bacteria. There are three distinct classes of CA enzymes—alpha (mammalian), beta (plant) and gamma (bacteria). While members of the different classes share very little sequence or structural similarity, they all perform the same function described above. The members of the alpha class of CA enzymes are encoded by the following genes: CA1, CA2, CA3, CA4, CA5A, CA5B, CA6, CA7, CA9, CA12, CA13, CA14 and CA15. The nucleotide sequence of the carbonic anhydrase genes, and the amino acid sequences of the protein for which these genes encode from the alpha, beta and gamma classes, are readily available in multiple database on-line. By way of example, the amino acid sequence of the wild-type human carbonic anhydrase II (CAII) protein is as follows:

(SEQ ID NO: 1)
MSHHWGYGKHNGPEHWHKDFPIAKGERQSPVDIDTHTAKYDPSLKPLSVS
YDQATSLRILNNGHAFNVEFDDSQDKAVLKGGPLDGTYRLIQFHFHWGSL
DGQGSEHTVDKKKYAAELHLVHWNTKYGDFGKAVQQPDGLAVLGIFLKVG
SAKPGLQKVVDVLDSIKTKGKSADFTNFDPRGLLPESLDYWTYPGSLTTP
PLLECVTWIVLKEPISVSSEQVLKFRKLNFNGEGEPEELMVDNWRPAQPL
KNRQIKASFK

Further, by way of example, the amino acid sequence of a mutant form of the CAII protein (TS1) that exhibits increase thermal stability compared to the wild-type CAII, and may be used within the context of the μPBRs disclosed in this patent application, is as follows (compared to the wild-type sequence, Leu at position 100 of the sequence was changed to His; Leu at position 223 of the sequence was changed to Ser and Leu at position 239 was changed to Pro):

(SEQ ID NO: 2)
MSHHWGYGKHNGPEHWHKDFPIAKGERQSPVDIDTHTAKYDPSLKPLSVS
YDQATSLRILNNGHAFNVEFDDSQDKAVLKGGPLDGTYRLIQFHFHWGSH
DGQGSEHTVDKKKYAAELHLVHWNTKYGDFGKAVQQPDGLAVLGIFLKVG
SAKPGLQKVVDVLDSIKTKGKSADFTNFDPRGLLPESLDYWTYPGSLTTP
PLLECVTWIVLKEPISVSSEQVSKFRKLNFNGEGEPEEPMVDNWRPAQPL
KNRQIKASFK

Further, by way of example, the amino acid sequence of a mutant form of the CAII protein (TS3) that exhibits improved activity compared to the wild-type CAII, and may be used within the context of the μPBRs disclosed in this patent application, is as follows (compared to the wild-type sequence, Leu at position 100 of the sequence was changed to His; Leu at position 223 of the sequence was changed to Ser and Leu at position 239 was changed to Pro Tyr at position 7 of the sequence was changed to Phe, and Asn at position 67 of the sequence was changed to Gln):

(SEQ ID NO: 3)
MSHHWGFGKHNGPEHWHKDFPIAKGERQSPVDIDTHTAKYDPSLKPLSVS
YDQATSLRILNNGHAFQVEFDDSQDKAVLKGGPLDGTYRLIQFHFHWGSH
DGQGSEHTVDKKKYAAELHLVHWNTKYGDFGKAVQQPDGLAVLGIFLKVG
SAKPGLQKVVDVLDSIKTKGKSADFTNFDPRGLLPESLDYWTYPGSLTTP
PLLECVTWIVLKEPISVSSEQVSKFRKLNFNGEGEPEEPMVDNWRPAQPL
KNRQIKASFK

The CA enzyme to be used to catalyze the reversible hydration/dehydration of carbon dioxide/bicarbonates within the μPBRs described herein may have an amino acid sequence having at least 30% preferably at least 40, 50, 60, 70, 75, 80, 85, 90, 95, 98, or 99% identity with CA enzymes listed above.

Chlorophyll: Chlorophyll is a green pigment found in the chloroplasts of algae and plants. It plays a critical in the photosynthetic process by absorbing light and transferring light energy by resonance energy transfer to the reaction centers of the photosystems. Chlorophyll a (Chl a) is a specific form of chlorophyll that absorbs light energy from the violet-blue and orange-red portions of the electromagnetic spectrum. Chlorophyll b (Chl b) is another specific form of chlorophyll that absorbs light energy primarily in the blue portion of the electromagnetic spectrum.

Chlorophyllide A Oxygenase (CAO): The CAO gene is encodes the CAO protein, which is responsible for the synthesis of Chlorophyll b by the oxidation of Chlorophyll a. The CAO gene from any strain of algae may be used within the context of the μPBRs disclosed in this patent application. By way of example, the nucleic acid sequence of the CAO gene from Chlamydomonas reinhardtii is as follows:

(SEQ ID NO: 4)
AGTTGTAGGGCCCTTGCATTAACGAAGGTTAGGCATCAGGCGGAGGCGCC
TGAACTATTTCAACGACTGAAGACCGGTCGCTCATTCCTTGCGCATTGCT
GCTTTGGTAGATGCGTGTTACCGCATAGAGCAGCCTGCTTGCAATTCAGT
TTTTGATCTCTAAGATAGAGCAGCGCCTGCAAAAGGCGCAGACGCTTTCG
TCAGATGCTTCCTGCGTCGCTTCAACGCAAGGCCGCTGCCGTTGGCGGTC
GCGGCCCCACCAACCAGAGTCGCGTGGCAGTTCGCGTCTCTGCTCAGCCG
AAGGAAGCTCCTCCCGCCTCGACACCCATCGTTGAGGACCCGGAGAGCAA
GTTCCGCCGCTATGGCAAGCATTTCGGCGGCATTCACAAGCTGAGCATGG
ATTGGCTTGATAGCGTTCCTCGCGTGCGCGTGCGCACCAAGGACTCTCGC
CAGCTGGACGATATGTTGGAGCTGGCAGTGCTCAACGAGCGCCTTGCGGG
TCGCTTGGAGCCCTGGCAGGCTCGTCAGAAGCTTGAGTACCTCCGTAAGC
GGCGGAAGAACTGGGAGCGCATTTTCGAGTACGTGACGCGTCAGGATGCG
GCCGCGACCCTGGCCATGATCGAGGAGGCAAATCGCAAGGTGGAGGAGTC
GCTGAGCGAGGAGGCACGCGAGAAGACTGCTGTAGGCGACCTCCGAGACC
AGCTGGAGTCGCTGCGCGCGCAGGTGGCGCAGGCGCAGGAGCGCCTTGCT
ATGACGCAGTCGCGCGTGGAGCAGAACCTACAGCGCGTGAATGAGCTGAA
GGCGGAGGCGACCACGCTAGAGCGCATGCGCAAGGCCTCGGACCTGGACA
TCAAGGAGCGCGAGCGCATCGCCATCTCCACTGTCGCCGCCAAGGGACCG
GCCTCGAGCAGCAGCAGCGCCGCCGCCGTCAGCGCCCCCGCCACGTCGGC
CACGCTGACGGTGGAGCGCCCCGCCGCCACCACGGTGACGCAGGAGGTGC
CGTCCACCAGCTACGGCACCCCCGTGGACCGCGCGCCGCGCCGCAGCAAG
GCGGCCATCCGGCGCAGCCGCGGGCTGGAAAGCAGCATGGAGATTGAGGA
GGGCCTGCGCAACTTCTGGTACCCCGCTGAGTTCTCAGCGCGCTTGCCGA
AGGACACGCTGGTGCCCTTTGAGCTGTTTGGCGAGCCGTGGGTGATGTTC
CGTGATGAGAAGGGGCAGCCCTCCTGCATCCGCGACGAGTGCGCACACCG
CGGCTGCCCGCTCAGCCTGGGCAAGGTGGTGGAGGGACAGGTCATGTGCC
CCTACCACGGCTGGGAGTTCAACGGCGACGGCGCCTGCACCAAGATGCCC
TCCACGCCCTTCTGCCGCAATGTGGGCGTTGCCGCGCTGCCTTGCGCGGA
GAAGGATGGCTTCATCTGGGTCTGGCCCGGCGACGGCCTGCCAGCGGAGA
CGCTGCCGGACTTCGCCCAGCCGCCAGAGGGCTTTCTGATCCACGCGGAG
ATCATGGTGGATGTGCCTGTGGAGCACGGCCTGCTGATTGAGAACCTGCT
GGACCTGGCGCACGCGCCGTTCACGCACACCAGCACCTTCGCGCGCGGCT
GGCCTGTGCCCGACTTCGTCAAGTTCCATGCCAACAAGGCGCTCTCGGGC
TTCTGGGACCCCTACCCCATCGACATGGCCTTCCAGCCGCCCTGCATGAC
GCTGTCCACCATCGGCCTGGCGCAACCCGGCAAGATTATGCGCGGCGTGA
CCGCCAGCCAGTGCAAGAACCACCTGCACCAGCTGCACGTGTGCATGCCC
TCCAAGAAGGGCCACACGCGGCTGCTGTACCGCATGAGCCTGGACTTCCT
GCCCTGGATGCGCCACGTGCCCTTCATCGACCGCATCTGGAAGCAGGTGG
CGGCGCAGGTGCTGGGCGAGGACCTGGTGCTGGTGCTGGGCCAGCAGGAC
CGCATGCTGCGCGGCGGCAGCAACTGGTCCAACCCCGCGCCCTACGACAA
GCTGGCGGTGCGCTACCGCCGCTGGCGCAACGGCGTAAACGCCGAGGTCG
CACGCGTGCGCGCCGGCGAGCCACCGTCCAACCCCGTGGCAATGAGCGCG
GGCGAGATGTTCTCGGTGGACGAGGATGACATGGACAACTAGAAGCCACG
TGGCGTGGATTGGCGAGCGGAGGTGGCAGGAGCGAGCATGGGCGTGGTGG
AGGATAGAGCGGCGAGGGCAGCTAGGGCCGTGGTGCAGGCGGCGGGGTGT
ACATGGCTGAGGTGGGCAGCGGCAGGCGCAGCAAACGCGGCTAGAGACCG
AGGCCAATTCATGCAGGAGCCCGTCGAGAGCGTGTTAGGGTCAGCTTCAG
GGTATTACGGGTGCATGAGTGTGGTAGGTACAGGTGGTTAGGCGTCCATG
TTTGAGCCACTGCGTGTGCAAATAGTGCTTGGACAGCCGTGCGCCAGGTG
CGTAATAGTATGTCCATGGATCACTGAACAATGAGAAGATACAATCTGTG
GACTCATACATAGTGCGGGGTTTGTTATCAGATGTCGGGCGGCCGCGCAG
TGTGTGTCGCTGGAAGGTATCGGCAATGTGCGAGGAAGTGTACACTGTTG
GTGCCTGTAGCTAGTGCGCTTGGTGCGTCGCGTGTGTGCAAGTCATGGTT
CCTGGCGGGAGTCAGCGTGCAATGGACCACTTCATCCGCTGCCCGGATGT
TAAGGTACGTGTGCGTTGAGGATGAGAGTCTGGTTGGAGAGCCAGTGGCA
GAGGGGCAAGGCCCTTTGCTACTTTGTGATCGCGTGCTCATCGTTGCTAT
TGTTTTTTGCCGGCGTAAGCGGCGTGGTGGAGGACGCAACGTGTGCTGCA
GCTGGGTGTTGAGATCGAGGGACCCGAAGCACACGGCTCAGAAGAACGTT
TTCATCCAGCCTGGAGAGGTGTGCGTGTGCTGCGGTCAATGAGTTTGCGC
TGGCGTCCAGAACGACTCTTGGGGATGCGTTGTTGAGACGTAGGGTTAGG
GTTTGGTATGAAGTGCACCGAAAGAGCAGCAGTGAGTGGCAAGTGCCCCT
TTCTGCGCTGTTCGGCCCCTGCAAGTTGAAGTAGTTCTTGGATGCAGTCC
CAACCCGGGCATGCGGTCGGTGCTGGTGTATCAAACAATCTGGAGTTTTG
GTGTCCGGCCATGGGTGTCGCTGTGTGTGTTCATTTCGGGGAGGCTGAGT
TCCAACGGCCCCTAGGCCGCCGCTTGGGGGTCTCCGCTGTGTACCATTGA
ATCGGTCTGCAGACTGGGTTCCGTACCCAATTAATTTTGTTTCGCGGTCT
TTCATAACGCGTAAGAACCCGCGTCGGAAGAGTGGAAATGGTTGGTGGTG
AGAAGGAGCGGCTCGTCAGTACGGAGGTGTTGACGGAGCTCCAGTGAGAA
AGTACAGCGAAATACTGTAACGCTAGCTGCTGAAAAAAAAAAAAAAAAA

Representative species and GenBank accession numbers for various species of chlorophyll A oxygenase are listed below, and genes from other species may be readily identified by standard homology searching of publicly available databases.

By way of example, the nucleic acid sequence of the CAO gene from Volvox carteri f. nagariensis is as follows:

(SEQ ID NO: 5)
ATGCTTCCAGCACAAAGACAGTGCAGGACGTCCGCCTGCCAAGGCAGGGG
CATTATAAGCAAGAGGACTATCCGTGCTGACTTTAAAGTCCATGCGTCAG
TATCACAGCAGCCTTCTTCAGACAAGCCTGAGCAACAGGCTGTACCGTCT
ATCGTCGAGGACCCTGAAGCGAAGTTTCGGCGTTATGGCAAGCATTTCGG
TGGTATCCATAAGCTAAATCTGGATTGGCTGGAGGCAGTTCCGCGTGTGC
GTGTTCGGACCAAAGATTCACGGCAGCTCGACGAGCTGTTGGAGCTGGCA
GTGCTCAATGAGCGCCTTGCGGGACGCTTGGAGCCTTGGCAGGCACGCCA
GAAGCTTGAGTATCTGCGTAAGCGCCGGAAGAACTGGGAGCGCATCTTTG
AGTACGTCACTAAGCAGGACGCTGCTGCCACGCTAGCCATGATCGAGGAG
GCCAACCGAAAGGTGGAGGAAGCCTTGTCGGAAGAGGCACGCGAGCGAAC
AGCAGTGGGAGATTTGCGGGAGCAGCTTCAAGTCCTGCAACGCCAGGTGC
AGGAGGCGCAGGAGCGGCTTCAGCTCACGCAAGCACGTGTGGAGCAGAAC
CTGAACCGCGTGAATGAGCTGAAGGCAGAGGCGGTCGGCCTGGAGCGGAT
GCGAAACGGAAGGATGGGTGGCGATCGCAAGAAGGAGCTCCAGGTGGCGG
CGCCAGTCGCTGTCACTGCCGCGGCGTCGGCGGCACGTCCTGCTGTTTCT
GCTACGGCAGTGGCGGAATCAGTCCCCGCGGCCATCGTGACAGTGGAGCC
CCCTACCAGGAGCTATACCCCCAATGGCTCGTCCGATGGCACGTCGGTTG
TCGCCCCACCAGGTCGTCGCAGCAAGGTAGCCATCCGACGGGGTCGCGGT
CTGGAGAGCAGCTTGGACTTCGAGCCAGGCCTTCGCAACTTTTGGTACCC
TGCGGAGTTTTCAGCGAAGCTGGGTCAGGACACGCTGGTTCCCTTCGAGC
TGTTTGGGGAGCCCTGGGTCCTGTTCCGCGACGAGAAGGGGCAGCCCGCT
TGCATCAAGGACGAATGCGCACATCGGGCCTGCCCGTTGTCGCTTGGAAA
GGTGGTAGAGGGGCAGGTTGTGTGCGCGTACCACGGCTGGGAGTTCAACG
GCGATGGCCACTGCACCAAGATGCCCTCCACGCCGCATTGCCGCAACGTG
GGGGTATCGGCGCTGCCCTGCGCTGAGAAGGATGGCTTCATCTGGGTGTG
GCCTGGAGACGGACTCCCGGCGCAGACGCTCCCCGACTTCGCACGCCCAC
CGGAGGGCTTTCAAGTGCACGCTGAGATTATGGTGGACGTGCCGGTGGAG
CATGGCCTGCTCATGGAGAACCTTTTGGATCTGGCGCATGCGCCATTCAC
CCACACCACAACTTTTGCGCGCGGCTGGCCCGTGCCTGACTTCGTCAAGT
TCCACACCAACAAATTACTATCGGGATACTGGGACCCCTACCCCATCGAC
ATGGCTTTCCAGCCGCCTTGCATGGTTCTGTCCACGATTGGCTTGGCGCA
ACCTGGCAAGATTATGCGCGGCGTGACGGCATCGCAATGCAAGAACCATC
TGCACCAGCTCCATGTGTGCATGCCGTCGAAGAAGGGCCACACGCGGCTG
CTGTACCGCATGAGCCTAGACTTCCTGCCGTGGATGCGCTACGTGCCGTT
TATTGACAAGGTCTGGAAGAATGTTGCGGGCCAGGTGTTGGGCGAGGACC
TGGTGCTGGTGCTGGGGCAACAGGATCGTTTGCTGCGCGGCGGGAACACC
TGGTCGAACCCGGCGCCGTACGACAAGCTGGCGGTACGATACCGCCGCTG
GCGCAACTCGGTCAGTCCCGATGGCGCTGGCCTTGACGGCCCGGCGCCAC
TGAACCCAGTGGCGATGAGCGCCGGGGAGATGTTTTCAATTGATGAAGAT
GAGCAGGATCCGCGGATGCAGTGA

By way of example, the nucleic acid sequence of the CAO gene from Dunaliella salina is as follows:

(SEQ ID NO: 6)
TCAACAGGGGTTGGGGCCATGCAATCAAAGCTCTTGGGGCTTCAAGACGA
GATTAGTGAGGCAAGGGACAAGCTGCGTACCTCAGAGGCAAGGGTGGCAC
AAAACCTCAAGCGTGTGGATGAGTTGAAGGCTGAGGCGGCTTCCTTGGAG
CGCATGCGCCTGGCCAGCAGCTCAAGCACTGACAGCACAGTCAGCATTGC
CAGCAGGGGGGGCGCAGCTGTGGCTGCAACCACGAGCGTACCGGACCATG
TGGAGAGGGAAGGGATCCAGAGCAGGGTGCGGGGCAGTGGCATGGCCTCA
ACAAGCTACCCCTCCCATGTACCTCAGCCGAGCCAGGCAGTGAGACGGGG
CCCTAAACCGAAGGACAGCAGGCGACTGAGAAGCAGCCTGGAGCTGGAAG
ACGGCCTGCGCAACTTCTGGTACCCGACCGAGTTTGCGAAGAAGCTGGAG
CCGGGCATGATGGTGCCCTTTGACTTGTTCGGCGTGCCGTGGGTGCTGTT
CCGAGATGAGCACAGCGCCCCCACCTGCATCAAGGACTCCTGCGCGCACC
GCGCATGCCCGCTGTCACTGGGCAAGGTCATCAACGGCCACGTGCAGTGC
CCCTACCATGGCTGGGAGTTTGACGGGAGCGGCGCGTGCACCAAGATGCC
CAGCACGCGCATGTGCCATGGCGTGGGCGTGGCCGCGCTGCCGTGCGTGG
AGAAGGACGGCTTTGTGTGGGTGTGGCCTGGGGATGGGCCCCCACCTGAC
CTGCCGCCGGACTTCACAGCCCCCCCTGCAGGCTATGACGTGCACGCAGA
GATCATGGTGGATGTGCCTGTGGAGCACGGCCTGCTGATGGAGAACTTAC
TTGATCTGGCCCACGCGCCCTTCACCCACACCACCACCTTTGCGCGGGGC
TGGCCCATCCCAGAGGCTGTGCGCTTCCATGCCACCAAGATGCTGGCAGG
TGACTGGGACCCCTACCCCATCAGCATGTCTTTTAACCCCCCCTGCATTG
CGCTGTCAACCATCGGGCTGTCGCAGCCTGGCAAGATCATGCGCGGCTAC
AAGGCAGAGGAGTGCAAGCGCCACCTACACCAGCTGCACGTGTGCATGCC
CTCCAAGGAGGGCCACACGCGCCTGCTGTACCGCATGAGCCTTGACTTCT
GGGGCTGGGCTAAGCACGTGCCATTTGTGGATGTGCTGTGGAAGAAGATT
GCTGGCCAGGTGCTGGGTGAGGACCTGGTGCTGGTGCTGGGGCAGCAGGC
TCGCATGATTGGCGGCGACGACACCTGGTGCACGCCCATGCCGTACGACA
AGCTGGCTGTGCGGTACCGGAGGTGGCGGAACATGGTGGCTGATGGTGAG
TACGAGGAGGGGTCTCGGAATCGCTGCACAAGCCAATATGACAGCTGGCC
AGATGTTTGACTCCCACGATGATGAGGATCTGTATGAGCATCAGCGCCAT
GATGAGGGGAACCTGCAGGGCCAGCAAAGCAGCGTTTTTGCTGCAAGGAA
GTGAGGGCATTCATCCTAGGTTTTTGCTTGAGCAGAAGGAGAGGCTTATA
GGATGGTAGAATTGATTGTAAAATTTTGTAACATGCTTGGTGGTTCAATG
GTTCCTGTACTTGATGACTTGTAGAATTTTTCCCGTCGAGGGTGTTCACA
CTGTTAAGTGCTATGTTGGCGGTGACTGAGGATGCATAATTGCGCTGTCC
CACCATGCATACTGTTGCCAGTTTTAAACGGATTTCATGTTGTCTCTCCA
GTTTTGATGGATTGCTGGATGGTTTGTTTTGGTCTCCCCTTTAATTTCTT
TAATTTGCCCTACTAAATGGGCTCTCAGTAGAACATGTGGTTGGAAATCT
GTAAGGTTCAAGAACATTT

By way of example, the nucleic acid sequence of the CAO gene from Nephroselmis pyriformis is as follows:

(SEQ ID NO: 7)
TGCGGTGGAGTTCACTTCGCGCTTGGGGAAGGACATCATGGTTCCGTTTG
AGTGCTTCGAGGAGTCCTGGGTACTCTTCCGCGACGAGGACGGCAAGGCG
GGCTGCATCAAGGACGAGTGCGCGCACCGCGCTTGCCCGCTCTCGCTCGG
CACGGTGGAGAACGGCCAGGCGACGTGCGCGTACCACGGCTGGCAGTTCA
GCACTGGGGGGGAGTGCACCAAGATCCCGTCGGTCGGCGCGCGGGGCTGC
TCGGGCGTGGGCGTGCGCGCCATGCCCACCGTGGAGCAAGATGGCATGAT
CTGGATCTGGCCCGGGGACGAGAAGCCCGCCGAGCACATCCCGTCCAAGG
AGGTGCTGCCGCCCGCGGGCCACACCCTCCACGCGGAGATAGTGCTGGAC
GTGCCCGTGGAGCACGGCCTGCTGCTGGAGAACCTCCTGGACCTGGCGCA
CGCGCCCTTCACCCACACGTCCACGTTCGCCAAGGGCTGGGCGGTCCCGG
AACTCGTCAAGTTCTCCACGGACAAGGTGCGCGCGCTCGGGGGCGCGTGG
GAACCTTACCCCATCGACATGAGCTTCGAGCCGCCCTGCATGGTGCTGTC
CACCATCGGGCTCGCGCAGCCGGGCAAGGTAGACGCGGGCGTGCGCGCGT
CCGAGTGCGAGAAGCACCTGCACCAGCTGCACGTGTGCATGCCCTCGGGC
GCGGGGAAGACGCGCCTGCTGTACCGCATGCACCTCGACTTCATGCCGTT
CCTCAAATACGTGCCGGGCATGCACCTGGTGTGGGAGGCCATGGCCAACC
AGGTGCTGGGGGAGGACCTGAGGCTGGTGCTGGGGCAGCAGGACAGGCTG
CAGAGGGGCGGGGACGTGTGGAGCAACCCCATGGAGTACGACAA

By way of example, the nucleic acid sequence of the CAO gene from Mesostigma viride is as follows:

(SEQ ID NO: 8)
GACGAGGACGGCCGCGTGGCGTGCCTGCGGGATGAGTGCGCGCACCGTGC
ATGCCCCCTGTCACTGGGCACGGTGGAGAACGGGCACGCGACCTGCCCCT
ACCATGGCTGGCAGTACGACACGGACGGCAAGTGCACAAAGATGCCGCAG
ACGCGGCTGCGCGCGCAGGTGCGCGTGTCCACCCTGCCCGTGCGCGAGCA
CGACGGCATGATCTGGGTGTACCCAGGGTCCAACGAGCCGCCCGAGCACC
TGCCGTCGTTCCTGCCCCCCAGCAACTTCACGGTGCACGCCGAGTTGGTG
CTGGAGGTGCCCATCGAGCACGGGCTGATGATCGAGAACCTGCTGGACCT
GGCACACGCGCCCTTCACGCACACCGAGACCTTTGCCAAGGGATGGTCGG
TCCCGGACTCTGTCAACTTCAAGGTCGCCGCGCAGTCGCTGGCGGGGCAT
TGGGAGCCGTACCCCATCAGCATGAAGTTTGAGCCGCCGTGCATGACGAT
CTCGGAAATCGGGCTGGCCAAGCCCGGGCAGTTGGAGGCCGGCAAGTTCA
GTGGCGAGTGCAAGCAGCACCTGCACCAGCTGCACGTGTGCATGCCCGCG
GGGGAGGGCCGCACGCGCATCCTCTACCGCATGTGCCTCGACTTTGCGCA
CTGGGTCAAGTACATACCCGGAATCCAGAATGTGTGGTCGGGCATGGCGA
CGCAGGTGCTTGGGGAGGACCTGCGGCTGGTGGAGGGGCAGCAGGATCGC
ATGCTGCGCGGCGCGGACATCTGGTACAACCCGGTCGCCTATGACAAGCT
GGGCGTGCGGTACCGCAGCTGGCGCCGCGCGGTCGAGCGCAACACGCGCA
GCCGGTTCATCGGGGGCCAGGAGAAGCTTGCGCCCGAGGGTAGAGACTAG
TGAGCAAAAGGGGTGACTGCTCCACTGTACCTTCATGGCCGAGCAGCCAG
CTGGTACAGGCCTGACACCGTGGCAAGCCTGCACTTGGGCCATGCAGCGG
GTTAAGGTTGAGGCTTCTGATGGCAACCCTTGTCCGGTCTATTGTACAAA
ACGAGGAACGGAGAACATGGCTCCATTGCAACTGTGAGATGTTGAGGATG
CATGCTGCTACAAGGTGCCAGCAAGGTCTGTCACAGGGATGCTCCAGCAT
GACCAATGGGTGCCATTGCTTGAAATGGATATGTGCTAACAGGGGGGGAT
TTACTCTTTGCTGCCCCAGTGTANANATCATGGCCAGGATGATACATTCA
TCNCCAATCTGCAGGGTACNTGTGAAANAACCTGNTGGNNTTGCATGCCT
TATCCNTTCCNANTGANAANANTTTTGNTGAGGGGCNCTTNCNGCTTNTT
ACCNAAAAANNNCTTGCCNNAAAAAAAAA

The chlorophyll A oxygenase gene to be used as a target for modulation, suppression or deletion may have a nucleotide sequence having at least 30% preferably at least 40, 50, 60, 70, 75, 80, 85, 90, 95, 98, or 99% identity with chlorophyll A oxygenase listed above.

Cross-Link: The term cross-link, as used herein, refers to the processing of forming a bond (e.g., covalent or ionic) or link between two monomers, between two polymers or between a monomer and a polymer.

Fluorescence intermittency: Fluorescence intermittency, also known as “blinking,” as used herein means that a NQD exhibits one or more periods of time in which fluorescence emission ceases and/or is interrupted.

Fluorescent Protein: A fluorescent protein is a protein that emits fluorescence when exposed to certain forms of electromagnetic radiation (e.g., visible light and UV light). Non-limiting examples of fluorescent proteins includes green fluorescent protein (GFP) and its mutant forms and derivatives including blue, cyan and yellow fluorescent proteins, and red fluorescent protein (RFP) and its mutant forms and derivatives. Further included are proteins capable of absorbing light in one portion of the electromagnetic spectrum (e.g., blue and/or green light) and emitting light in another portion of the electromagnetic spectrum (e.g., red light). Specific examples include red fluorescent proteins, LSS-mKate1 and LSS-mKate 2 (see Piatkevich, K. D., et al., PNAS, 107 (12), 5369-5374 (2010)).

Foam: A foam, as used herein, is a material or substance that is formed by trapping pockets of gas in a liquid, solid or gel, which gas pockets may be polydisperse (pockets of different sizes) or monodisperese (pocket of uniform size) within the liquid or solid.

Green Light: Green light, as used herein, refers to visible light having a wavelength of from about 492 nm to about 577 nm. While a range is provided, it should be appreciated by one of ordinary skill in the art that light identified as having a specific color may have a wavelength that falls outside the range provided, and therefore such wavelength(s) that fall outside this range will also be included within the definition provided.

Hydrogel: A hydrogel, as used herein, refers to a polymer made of natural and/or synthetic material that absorbs and retains aqueous solutions.

Hydrophobic: A hydrophobic (or lipophilic) group is electrically neutral and nonpolar, and thus prefers other neutral and nonpolar solvents or molecular environments. Examples of hydrophobic molecules include alkanes, oils and fats.

Hydrophilic: A hydrophilic (or lipophobic) group is electrically polarized and capable of H-bonding, enabling it to dissolve more readily in water than in oil or other “non-polar” solvents.

Inorganic Carbon: Inorganic carbon, as used herein, includes carbon dioxide, carbonic acid, bicarbonate ion and carbonate.

Label: A detectable compound or composition that is conjugated directly or indirectly to another molecule (such as a nucleic acid molecule or protein, for instance an antibody) to facilitate detection of that molecule. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lewis Acid Metals: Lewis acid metals, as used herein, may include copper, silver, beryllium, magnesium, zinc, cadmium, boron, tin, iron, cobalt and nickel.

Ligand-independent: Ligand-independent, or other grammatical variations thereof, as used in the present invention means that the properties of the NQDs described herein are substantially unaffected by the presence or absence of ligands, or the identity of the ligands.

Light Frequency-Shifting Agent: A light frequency-shifting agent (LFSA), as used herein, refers to material that exhibits a different excitation and emission spectra. Light frequency-shifting agents may typically have a Stokes shift of at least about 10 nm, 20 nm, 30 nm, 50 nm, 75, nm, 100 nm or about 135 nm. An LFSA may also be referred to as a light-shifting agent (LSA). Non-limiting examples of LFSA's include quantum dots and fluorescent proteins.

Light-Utilization Efficiency: Light-utilization efficiency refers to the best use of light after it is absorbed by light harvesting complexes (LHC). Generally, light utilization efficiency is compromised at high light intensities or also at low light when more light is absorbed by LHCs and that results in loss of energy by energy dissipation to reduce photodamage.

Membrane: A membrane, as used herein, refers to film-like structure made of biocompatible material having a thickness of from 0.1-10 cm (or 0.1, 0.2, 0.3, 0.4, 0.5, 0.6, 0.7, 0.8, 0.9, 1, 2, 3, 4, 5, 6, 7, 8, 9 or 10 cm) that is permeable or semi-permeable and used to hold various components of the μPBR system.

Monolayer: A monolayer, as used herein, refers to a quantum dot, and means an amount of material deposited onto the core or onto previously deposited monolayers, that results from a single act of deposition of the shell material. The exact thickness of a monolayer is dependent upon the material. By way of example only, a monolayer may have a thickness of about 0.35 nm.

Non-bonded interactions: Non-bonded interaction is a chemical bond that does not involve the sharing of pairs of electrons between atoms. Examples of non-bonded interactions includes hydrogen bonds, ionic bonds (electrostatic bonds), van der Waals forces and hydrophobic interactions.

On-time fraction: On-time fraction, as used herein, means the fraction of total observation-time during continuous excitation that a single-NQD is exhibiting fluorescence emission, or is “on,” where “continuous excitation” means essentially uninterrupted excitation by a suitable excitation source, one non-limiting example of which is a 532 nm, 205 mW continuous wave laser.

Orange Light: Orange light, as used herein, refers to visible light having a wavelength of from about 590 nm to about 622 nm. While a range is provided, it should be appreciated by one of ordinary skill in the art that light identified as having a specific color may have a wavelength that falls outside the range provided, and therefore such wavelength(s) that fall outside this range will also be included within the definition provided.

Percent Identity: Percent identity, as used herein in the context of two or more nucleic acids or peptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection.

Peripheral Light Harvesting Antenna: The peripheral light harvesting antenna, as used herein, refers to the peripheral antenna or light harvesting complex (LHC) of the photosystem II.

Photobleaching: Photobleaching, as used herein, means that fluorescence of a light-shifting agent ceases, which results in irreversible darkening.

Photosynthetic Rate: The photosynthetic rate, as used herein, generally refers to the rate of conversion of inorganic carbon (e.g., carbon dioxide) to organic carbon with light as an energy source. This rate may be measured by measuring the uptake of inorganic carbon (e.g. carbon dioxide) by the algae, or the production of oxygen by the algae, the production of carbohydrates within the algae or by the dry mass of the algae. The methods used to measure photosynthetic rate are well-known in the art, and it would be obvious to those skilled in the art of how to determine the photosynthetic rate by measuring any one or more of the above.

Photosystem I: Photosystem I (PSI) refers to the integral membrane protein complex that uses light energy to mediate electron transfer from plastocyanin to ferredoxin. PSI system consist of several components, the main ones being the antenna complex and the P700 reaction center. The antenna complex is composed of molecules of chlorophyll and carotenoids mounted on two proteins. These pigment molecules transmit the resonance energy from photons when they become photoexcited. Antenna molecules can absorb all wavelengths of light within the visible spectrum. The number of these pigment molecules varies from organism to organism. For instance, the cyanobacterium Synechococcus elongatus (Thermosynechococcus elongatus) has about 100 chlorophylls and 20 carotenoids, whereas spinach chloroplasts have around 200 chlorophylls and 50 carotenoids. Located within the antenna complex of PS I are molecules of chlorophyll called P700 reaction centers. The energy passed around by antenna molecules is directed to the reaction center. There may be as many as 120 or as few as 25 chlorophyll molecules per P700. The P700 reaction center is composed of modified chlorophyll a that best absorbs light at a wavelength of 700 nm, with higher wavelengths causing bleaching. P700 receives energy from antenna molecules and uses the energy from each photon to raise an electron to a higher energy level. These electrons are moved in pairs in an oxidation/reduction process from P700 to electron acceptors. P700 has an electric potential of about −1.2 volts. The reaction center is made of two chlorophyll molecules and is therefore referred to as a dimer. The dimer is thought to be composed of one chlorophyll a molecule and one chlorophyll a′ molecule (p700, webber). However, if P700 forms a complex with other antenna molecules, it can no longer be a dimer.

Ferredoxin (Fd) is a soluble protein that facilitates reduction of NADP⁺ to NADPH. Fd moves to carry an electron either to a lone thylakoid or to an enzyme that reduces NADP⁺. Thylakoid membranes have one binding site for each function of Fd. The main function of Fd is to carry an electron from the iron-sulfur complex to the enzyme ferredoxin-NADP⁺ reductase.

Plastocyanin is a metallic protein containing a copper atom and with patches of negative charge. After an electron is carried to a cytochrome complex, it is passed on to plastocyanin. Plastocyanin binds to cytochrome though little is known about the mechanism of this binding. Plastocyanin then transfers the electron directly to the P700 reaction center in the PS I antenna complex.

Photosystem II: Photosystem II (PSII) refers to the first protein complex in the light-dependent reactions. The enzyme captures photons of light to energize electrons that are then transferred through a variety of coenzymes and cofactors to reduce plastoquinone to plastoquinol. The energized electrons are replaced by oxidizing water to form hydrogen ions and molecular oxygen. By obtaining these electrons from water, photosystem II provides the electrons for all of photosynthesis to occur. The hydrogen ions (protons) generated by the oxidation of water help to create a proton gradient that is used by ATP synthase to generate ATP. The energized electrons transferred to plastoquinone are ultimately used to reduce NADP⁺ to NADPH or are used in cyclic photophosphorylation. The core of PSII consists of a pseudo-symmetric heterodimer of two homologous proteins D1 and D2. Unlike the reaction centers of all other photo systems which have a special pair of closely spaced chlorophyll molecules, the pigment that undergoes the initial photoinduced charge separation in PSII is a chlorophyll monomer. Because the positive charge is not shared across two molecules, the ionised pigment is highly oxidizing and can take part in the splitting of water.

Polymer: A polymer, as used herein, refers to a synthetic or natural material made up of repeating structural units (monomers) through a process of cross-linking (polymerizing) the structural units (monomers) together. A homopolymer is a polymer made up of a single repeating structural unit. A copolymer is a polymer made up of a mixture of two or more repeating structural units that differ structurally from one another.

Quantum Dot: A quantum dot, as used herein, refers to material having semiconductor properties and the ability to emit photons upon absorption of energy (e.g., light and/or electricity). As used herein, the terms “colloidal nanocrystal quantum dot”, “nanocrystal quantum dot” (or NQD), may be used interchangeably with the terms “quantum dot”, “nanocrystal”, “semiconductor quantum dot”, and other similar terms that would be familiar to one of skill in the art.

Red Light: Red light, as used herein, refers to visible light having a wavelength of from about 620 nm to about 780 nm. While a range is provided, it should be appreciated by one of ordinary skill in the art that light identified as having a specific color may have a wavelength that falls outside the range provided, and therefore such wavelength(s) that fall outside this range will also be included within the definition provided.

RNAi: RNA interference (RNAi), as used herein, refers to the cellular process of sequence specific, post-transcriptional gene silencing mediated by small inhibitory nucleic acid molecules, such as a dsRNA that is homologous to a portion of a targeted messenger RNA or other expressed RNA (e.g., siRNA or miRNA).

Stokes-Shift: Stokes shift, as used herein, refers to the difference in wavelength between the maxima of the absorption and emission spectra of a material.

Ultraviolet Light: Ultraviolet light, as used herein, refers to light having a wavelength of from about 10 nm to 400 nm. While a range is provided, it should be appreciated by one of ordinary skill in the art that light identified as having a specific color may have a wavelength that falls outside the range provided, and therefore such wavelength(s) that fall outside this range will also be included within the definition provided.

Violet Light: Violet light, as used herein, refers to visible light having a wavelength of from about 380 nm to 455 nm. While a range is provided, it should be appreciated by one of ordinary skill in the art that light identified as having a specific color may have a wavelength that falls outside the range provided, and therefore such wavelength(s) that fall outside this range will also be included within the definition provided.

Wild-type: Wild-type, as used herein, refers to the phenotype of the typical form of a species as it occurs in nature.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean±20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g., “or”) should be understood to mean either one, both, or any combination thereof of the alternatives

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview

Further provided is a composition comprising a biocompatible polymer having at least 10% by volume inorganic carbon and algae. In a related aspect, the volume of inorganic carbon in the biocompatible polymer is from about 5% to about 60% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60%). In another aspect, the inorganic carbon is selected from the group consisting of carbon dioxide, carbonic acid, bicarbonate anion, carbonate and a combination thereof. In another aspect, the inorganic carbon forms pockets in the biocompatible polymer having an average diameter of from about 0.5 nm to about 10 nm (or 0.5, 1, 1.5, 2, 2.5, 3, 3.5, 4, 4.5, 5, 5.5, 6, 6.5, 7, 7.5, 8, 8.5, 9 or 10 nm).

In another aspect this disclosure provides a composition comprising a biocompatible polymer having inorganic carbon and algae, wherein the algae is modified to have increased light utilization efficiency compared to wild-type algae of the same strain.

In another aspect, the disclosure provides algae having a photosynthetic rate of at least 2-fold higher than wild-type algae of the same strain at saturating light.

In another aspect, the biocompatible polymer further comprises activated charcoal.

In another aspect, the biocompatible polymer having the inorganic carbon and algae is in the form of a membrane. In a related aspect, the membrane has an average thickness of from about 2 to 10 mm (or 2, 3, 4, 5, 6, 7, 8, 9 or 10 mm). In yet another aspect, the membrane is contact with an aqueous layer.

In another aspect, the biocompatible polymer having the inorganic carbon and algae is in the form of beads having an average diameter of from 0.1 to 10 mm. In a related aspect, the beads are suspended in an aqueous solution. In another aspect, the algae is encapsulated by the biocompatible polymer. In a related aspect, the algae is partially encapsulated by the biocompatible polymer. In another aspect, the algae is partially encapsulated and partially on the outside of the surface of the biocompatible polymer.

A. Biocompatible Polymer

Immobilization of microalgae cells refers to various techniques, such as covalent/affinity binding, physical adsorption, semi-permeable membrane confinement, and gel encapsulation.

Gel encapsulation of algae provides direct benefits to microalgae growth, like protection of microalgae from natural predators and reducing competition of nutrients from other microbes. Recent research also reveals that this method may promote the growth of microalgae, promote increased chlorophyll content compared to free cells, indicating better photosynthesis efficiency and a potential for high energy yields. Higher yields of glycerol have been reported on immobilized microalgae than their free-living counterparts, which is encouraging for microalgae oil production. Meanwhile, the choice of hydrogel avoids the toxicity of other alternative materials, such as polyurethane or silicate, to the microalgae. The impact of immobilization on the morphology and metabolism of microalgae has been proved to be minor or negligible.

Novel designs and synthesis techniques for integrated microalgae cultivation systems using bio-compatible hydrogel matrices with the aim of solving the problems of CO₂ supply, nutrient distribution, water loss and biomass harvesting are needed. The present disclosure provides methods for entrapping and dispersing carbon dioxide gas into a hydrogel matrix, which also contains necessary nutrients for microalgae growth. Microalgae cells may be immobilized inside or on the surface of the hydrogel matrix. Further, hydrogels of the instant disclosure may contain the same number of microalgae cells found throughout the depth of water in open ponds and can be made into convenient geometries including, thin plates (few millimeters thick), beads (few millimeters in diameters) and cylinders.

The embodiments of this disclosure provide method and techniques for cultivating algae at cell densities of an open pond system but using significantly much less water than the open pond system; combining both CO₂ capture and delivery via a hydrogel matrix for direct application in a microalgae open-pond system; combining both nutrient feeding and CO₂ delivery via a hydrogel matrix; providing controlled CO₂ delivery and uniform nutrient distribution to microalgae growth via a hydrogel matrix; providing protection against environmental contamination by unwanted algae species and other harmful organisms, thus permitting ideal and select microalgae growth conditions; and reducing aerosolization and the unwanted spread in of algae cells in the environment.

Additional benefits conferred by the methods and systems of biocompatible polymers disclosed herein include a multifunctional platform for microalgae cultivation and carbon sequestration; reversible (sol-gel) hydrogel so that the polymers are recovered and reused; universal substrate for integrating other elements necessary to improve microalgae productivity; controlled delivery of CO₂ by pockets inside hydrogel matrix, which reduce direct loss back into the atmosphere; uniform delivery of nutrients embedded inside the hydrogel matrix overcome the problems of nutrient “dead-zones” (or poor mixing); hydrogel matrix floating on top of and open pond system functions as a cover to reduce water evaporation; algae cells encapsulated in floating hydrogel beads facilitate harvesting of biomass; generally, the methods and systems may be applied to scale-up and application to current open pond cultivation systems; and biocompatible polymers are microalgae and environmentally friendly, and may be recycled along with microalgae by-products.

Polymers which may be used in the present invention include, but are not limited to, one or more of the polymers selected from the group consisting of poly(vinyl alcohol), polyacrylamide, poly (N-vinyl pyrolidone), poly(ethylene oxide) (PEO), hydrolysed polyacrylonitrile, polyacrylic acid, polymethacrylic acid, poly(hydroxyethyl methacrylate), polyurethane polyethylene amine, poly(ethylene glycol) (PEG), cellulose, cellulose acetate, carboxy methyl cellulose, alginic acid, pectinic acid, hyaluronic acid, heparin, heparin sulfate, chitosan, carboxymethyl chitosan, chitin, collagen, pullulan, gellan, xanthan, carboxymethyl dextran, chondroitin sulfate, cationic guar, cationic starch as well as salts and esters thereof.

The polymers of biocompatible polymer (e.g., hydrogel), may also comprise polymers of two or more distinct monomers. Monomers used to create copolymers for use in the matrices include, but are not limited to acrylate, methacrylate, methacrylic acid, alkylacrylates, phenylacrylates, hydroxyalkylacrylates, hydroxyalkylmethacrylates, aminoalkylacrylates, aminoalkylmethacrylates, alkyl quaternary salts of aminoalkylacrylamides, alkyl quaternary salts of aminoalkylmethacrylamides, and combinations thereof. Polymer components may include blends of other polymers.

In one aspect, the disclosure provides a hydrogel of copolymers of (hydroxyethyl methacrylate) and methacrylic acid. In another aspect, the hydrogel comprises a binding molecule and a matrix hydrogel of copolymers of (hydroxyethyl methacrylate), methacrylic acid, and alkyl quaternary salts of methacrylamides.

The polymers may be modified to contain nucleophilic or electrophilic groups. The polymers may further comprise polyfunctional small molecules that do not contain repeating monomer units but are polyfunctional, (i.e., containing two or more nucleophilic or electrophilic functional groups). These polyfunctional groups may readily be incorporated into conventional polymers by multiple covalent bond-forming reactions. For example, PEG can be modified to contain one or more amino groups to provide a nucleophilic group. Examples of other polymers that contain one or more nucleophilic groups include, but are not limited to, polyamines such as ethylenediamine, tetramethylenedianiine, pentamethylenediamine, hexamethylenediamine, δw-(2-hydroxyethyl)amine, ow-(2-aminoethyl)amine, and trø-(2-aminoethyl)amine.

B. Engineered Algae

Methods for the transformation of various types of algae are known to those skilled in the art. See for example Radakovits et al., Eukaryotic Cell, 9, 486-501 (2010), which is incorporated herein by reference. The transformation of the chloroplast genome was the earliest method and is well documented in the literature (Kindle et al., Proc Natl Acad Sci., 88, p. 1721-1725 (1991)). A variety of methods have been used to transfer DNA into microalgal cells, including but not limited to agitation in the presence of glass beads or silicon carbide whiskers, electroporation, biolistic microparticle bombardment, and Agrobacterium tumefaciens-mediated gene transfer. A preferred method of transformation for the present invention is biolistic microparticle bombardment, carried out with a device referred to as a “gene gun.”

Different regions of the alga may be targeted for transformation in different embodiments of the invention. Target regions include the nuclear genome, the mitochondrial genome, and the chloroplast genome. The preferred target region can vary depending on the gene being expressed. For example, if an alga has been modified to express a lethal gene that is obtained from a bacterium, it may be preferable to express the lethal gene in the chloroplast or mitochondrion, as these organelles evolved from bacteria and retain many similarities. This can be achieved using a chloroplast expression vector that employs 2 intergenic regions of the chloroplast genome that flank and drive the site-specific integration of a transgene cassette (5′ untranslated region, or 5′ UTR followed by the coding sequence of the protein to be expressed which can drive the biological function desired, followed by a 3′ UTR). The 5′ UTR contains a cis acting site that allows docking of the RNA polymerase that drives transcription of the transgene. The 3′ UTR contains sequence that allows for the correct termination of the transcription by RNA polymerase. However, in other cases, such as when the essential or lethal gene has an effect in various regions of the cell, it may be preferable to express the gene in the nucleus if the algae is eukaryotic. This can be achieved with a gene cassette that employs a eukaryotic promoter sequence upstream of the protein coding sequence and a eukaryotic termination sequence downstream of the protein coding sequence.

Genetically modified algae can be transformed to include an expression cassette. An expression cassette is made up of one or more genes and the sequences controlling their expression. The three main components of a nuclear expression cassette are a promoter sequence, an open reading frame expressing the gene, and a 3′ untranslated region, which may contain a polyadenylation. The cassette is part of vector DNA used for transformation. The promoter is operably linked to the gene expressed represented by the open reading frame.

Single celled microalgae are among the most productive autotrophic organisms in nature due to their high photosynthetic efficiencies and the lack of heterotrophic tissues. Yet, photosynthetic efficiencies and areal productivities are 2 to 3-folds lower than their theoretical potential. This inefficiency is attributed in large part to the poor kinetic coupling between light capture by the light harvesting apparatus and down-stream photochemical and electron transfer processes. During photosynthesis, light captured by the peripheral light-harvesting antenna complexes (LHC) is transferred at nearly 100% efficiency (via quantum coherence processes) to the proximal antenna complexes of the photosystem II (PSII) and photosystem I (PSI) reaction center (RC) complexes where the primary charge separation occurs. Wild-type (WT) algae typically possess large PSII peripheral antennae complexes (LHCII), which maximize light capture at both high and limiting light intensities. However, light harvesting antenna size is not optimized for achieving maximal apparent quantum efficiency in monocultures where competition for light between different species is absent. In nearly all photosynthetic organisms, photosynthesis light saturates at ˜25% of the full sunlight intensity. This is due to the fact that at saturating light intensities, the rate of photon capture substantially (>100×) exceeds the rate of linear photosynthetic electron transfer resulting in a large fraction of the captured light energy being dissipated as heat or fluorescence by non-photochemical quenching (NPQ) processes. These dissipative energy losses account for the greatest inefficiencies (˜50%) in the conversion of light into chemical energy during photosynthesis. Since light is a resource for photosynthetic organisms, it is expected that competition for this resource drives the evolution of antennae size. Ironically, having large, inefficient antennae may increase evolutionary fitness since organisms that compete better for light effectively shade those that are less efficient at capturing light. In mixed species communities, being best at capturing light may be a selective advantage but in monocultures being more efficient at light utilization (energy conversion) may be the better fitness or growth strategy.

To date, the most effective strategy to increase photosynthetic light utilization efficiency is to reduce the size of the light-harvesting antenna per RC complex. By reducing the effective optical cross section of the antennae complexes the probability of saturating electron transfer at full sunlight intensities is reduced. Significantly, a reduction in antennae size/RC is also predicted to reduce cell shading and increase the penetration of photosynthetically active radiation to greater depths in the culture water column. In Chlamydomonas reinhardtii, it has been demonstrated that mutants with reduced antenna size can be generated by eliminating chlorophyll (Chl) b synthesis as well as by reducing expression of LHC genes. Previous studies have shown that microalgae lacking the peripheral LHCII have increased photosynthetic rates; however, few studies have demonstrated an increase in growth rate with reduced peripheral antennae size under fully autotrophic growth conditions. To date, nearly all growth studies with algae having altered antennae sizes have been done under mixotrophic (plus acetate) growth conditions.

In addition to harvesting light members of the LHCII gene/protein family also play important roles in; 1) balancing energy distribution between the photosystems (state transitions), 2) regulating cyclic photophosphorylation or ATP synthesis, and 3) mediating the dissipation of excess captured energy as heat through NPQ.

Various methods for improving photosynthetic energy conversion in algae by modulating light harvesting antenna size are known to those of ordinary skill in the art and may be applied to the μPBRs disclosed herein.

For example, the tla3 DNA insertional transformant of Chlamydomonas reinhardtii is a chlorophyll deficient mutant with a lighter green phenotype, and has a lower Chl per cell content and higher Chl a/Chl b ratio than corresponding wild type strains (Kirst H., Plant Physiol., 160(4), pgs 2251-2260(2012)). By a separate method, RNAi constructs were used to simultaneously down-regulate the expression of all 20 genes encoding for LHCI, LHCII, CP26 and CP29 in Chlamydomonas reinhardtii (mutant Stm3) (Mussgnug J., Plant Biotech J., 5(6), pgs. 802-814 (2007)). Further, DNA insertional mutagenesis of Chlamydomonas reinhardtii was employed to isolate tla1, a stable transformant having a truncated light-harvesting chlorophyll antenna size (Polle J., Planta 217 (2003), pgs. 45-59). Moreover, transformation of a permanently active variant NAB1* of the LHC translation repressor NAB1 to reduce antenna size via translation repression was performed. NAB1* expression was demonstrated in Stm6Glc4T7 (T7 strain), leading to a reduction of LHC antenna size by 10-17%. T7 showed a approximately 50% increase of photosynthetic efficiency (PhiPSII) at saturating light intensity compared to the parental strain (Beckman J., J. Biotech, 142 (2009) pgs. 70-77). Further, trans-acting factor (NAB 1) binds to LHCII mRNAs, negatively regulating their translation leading to a reduction of LHCII content under high light growth conditions (Mussgnug, et al., (2005) The Plant Cell 17: 3409-3421). This nucleic acid binding protein 1 (NAB 1) binds to a cold-shock domain consensus sequence (CSDCS) motif found in several LHCII mRNAs, sequestrating them into translationally silent messenger ribonucleoprotein complexes. By inserting the CSDCS element of the LHCMB6 mRNA into the promoter region used to control the expression of the CAO gene, we have created transgenic organisms in which the expression of the CAO gene is modulated in a light dependent manner. At high light intensity the NAB 1 protein binds to its respective mRNA binding site on the engineered CAO transcript, repressing its translation and the synthesis of Chi b, resulting in a reduced PSII peripheral antenna size. Conversely, under lower intensities translational repression by NAB 1 is reduced allowing for increased levels of CAO translation and Chi b synthesis leading to the assembly of wild-type levels of the peripheral PSII antenna and in increased light capture at lower light intensities (see WO/2013/016267).

The present disclosure exploits the ability of certain proteins (redox sensitive modulators) to act as reversible thiol-based redox switches to regulate gene expression in plants and algae to enable the light dependent regulation of PSII antenna size. Such proteins represent a growing family of proteins that is widely dispersed within the plant and animal kingdoms. See generally Antelmann H, & Helmann ID. (2010) Thiol-based redox switches and gene regulation. Antioxid Redox Signal. 2010 Jul. 14. [Epub ahead of print], Brandes et al., (2009) Thiol-based redox switches in eukaryotic proteins. Antioxid Redox Signal. 1 1(5):997-1014, Paget M S, & Buttner M (2003) Thiol-based regulatory switches. Annu Rev Genet. 37:91-121.

Accordingly the term “redox sensitive modulators” refers to the group of proteins capable of mediating the reversible redox dependent regulation of gene transcription or translation. In one aspect such redox sensitive modulators include proteins that include the conserved cold shock domain (Prosite motif PS00352; Bucher and Bairoch, (In) ISMB-94; Proceedings 2nd International Conference on Intelligent Systems for Molecular Biology, Airman R., Brutlag D., Karp P., Lathrop R., Searls D., Eds., pp 53-61, AAAI Press, Menlo Park, 1994; Hofmann et al., Nucleic Acids Res. 27:215, 1999).

The cold shock domain (CSD) is among the most ancient and well conserved nucleic acid binding domains from bacteria to higher animals and plants (Chsikam et al., BMB reports (2010) 43(1) 1-8; Nakaminami et al., (2006) 103(26) 10123-10127). Proteins containing a CSD motif are also referred to as Y box proteins and eukaryotic members of this large family generally contain a secondary auxiliary RNA domain which modulates the RNA affinity of the protein, but can be dispensable for selective RNA recognition.

An exemplary redox sensitive modulator includes the cytosolic RNA binding protein NAB 1 (SEQ ID NO: 9) from Chlamydomonas. NAB 1 harbors 2 RNA binding motifs and one of these motifs, located at the N-terminus, is a cold shock domain. NAB 1 represses the translation of LHCTI (light harvesting complex of photosystem II) by sequesting the encoding mRNAs into translationally silent mRNP complexes. (Mussgnug et al., The Plant Cell (2005) 17 3409-3421).

NAB 1 contains 2 cysteine residues, Cys-181 and Cys-226, within its C-terminal RNA recognition motif. Modification of these cysteines either by oxidation or by alkylation in vitro is accompanied by a decrease in RNA binding affinity for the target mRNA sequence. Recent studies have confirmed that NAB 1 is fully active′ in its dithiol reduced state, and is reversibly deactivated by modification of its cysteines. (Wobbe et al., (2009) Pro. Nat. Acad. Sci. 106(32) 13290-13295).

The term “NAB 1” as used herein includes all naturally-occurring and synthetic forms of NAB 1 that retain redox sensitive modulator activity. Such NAB 1 proteins include the protein from Chlamydomonas, as well as peptides derived from other plant species and genera, and in one aspect algae. In one aspect, “NAB 1” refers to the Chlamydomonas NAB 1 having the amino acid sequence SEQ ID NO: 9.

NAB 1 from a number of different species have been sequenced, and are known in the art to be at least partially functionally interchangeable. It would thus be a routine matter to identify and select a variant being a NAB 1 from a species or genus other than Chlamydomonas. The amino acid sequence of several such variants of NAB 1 (i.e., representative NAB 1 proteins from other species) are shown below.

Chlamydomonas reinhardtii:

(SEQ ID NO: 9)
MGEQLRQQGTVKWFNATKGFGFITPGGGGEDLFVHQTNINSEGFRSLREG
EVVEFEVEAGPDGRSKAVNVTGPGGAAPEGAPRNFRGGGRGRGRARGARG
GYAAAYGYPQMAPVYPGYYFFPADPTGRGRGRGGRGGAMPAMQGVMPGVA
YPGMPMGGVGMEPTGEPSGLQVVVHNLPWSCQWQQLKDHFKEWRVERADV
VYDAWGRSRGFGTVRFTTKEDAATACDKLNNSQIDGRTISVRLDRFA

Chlamydomonas incerta:

(SEQ ID NO: 10)
MGEQLRQQGTVKWFNATKGFGFITPGGGGEDLFVHQTNINSEGFRSLREG
EAVEFEVEAGPDGRSKAVNVTGPAGAAPEGAPRNFRGGGRGRGRARGARG
GYAAAYGYPQMAPVYPGYYFFPADPTGRGRGRGGRGGAMPGMQGVMPGVA
YPGMPMGGVGMEATGDPSGLQVVVHNLPWSCQWQQLKDHFKEWRVERADV
VYDAWGRSRGFGTVRFTTKEDAAMAC

Volvox carteri f. nagariensis:

(SEQ ID NO: 11)
MGEQLRQRGTVKWFNATKGFGFITPEGGGEDFFVHQTNINSDGFRSLREG
EAVEFEVEAGPDGRSKAVSVSGPGGSAPEGAPRNFRGGGRGRGRARGARG
AYAAYGYPQMPPMYPGYYFFPADPTGRGRGRGRGGMPIQGMIQGMPYPGI
PIPGGLEPTGEPSGLQVVVHNLPWSCQWQQLKDHFKEWRVERADVVYDAW
GRSRGFGTVRFATKEDAAQACEKMNNSQIDGRTISVRLDRFE

Physcomitrella patens subsp. Patens:

(SEQ ID NO: 12)
AKETGKVKWFNSSKGFGFITPDKGGEDLFVHQTSIHAEGFRSLREGEVVE
FQVESSEDGRTKALAVTGPGGAFVQGASYRRDGYGGPGRGAGEGGGRGTV
GGAGRGRGRGGRGVGGFVGERSGAAGGERTCYNCGEGGHIARECQNESTG
NARQGGGGGGGNRSCYTCGEAGHLARDC

Zea mays:

(SEQ ID NO: 13)
MAAAARQRGTVKWFNDTKGFGFISPEDGSEDLFVHQSSIKSEGFRSLAEG
EEVEFSVSEGDDGRTKAVDVTGPDGSSASGSRLLHDGAWRPFCIFTSTRQ
PEQHRGSGSDRHDGGDYNHPKPQAIAAGAHSLLLTRACLSSKSPPPSLAV
GLLSVLAQRTGPTPGTTGSAASLSGSSPISLGFNPTSFLPFLQTARWLPC
SDLATSSSSAPSSPPRSLAPSAPPKKALIGASTGSTGIATSSGAGAAMSR
SNWLSRWVSSCSDDAKTAFAAVTVPLLYGSSLAEPKSIPSKSMYPTFDVG
DRILAEKVSYIFRDPEISDIVIFRAPPGLQVYGYSSGDVFIKRVVAKGGD
YVEVRDGKLFVNGVVQDEDFVLEPHNYEMEPVLVPEGYVFVLGDNRNNSF
DSHNWGPLPVRNIVGRSILRYWPPSKINDTIYEPDVSRLTVPSS

Oryza sativa Japonica Group:

(SEQ ID NO: 14)
MASERVKGTVKWFDATKGFGFITPDDGGEDLFVHQSSLKSDGYRSLNDGD
VVEFSVGSGNDGRTKAVDVTAPGGGALTGGSRPSGGGDRGYGGGGGGGRY
GGDRGYGGGGGGYGGGDRGYGGGGGYGGGGGGGSRACYKCGEEGHMARDC
SQGGGGGGGYGGGGGGYRGGGGGGGGGGCYNCGETGHIARECPSKTY

Chlorella variabilis:

(SEQ ID NO: 15)
MAAAKATGTVKWGYGFITPDSGGEDLFVHQTAIVSEGFRSLREGEPVEFF
VETSDDGRQKAVNVTGPNGAAPEGAPRRQFDDGYGAGGGGGSYGGGFGGG
GGGGRRGGGRGGGGYGGGGYGGGYDQGGYGGQPPIACNM

Selaginella moellendorffi:

(SEQ ID NO: 16)
MASPADAKRTGKVKWFNVTKGFGFITPDDGSEELFVHQSAIFAEGFRSLR
EGEIVEFSVEQGEDQRMRAADVTGPDGSHVQGAPSSFGSRGGGGGGGRGG
RGRAGGGDNPIVCYNCNEAGHVSRDCKYQQEGGGGGGGGGGGRGPPSGRR
GGGAGGGSGGGGRGCFTCGAQGHISRDCPSNY

Vitis vinifera:

(SEQ ID NO: 17)
MAQERSTGVVRWFSDQKGFGFITPNEGGEDLFVHQSSIKSDGFRSLGEGE
TVEFQIVLGEDGRTKAVDVTGPDGSSVQGSKRDNYGGGGGGGIASEEIMA
AAAAVVVEEAEAEVVIPAVAVAVVITVVIMGTWLGIALWKAAALVGSVVA
EVEAVEGLVAVAVDATTVDRKGILLENALTLTHRDEGKRGVIVYILFFPA
SSKIFFPV

Triticum aestivum:

(SEQ ID NO: 18)
MGERVKGTVKWFNVTKGFGFISPDDGGEDLFVHQSAIKSDGYRSLNENDA
VEFEIITGDDGRTKASDVTAPGGGALSGGSRPGEGGGDRGGRGGYGGGGG
GYGGGGGGYGGGGGGYGGGGGGYGGGGYGGGGGGGRGCYKCGEDGHISRD
CPQGGGGGGGYGGGGYGGGGGGGRECYKCGEEGHISRDCPQGGGGGGYGG
GGGRGGGGGGGGCFSCGESGHFSRECPNKAH

Cryptosporidium parvum Iowa II:

(SEQ ID NO: 19)
EKPIKLVKMPLSGVCKWFDSTKGFGFITPDDGSEDIFVHQQNIKVEGFRS
LAQDERVEYEIETDDKGRRKAVNVSGPNGAPVKGDRRRGRGRGRGRGMRG
RGRGGRGRGFYQNQNQSQPQSQQQPVSTQSQPVAH

Arabidopsis thaliana:

(SEQ ID NO: 20)
MAMEDQSAARSIGKVSWFSDGKGYGFITPDDGGEELFVHQSSIVSDGFRS
LTLGESVEYEIALGSDGKTKAIEVTAPGGGSLNKKENSSRGSGGNCFNCG
EVGHMAKDCDGGSGGKSFGGGGGRRSGGEGECYMCGDVGHFARDCRQSGG
GNSGGGGGGGRPCYSCGEVGHLAKDCRGGSGGNRYGGGGGRGSGGDGCYM
CGGVGHFARDCRQNGGGNVGGGGSTCYTCGGVGHIAKVCTSKIPSGGGGG
GRACYECGGTGHLARDCDRRGSGSSGGGGGSNKCFICGKEGHFARECTSV
A

Thus all such homologues, orthologs, and naturally-occurring isoforms of NAB 1 from Chlamydomonas as well as other species are included in any of the methods and kits of the invention, as long as they retain detectable activity. It will be understood that for the recombinant production of NAB 1 in different species it will typically be necessary to codon optimize the nucleic acid sequence of the gene for the host organism in question. Such codon optimization can be completed by standard analysis of the preferred codon usage for the host organism in question, and the synthesis of an optimized nucleic acid via standard DNA synthesis.

The NAB 1 may thus include one or more amino acid deletions, additions, insertions, and/or substitutions based on any of the naturally-occurring isoforms of NAB 1. These may be contiguous or non-contiguous. Representative variants may include those having 1 to 8, or more preferably 1 to 4, 1 to 3, or 1 or 2 amino acid substitutions, insertions, and/or deletions as compared to any of sequences listed above.

NAB 1 polypeptides which may be used in any of the methods of the invention may have amino acid sequences which are substantially homologous, or substantially similar to any of the NAB 1 sequences listed above. Alternatively, the NAB 1 may have an amino acid sequence having at least 30% preferably at least 40, 50, 60, 70, 75, 80, 85, 90, 95, 98, or 99% identity with a NAB 1 listed above. In one aspect, the NAB 1 is substantially homologous, or substantially similar to SEQ ID NO: 22.

Fragments of native or synthetic NAB 1 sequences may also have the desirable functional properties of the peptide from which they were derived and may be used in any of the methods of the invention. The term “fragment” as used herein thus includes fragments of NAB 1 provided that the fragment retains the biological activity of the whole molecule. The fragment may also include an N-terminal or C-terminal fragment of NAB 1. Preferred fragments comprise residues 1-80 of native NAB 1, comprising the cold shock domain, or residues 160 to 247 comprising the RNA recognition motif. Also included are fragments having N- and/or C-terminal extensions or flanking sequences. The length of such extended peptides may vary, but typically are not more than 50, 30, 25, or 20 amino acids in length.

Fusion proteins of NAB 1, and fragments of NAB 1 to other proteins are also included, and these fusion proteins may enhance NAB 1's biological activity, targeting, binding or redox sensitivity. It will be appreciated that a flexible molecular linker (or spacer) optionally may be interposed between, and covalently join, the NAE 1 and any of the fusion proteins disclosed herein. Any such fusion protein many be used in any of the methods of the present invention.

Variants may include, e.g., different allelic variants as they appear in nature, e.g., in other species or due to geographical variation. All such variants, derivatives, fusion proteins, or fragments of NAB 1 are included, may be used in any of the methods claims disclosed herein, and are subsumed under the term “NAB 1”.

The variants, derivatives, and fragments are functionally equivalent in that they have detectable redox dependent RNA binding activity. More particularly, they exhibit at least 40%, preferably at least 60%, more preferably at least 80% of the activity of wild type NAB 1, particularly Chlamydomonas NAB 1. Thus they are capable of functioning as NAB 1, i.e., can substitute for NAB 1 itself.

Such activity means any activity exhibited by a native NAB 1, whether a physiological response exhibited in an in vivo or in vitro test system, or any biological activity or reaction mediated by a native NAB 1 e.g., in an enzyme assay or in binding to test tissues, nucleic acids, or metal ions.

Exemplary chlorophyll A oxygenase nucleic acid sequences can be used to prepare expression cassettes useful for inhibiting or suppressing chlorophyll A oxygenase expression, and for providing for heterologous recombinant CAO genes, are listed above (see. A number of methods can be used to inhibit gene expression in plants. For instance, siRNA, antisense, or ribozyme technology can be conveniently used. For example, in Chlamydomonas, antisense inhibition can be used to decrease expression of a targeted gene (e.g., Schroda, et al (1999) Plant Cell 11(6):165-178). Alternatively, an RNA interference construct can be used (e.g., Schroda, et al., (2006) Curr Genet. 49:69-84).

For antisense expression, a nucleic acid segment from the desired chlorophyll A oxygenase gene is cloned and operably linked to a promoter such that the antisense strand of RNA will be transcribed. The expression cassette is then transformed into plants, e.g., algae, and the antisense strand of RNA is produced. The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of chlorophyll A oxygenase can be useful for producing a plant in which chlorophyll A oxygenase expression is suppressed. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred. Sequences can also be longer, e.g., 1000 or 2000 nucleotides are greater in length.

Catalytic RNA molecules or ribozymes can also be used to inhibit expression of chlorophyll A oxygenase genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs.

A number of classes of ribozymes have been identified. One class of ribozymes is derived from a number of small circular RNAs that are capable of self-cleavage and replication in plants. Ribozymes, e.g., Group I introns, have also been identified in the chloroplast of green algae (see, e.g., Cech et al., (1990) Annu Rev Biochem 59:543-568; Bhattacharya et al., (1996) Molec Biol and Evol 13:978-989; Erin, et al., (2003) Amer J Botany 90:628-633; Turmel, et al., (1993) Nucl Acids Res. 21:5242-5250; and Van Oppen et al., (1993) Molec Biol and Evol 10: 1317-1326). The design and use of target RNA-specific ribozymes is described, e.g., in Haseloff et al. (1 88) Nature, 334:585-591.

Another method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., (1990) The Plant Cell 2:279-289; Flavell, (1994) Proc. Natl. Acad. Sci., USA 91:3490-3496; Kooter and Mol, (1993) Current Opin. Biol. 4: 166-171; and U.S. Pat. Nos. 5,034,323, 5,231,020, and 5,283, 184

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 90% or 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are over-expressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Endogenous gene expression may also be suppressed by means of RNA interference (RNAi), which uses a double-stranded RNA having a sequence identical or similar to the sequence of the target chlorophyll A oxygenase gene. See generally, PCT International Publication Nos. WO 99/32619 WO 99/07409, WO 00/44914. WO 00/44895, WO 00/63364 WO 00/01846, WO 01/36646, WO 01/75164, WO 01/29058, WO 02/055692, WO 02/44321, WO2005/054439, and WO2005/110068.

Non-limiting examples of algae species that can be used with the compositions and methods described herein include for example, Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tenia, Chlorella vulgaris var. autotrophica, Chlorella vulgaris var. viridis, Chlorella vulgaris var. vulgaris, Chlorella vulgaris var. vulgaris fo. tenia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Chlamydomonas moewusii Chlamydomonas reinhardtii Chlamydomonas sp. Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

C. Light Frequency-Shifting Agents

a. Nanocrystal Quantom Dots (NQD)

Semiconductor nanocrystal quantum dots (NQDs) are desirable fluorophores based on their unique particle-size-tunable optical properties, i.e., efficient and broadband absorption and efficient and narrow-band emission. Further, compared to alternative fluorophores, such as organic dyes, NQDs are characterized by significantly enhanced photostabilility (see U.S. Pat. No. 7,935,419, which is incorporated herein by reference in its entirety). Despite these desirable characteristics, NQD optical properties may be frustratingly sensitive to their surface chemistry and chemical environment. For example, coordinating organic ligands are used to passivate the NQD surface during growth, and are retained following preparation. These coordinating ligands are strong contributors to bulk NQD optical properties such as quantum yields (QYs) in emission; however, the ligands tend to be labile and can become uncoordinated from the NQD surface, and can be damaged by exposure to the light sources used for NQD photoexcitation. Ligand loss through physical separation or photochemistry results in uncontrolled changes in QYs and, in the case of irreversible and complete loss, in permanent “darkening” or photobleaching. In addition, some ligands may be incompatible with certain solvents and systems, thus limiting the uses of a particular NQD.

Furthermore, NQDs are characterized by significant fluorescence intermittency, or “blinking,” at the single NQD level. Without wishing to be limited by theory, blinking is generally considered to arise from an NQD charging process in which an electron (or a hole) is temporarily lost to the surrounding matrix (for example, via Auger ejection or charge tunneling) or captured to surface-related trap states. NQD emission turns “off” when the NQD is charged and turns “on” again when NQD charge neutrality is regained. Blinking is unacceptable for such potential NQD applications as single-photon light sources for quantum informatics and biolabels for real-time monitoring of single biomolecules. Previous attempts to address blinking include the use of charge mediators such as short-chain thiols on the NQD surface. This approach provided at best only a partial, short-term solution however, and encountered such problems as dependence on pH, concentration, lighting conditions, and the NQDs were further incompatible with a number of applications.

It is known that addition of an inorganic shell of a semiconductor material having a higher bandgap can generally enhance QYs and improve stability. See, for example, Hines, M. A.; Guyot-Sionnest, P. J. Phys. Chem. 1996, v. 100, pp. 468-471. However, the optical properties of previously disclosed core/shell and core/multishell NQDs remain susceptible to blinking, photobleaching and ligand issues. A need exists, therefore, for NQDs which have increased stability, and decreased fluorescence intermittency and photobleaching.

The colloidal nanocrystal quantum dots of the present disclosure comprise an inner core and an outer shell. The outer shell comprises an inorganic material, and in one embodiment may consist essentially of an inorganic material. The shape of the colloidal nanocrystal quantum dots may be a sphere, a rod, a disk, and combinations thereof, and with or without faceting. In one embodiment, the colloidal nanocrystal quantum dots include a core of a binary semiconductor material, e.g., a core of the formula MX, where M can be cadmium, zinc, mercury, aluminum, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof. In another embodiment, the colloidal nanocrystal quantum dots include a core of a ternary semiconductor material, e.g., a core of the formula M₁M₂X, where M₁ and M₂ can be cadmium, zinc, mercury, aluminum, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof. In another embodiment, the core of the colloidal nanocrystal quantum dots comprises a quaternary semiconductor material, e.g., of the formula M₁M₂M₃X, where M₁, M₂ and M₃ can be cadmium, zinc, mercury, aluminum, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof. In one embodiment, the core of the colloidal nanocrystal quantum dots comprises silicon or germanium. Non-limiting examples of suitable core materials include cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), aluminum nitride (AlN), aluminum sulfide (AlS), aluminum phosphide (AlP), aluminum arsenide (AlAs), aluminum antimonide (AlSb), lead sulfide (PbS), lead selenide (PbSe), lead telluride (PbTe), gallium arsenide (GaAs), gallium nitride (GaN), gallium phosphide (GaP), gallium antimonide (GaSb), indium arsenide (InAs), indium nitride (InN), indium phosphide (InP), indium antimonide (InSb), thallium arsenide (TlAs), thallium nitride (TlN), thallium phosphide (TlP), thallium antimonide (TlSb), zinc cadmium selenide (ZnCdSe), indium gallium nitride (InGaN), indium gallium arsenide (InGaAs), indium gallium phosphide (InGaP), aluminum indium nitride (AlInN), indium aluminum phosphide (InAlP), indium aluminum arsenide (InAlAs), aluminum gallium arsenide (AlGaAs), aluminum gallium phosphide (AlGaP), aluminum indium gallium arsenide (AlInGaAs), aluminum indium gallium nitride (AlInGaN) and the like, mixtures of such materials, or any other semiconductor or similar materials. In another embodiment, the colloidal nanocrystal quantum dots include a core of a metallic material such as gold (Au), silver (Ag), cobalt (Co), iron (Fe), nickel (Ni), copper (Cu), manganese (Mn), alloys thereof and alloy combinations. Preferably, the core material is selected from the group consisting of GaP, GaAs, GaSb, InP, InAs, InSb, ZnS, ZnSe, ZnTe, CdS, CdSe, CdTe, PbS, PbSe and PbTe.

The NQDs of the present disclosure may comprise at least seven monolayers of inorganic material, which surround the core material and collectively form the outer shell. When the monolayers are comprised of the same material, the NQD is referred to as a thick-shell NQD. When the composition of the individual monolayers varies (but is consistent within the monolayer), the NQD is referred to as a thick multi-shell NQD. In one embodiment, the NQDs of the present disclosure comprise from at least 4 to about 20 monolayers (or 4, 5, 6, 7, 8, 9, 10, 11, 12, 13, 14, 15, 16, 17, 18, 19 or 20), or at least 7 monolayers, alternatively at least 10 monolayers, alternatively at least 12 monolayers, alternatively at least 14 monolayers, alternatively at least 16 monolayers, alternatively at least 19 monolayers, and alternatively comprise from 8 to about 20 monolayers of inorganic material.

In an alternative embodiment, the composition of the monolayers varies gradually, such that the innermost layer consists essentially of material A, and subsequent layers comprise materials A and B in a molar relationship A.sub.1B.sub.1-x, wherein the value of x decreases sequentially from 1 to 0 such that the outermost layer or layers consist essentially of material B. One non-limiting example of such a NQD has the structure Cd.sub.xZn.sub.1-xS, where CdS is material A and ZnS is material B, the inner monolayer consists essentially of CdS and the outer layer(s) consist essentially of ZnS. Such a construction is referred to herein as an “alloyed shell,” and the resulting NQD is referred to as an alloyed NQD. In certain embodiments, the core comprises CdSe and the outer shell comprises CdS.

In other embodiments, the core comprises InP and the outer shell comprises CdS. In related embodiments, the core comprises CuInS₂ and the outer shell comprises ZnS. In a related embodiment, the core comprises InP and the outer shell comprises ZnS. In a related embodiment, the core comprises Zn₃P₂ and the outer shell comprises ZnS. In a related embodiment, the core comprises CuGaS₂ and the outer shell comprises ZnS.

In certain embodiments, population of NQD's used within the biocompatible polymer may all have the same core material and the same outer shell material. In other embodiments, the population of NQD's used within the biocompatible polymer may have different core materials with the same outer shell material. In other embodiments, the population of NQD's used within the biocompatible polymer may have different core materials with the different outer shell materials.

The outer shell may comprise some of the same materials as the core or entirely different materials than the core, and may comprise a semiconductor material. The outer shell may include materials selected from among Group II-VI compounds, Group II-V compounds, Group III-VI compounds, Group III-V compounds, Group IV-VI compounds, Group compounds, Group II-N-V compounds, and Group II-IV-VI compounds. Non-limiting examples of suitable overcoatings include cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), aluminum nitride (AlN), aluminum phosphide (AlP), aluminum arsenide (AlAs), aluminum antimonide (AlSb), gallium arsenide (GaAs); gallium nitride (GaN), gallium phosphide (GaP), gallium antimonide (GaSb), indium arsenide (InAs), indium nitride (InN), indium phosphide (InP), indium antimonide (InSb), thallium arsenide (TlAs), thallium nitride (TlN), thallium phosphide (TlP), thallium antimonide (TlSb), lead sulfide (PbS), lead selenide (PbSe), lead telluride (PbTe), zinc cadmium selenide (ZnCdSe), indium gallium nitride (InGaN), indium gallium arsenide (InGaAs), indium gallium phosphide (InGaP), aluminum indium nitride (AlInN), indium aluminum phosphide (InAlP), indium aluminum arsenide (InAlAs), aluminum gallium arsenide (AlGaAs), aluminum gallium phosphide (AlGaP), aluminum indium gallium arsenide (AlInGaAs), aluminum indium gallium nitride (AlInGaN) and mixtures of any of the above. Preferably, the inorganic material of the outer shell comprises CdS, ZnS, Cd.sub.xZn.sub.1-xS, or combinations thereof.

The number of monolayers will determine the thickness of the outer shell and the diameter of the NQDs. The thickness of the shell must be sufficient to substantially isolate the wavefunction of the NQD core from the NQD surface and surface environment. In one embodiment, the inner core of the NQDs of the present disclosure may have an average diameter of at least 1.5 nm, and alternatively from about 1.5 to about 30 nm (or 1.5, 2, 2.5, 3, 3.5, 4, 4.5, 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5., 26, 26.5, 27, 27.5, 28, 28.5, 29, 29.5, or 30 nm).

The quantum yield for NQDs of the present disclosure is largely independent of chemical environment (e.g., in hexane or in water), and is understood to mean the fraction of the number of emitted photons relative to the number of incident photons.

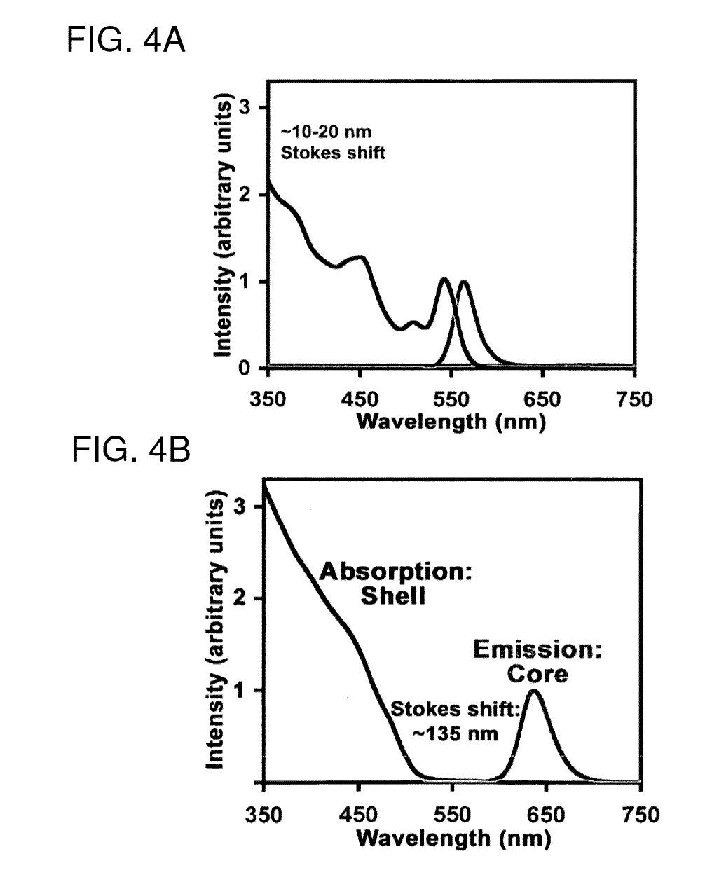

The NQDs of the present disclosure exhibit an enhanced Stokes shift, as depicted in FIG. 4. Thus, the NQDs are characterized by photoluminescence (PL) spectra that are shifted to longer wavelengths (lower energies) compared to previously described NQD cores, and essentially no emission from the shell is observed. FIG. 4a depicts the Stokes shift of previously reported NQDs, whereas FIG. 4b depicts that of the NQDs of the present disclosure. The NQDs of the present disclosure exhibit an effective Stokes shift of at least 75 nm, alternatively of at least 100 nm, and alternatively at least 135 nm.

The NQDs of the present disclosure are ligand-independent, meaning that the exhibited properties remain substantially unchanged regardless of whether ligands are present, and regardless of the identity of the ligand. Some non-limiting examples of ligands include octadecylamine, trioctylphosphine oxide, and/or oleic acid with mercaptosuccinic acid. This ligand-independence provides a significant advantage, in that issues arising from ligand incompatibility with the surrounding environment may be avoided without sacrificing the desirable properties of the NQD. For example, if compatibility with a desired solvent is desired, a solvent-compatible ligand may be substituted. Similarly, if the presence of any ligand is deemed undesirable (for example, in biological systems where a ligand may provoke an immune response), the NQD may be made without ligands. Upon precipitation from growth solution (water) and re-dissolution in hexane, the on-time fractions for NQDs did not change significantly (17% vs. 20% at <0.2 and 54% vs. 49% at >0.8). Further, NQDs precipitated and re-dissolved 7 times and observed no changes in on-time fractions, nor was any significant change observed in on-time fractions upon transfer to water using a standard ligand exchange procedure (i.e., replacing original ligands that are present as a result of the NQD synthesis process, such as octadecylamine, trioctylphosphine oxide, and oleic acid with mercaptosuccinic acid).

Under continuous excitation conditions, significantly suppressed fluorescence intermittence, or blinking behavior, was observed for all NQDs of the present disclosure relative to control samples. The NQDs of the present disclosure are substantially free of both “fast” (about 1-10 ms temporal resolution) and “slow” (about 100-200 ms temporal resolution) blinking behavior. In one embodiment, the on-time fraction is independent of experimental time-resolution over a period of from about 1 ms to about 200 ms, meaning that when the quantum dots are viewed with increased resolution over this time period, the on-time fraction is still at least 0.99. As is typical of traditional NQDs, >70% of the control NQDs have on-time fractions of <0.2, and <5% are non-blinking (i.e., never turn off, at least 0.99 on the x-axis). In contrast, >15% of the NQDs of the present disclosure having at least 7 monolayers are non-blinking and >30% of these NQDs have an on-time fraction of >0.8 (80%) (as illustrated in U.S. Pat. No. 7,935,419, which is incorporated herein by reference in its entirety). These fractions increase as the number of monolayers increases. For example, when the number of monolayers is at least 12, approximately 30% of the NQDs are non-blinking. The absence of blinking behavior (as noted by the intensity not being equal to zero at anytime) over a shorter timescale, i.e. from about 1 ms to about 200 ms, with the inset showing the absence of blinking on a 1 ms timescale was observed. Thus, in one embodiment of the present disclosure, at least 30% of the NQDs have an on-time fraction of about 0.8 or greater, when measured over a period of at least 10 minutes, alternatively from about 5 minutes to about 15 minutes, and alternatively at least 50 minutes, and alternatively at least 1 hour. At least 15%, and alternatively at least 20%, and alternatively at least 30% of the NQDs of the present disclosure have an on-time fraction of about at least 0.99, when measured over a period of at least 10 minutes, alternatively of at least 50 minutes, and alternatively of at least 1 hour.

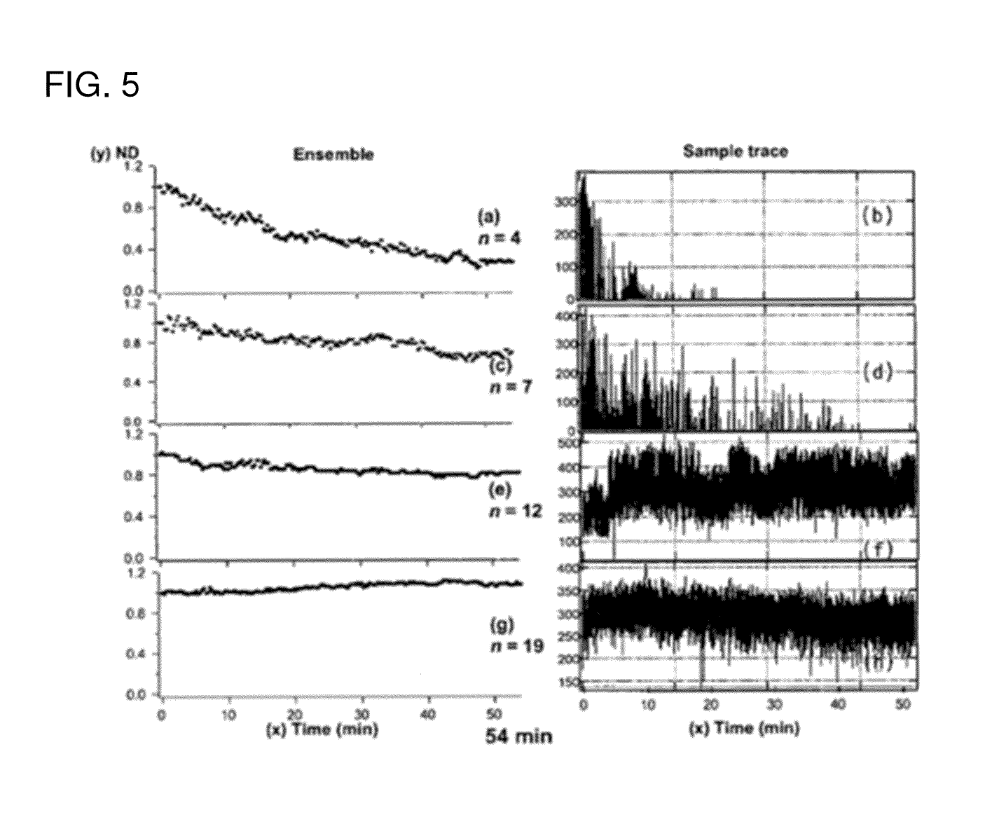

Also importantly, the NQDs of the present disclosure are stable under continuous laser illumination (532 nm, 205 mW laser) at a single dot level, where “stable” herein is understood to mean that an NQD does not exhibit photobleaching (i.e., permanently turning off). The time required for a NQD to exhibit photobleaching is an indicator of the stability of the NQD: the longer the time, the more stable the NQD. This is depicted in FIG. 5. Specifically, samples comprising four monolayers exhibited significant photobleaching (with complete absence of photoluminescence), with only just above 50% still stable after 10 minutes and approximately 30% stable after 1 hr. In contrast, about 90% of the NQDs of the present disclosure having seven monolayers were stable after 10 minutes, and after about 1 hour approximately 80% were still stable. When the number of monolayers is twelve or nineteen, substantially all of the NQDs are stable after 1 hour. Accordingly, in one embodiment about 80% of the NQDs of the present disclosure are stable for a period of at least one hour under continuous laser illumination as defined herein, and alternatively substantially all (100%) of the NQDs of the present disclosure are stable for a period of at least 1 hour. In another embodiment, about 90% of the NQDs of the present disclosure are stable for a period of at least ten minutes under continuous laser illumination as defined herein, and alternatively substantially all (100%) of the NQDs of the present disclosure are stable for a period of at least 10 minutes.

Unlike conventional NQDs, the NQDs of the present disclosure may exhibit multi-exciton emission when pumped with sufficiently high pump power. In other words, the NQDs of the present disclosure are capable of emitting multiple photons of different energies simultaneously. As each energy is characteristic of a different emission color, the NQDs of the present disclosure emit more than one color of light simultaneously. The multiple emissions result from “multi-exciton states,” such as bi-exciton states and tri-exciton states. In conventional NQDs, emission from any state other than a simple single exciton state is quenched due to an ultrafast non-radiative exciton recombination process, known as Auger recombination. Further, conventional NQDs also suffer from photodegradation at very high pump powers. The emergence of new emission colors (peaks) at about 2.10-2.15 eV, in NQDs having 16 monolayers of CdS, with increasing pump power (the power increases with decreasing values of y on the y-axis) was observed. These emissions are made possible by the unique nanoscale architecture of the NQDs of the present disclosure, at energies higher than the energy of normal single exciton emission. In one embodiment, the multiexciton states of the quantum dots emit photons at a pump power of from about 400 W/cm.sup.2 to about 40 kW/cm.sup.2, and alternatively at a pump power of from about 400 W/cm.sup.2 to about 20 kW/cm.sup.2, wherein the pump is understood to be a 532 nm pump. Multi-exciton emission may occur at essentially any temperature, and in one embodiment occurs at 300K. In one embodiment, the NQDs of the present disclosure exhibit multiexciton states which emit two photons, and alternatively three photons, and alternatively at least three photons.

b. Fluorescence Protein

Green Fluorescent Protein (GFP) from the hydromedusa Aequorea aequoreal Aequorea victoria (A. victoria) was identified by Johnson et al., J. Cell Comp. Physiol. (1962) 60:85-104 as a secondary emitter of the jellyfish's bioluminescent system, transforming blue light from the photoprotein aequorin into green light. The cDNA encoding A. victoria GFP (avGFP) was cloned as reported in Prasher et al., Gene (1992) 111:229-233. When ectopically expressed, this gene will produce a fluorescent protein due to its unique ability to independently form a chromophore (Chalfie et al., Photochem Photobiol (1995) 62:651-656). This finding has enabled broad applications for the use of GFP in cell biology as a genetically encoded fluorescent label.

Genes encoding fluorescent proteins have since been cloned from organisms of a wide variety of different phylogenetic clades including, but not limited to: Hydrozoa, Anthozoa, Arthropoda (Copepoda) and Chordrata (Brachiostoma), e.g., as reported in: Matz et al., Nat. Biotechnol. (1999) 17: 969-973; Chudakov et al., Trends Biotechnol. (2005) 23: 605-613; Shagin et al., MoI. Biol. Evol. (2004) 21: 841-850; Masuda et. al., Gene (2006) 372: 18-25; Deheyn et al., Biol. Bull. (2007) 213: 95-100; and Baumann et al., Biol. Direct. (2008) 3: 28. Currently, the fluorescent protein (FP) family (also referred to in the art as the “GFP family”) includes hundreds of member proteins. While these proteins may collectively be referred to as members of the “GFP family”, emission maxima may vary widely in terms of wavelength, and therefore not all members of the family fluoresce green.

Proteins of the GFP family share a common GFP-like domain. This domain can be easily identified in the amino acid sequences of the various family members using available software for the analysis of protein domain organization, e.g., by using the Conserved Domain Database (CDD) program available at the website formed by placed “http://www.” in front of “ncbi.nlm.nih.gov/Structure/cdd/” and the Simple Modular Architecture Research Tool (SMART) program available at the website formed by placing “http://smart.” in front of “embl-heidelberg.de/”. For example, the GFP-like domain of avGFP begins at amino acid residue 6 and ends at amino acid residue 229. It has been demonstrated that a core domain within this domain, the “minimum GFP-like domain,” produced by truncating the protein at the N-terminus (up to 9 amino acid residues) and C-terminus (up to 11 amino acid residues) is sufficient to provide for maturation and fluorescence of GFP family proteins (Shimozono et al., Biochemistry. 2006; 45(20): 6267-71). Thus, when expressed, both GFP-like domain polypeptides and minimum GFP-like domain polypeptides can produce a protein that exhibits fluorescence.

In red GFP-like proteins, additional chemical modification of the GFP-like chromophore occurs. In particular, oxidation of a Ca—N bond at residue 65 (avGFP numbering) results in an acylimine group conjugated to a GFP-like core in DsRed (see Gross et al., Proc. Nat'l Acad. Sci USA (2000) 97:1 1990-1 1995; Wall et al., Nat. Struct. Biol. (2000) 7:1 133-1 138; and Yarbrough et al., Proc. Nat'l Acad. Sci. USA (2001) 98:462-467). The DsRed-like chromophore is formed within many other proteins with red-shifted absorption and fluorescence (See e.g., Pakhomov, A. A. and Martynov, V. I., Chem. Biol. (2008) 15: 755-764). In some proteins, the acylimine moiety of the DsRed chromophore is further attacked by various nucleophiles to form additional types of red-shifted chromophores. For example, the chromophore in the purple chromoprotein asFP595 is formed by hydrolysis of the acylimine group, resulting in cleavage of the protein backbone and formation of a keto group conjugated to a GFP-like chromophore core (see e.g., Quillin et. al., Biochemistry (2005) 44: 5774-5787; and Yampolsky et al., Biochemistry (2005) 44: 5788-5793). In the orange fluorescent proteins mOrange and mKO, nucleophilic addition of Thr65 (in mOrange) or Cys65 (in mKO) side chain groups leads to unusual heterocycles without protein backbone scission (see e.g., Shu et al., Biochemistry (2006) 45: 9639-9647 and Kikuchi et al., Biochemistry (2008) 47: 1 1573-1 1580). Thus, amino acid substitution of one or more residues in the chromophore and chromophore environment will strongly affect fluorescence maxima of FPs. These positions crucial for fluorescence of particular color can be found by sequence comparison of fluorescent proteins of different colors. In many cases, one amino acid substitution, i.e. corresponding to residue 65 of avGFP, is required to produce a green fluorescent protein from the red FP (see e.g., Gurskaya et al., BMC Biochemistry (2001) 2:6).

Among far-red fluorescent proteins developed to date, mKate2 is the brightest one, and demonstrates advantageous characteristics including high pH stability, photostability, and fast maturation (Shcherbo et al., Biochem J. 2009; 418(3): 567-74). mKate2 was produced on the basis of Entacmaea quadhcolor EqFP578 protein (U.S. Pat. No. 7,638,615) and comprises several amino acid substitutions altering its hydrophobic and hydrophilic interfaces. mKate2 has the following spectral and biochemical characteristics: excitation maximum 588 nm; emission maximum 633 nm, quantum yield 0.4 (at pH 7.5), extinction coefficient 62,500 M^{˜1}cm^{˜1} (at pH 7.5), calculated brightness 25.0 (product of extinction coefficient and quantum yield, divided by 1000), and pKa 5.4 (Shcherbo et al., Biochem J. 2009; 418(3): 567-74).

mKate2 behaves as monomer in gel filtration (size exclusion) performed using low pressure liquid chromatography (LPLC), as reported by Shcherbo et al. (Shcherbo et al., Biochem J. 2009; 418(3): 567-74). However, mKate2 is capable of dimerization, which can be detected using gel filtration (size exclusion chromatography) performed using fast protein liquid chromatography (FPLC). This dimerization can alter the activity of proteins of interest that are fused to mKate2.

The applications of interest include the use of the subject proteins in fluorescence resonance energy transfer (FRET) methods. In these methods, the subject proteins serve as donor and/or acceptors in combination with a second fluorescent protein or dye, for example, a fluorescent protein as described in Matz et al., Nature Biotechnology 17:969-973 (1999); a mutants of green fluorescent protein from Aequorea victoria, for example, as described in U.S. Pat. Nos. 6,066,476; 6,020,192; 5,985,577; 5,976,796; 5,968,750; 5,968,738; 5,958,713; 5,919,445; 5,874,304, the disclosures of which are herein incorporated by reference; other fluorescent dyes such as coumarin and its derivatives, 7-amino-4-methylcoumarin and aminocoumarin; bodipy dyes; cascade blue; or fluorescein and its derivatives, such as fluorescein isothiocyanate and Oregon green; rhodamine dyes such as Texas red, tetramethylrhodamine, eosins and erythrosins; cyanine dyes such as Cy3 and Cy5; macrocyclic chealates of lenthaninde ions, such as quantum dye; and chemilumescent dyes such as luciferases, including those described in U.S. Pat. Nos. 5,843,746; 5,700,673; 5,674,713; 5,618,722; 5,418,155; 5,330,906; 5,229,285; 5,221,623; 5, 182,202; the disclosures of which are herein incorporated by reference.

The amino acid sequences of exemplary fluorescent proteins that may be used in the context of the μPBRs disclosed herein are as follows:

Katushka 9-5:
(SEQ ID NO: 21)
MGEDSELISENMHMKLYMEGTVNDHHFKCTSEGEGKPYEGTQTMKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEASTEML
YPADSGLRGHSQMALKLVGGGYLHCSLKTTYRSKKPAKNLKMPGFYFVDR
KLERIKEADKETYVEQHEMAVARYCDLPSKLGHSNPQRSTVWY
Kat650-21
(SEQ ID NO: 22)
MGEDSELISENMHMKLYMEGTVNGHHFKCTSEGEGKPYEGTQTAKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEASTEML
YPADSGLRGHSQMALKLVGGGYLHCSLKTTYRSKKPAKNLKMPGFYFVDR
KLERIKEADKETYVEQHEMAVARYCDLPSKLGHS
Kat670-23
(SEQ ID NO: 23)
MGEDSELISENMHTKLYMEGTVNGHHFKCTSEGEGKPYEGTQTCKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEANTEML
YPADSGLRGHNQMALKLVGGGYLHCSLKTTYRSKKPAKNLKMPGFYFVDR
KLERIKEADKETYVEQHEMAVARYCDLPSKLGHS
KatX1
(SEQ ID NO: 24)
MGEDSELISENMHTKEYMEGTVNGHHFKCTSEGEGKPYEGTQTCKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEANTEML
YPADSGLRGHNQMALKLVGGGYLHCSLKTTYRSKKPAKNLKMPGFYFVDR
KLERIKEADKETYVEQHEMAVARYCDLPSKLGHS
KatX2
(SEQ ID NO: 25)
MGEDSELISENMHSKEYMEGTVNGHHFKCTSEGEGKPYEGTQTAKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEASTEML
YPADSGLRGHSQMALKLVGGGYLHCSLKTTYRSKKPAKNLKMPGFYFVDR
KLERIKEADKETYVEQHEMAVARYCDLPSKLGHS
Katusha9-5A
(SEQ ID NO: 26)
MGEDSELISENMHMKLYMEGTVNDHHFKCTSEGEGKPYEGTQTMKIKVVE
GGPLPFAFDILATSFMYGSKTFINHTQGIPDFFKQSFPEGFTWERITTYE
DGGVLTATQDTSLQNGCLIYNVKINGVNFPSNGPVMQKKTLGWEASTEML
YPADSGLRGHAQMALKLVGGGYLHCSLKTTYRSKKPAKNLKMPGFYFVDR
KLERIKEADKETYVEQHEMAVARYCDLPSKLGHS

D. Exogenous Agent for Conversion of Carbon Dioxide to Bicarbonate

Carbonic anhydrases (CA, EC 4.2.1.1) are ubiquitous enzymes that catalyze the reversible hydration/dehydration of carbon dioxide/bicarbonate. As such, there is an increasing interest in exploiting CA in algae as a way to capture CO₂ and convert it into biofuels or other valuable products (Fulke et. al., 2010; Ramanan et al., 2010). Human carbonic anhydraseII (HCA II) is a suitable candidate for these applications: It is easy and cost-effective to express and purify, from overexpression in Escherichia coli; it has fast kinetic parameters, with a turnover rate of 10⁶ s⁻¹; it is very soluble, to concentrations of <100 mg/ml; and it has an intermediate melting temperature, TM of ˜58° C. (Avvaru et al., 2009). Also, from a rational design and bio-engineering perspective much is known about the structure and catalytic mechanism of this enzyme. However, for industrial applications, small ‘improvements’ in stability, without detriment to yield, activity or solubility can add greatly in the development of HCA II as a better bio-catalyst, as the environment of action may be at an extreme pH and/or elevated temperature. Use of the free enzyme in solution has also many serious drawbacks, such as low stability that limits re-usability, recovery and cost in an industrial setting (Kanbar and Ozdemir, 2010). Having a stable HCA II variant with wild-type kinetic features will be essential for industrial applications—immobilized or in solution—in carbon sequestration and/or biofuel production as it will help limit costs.

The HCA II isozyme is the best-characterized CA to date. It is a monomeric Zn containing metalloenzyme with a molecular weight of ˜29 kDa. It is classified as an ultra-fast enzyme with a k_{cat}/KM of 1.5×10⁸M⁻¹ s^{-l} and a k, of 1.4×10⁶ s⁻¹ and among the fastest CA isozyme characterized so far. However, the production of thermostabilized enzymes is still a significant challenge and there are many approaches to this, each with varying success. One popular strategy is to create large libraries of mutants through random mutagenesis and directed evolution while selecting for a specific criteria (Jochens et al., 2010). Other studies have focused on a more rational approach with a small set of targeted changes, like introducing Arg residues as stabilizing elements (Mrabet et al., 1992). Unlike improving substrate-binding or enzyme kinetic properties, protein stability is a function of many variables, from protein folding, core packing, surface electrostatics, to overall rigidity and it appears that these determinants have varying importance in different proteins (Filikov et al., 2002; Permyakov et al., 2005; Strickler et al., 2006). To address these uncertainties computational tools have been developed that can assist with rational thermostability design, and while some of these methods are informative they do not suggest a generalizable strategy that will work for all proteins (Filikov et al., 2002; Potapov et al., 2009). Another successful approach is known as the B-factor iterative test principle method where areas in a protein with high thermal fluctuations are identified from the X-ray crystal structures. These areas are then subjected to iterative rounds of mutagenesis while selecting for thermostability (Reetz et al., 2006). The development of rational design approaches based on specific crystallographic data that inform on surface electrostatics, hydrophobic interactions as well as hydration and H-bonding are appealing because this can lead to the development of guidelines for thermostabilization of all proteins. Owing to the complex nature of protein folding, kinetics and stability, the most effective strategy will likely be a combination of many techniques, both computational and experimental.

The following examples are provided to illustrate certain particular features and/or embodiments. These examples should not be construed to limit the disclosure to the particular features or embodiments described.

### EXAMPLES

### Example 1

### Preparation of Biocompatible Polymer for Algae Cultivation

This example provides methods for making a biocompatible polymer (e.g., alginate hydrogel) having inorganic carbon (e.g, carbon dioxide) and algae.

Two approaches were taken to prepare hydrogel foam as storage for CO₂ and a feed and growth support for microalgae. One approach used gaseous CO₂ as an input, and the other approach used carbonates as carbon source. For both approaches, CO₂ gas was successfully captured and stored inside hydrogel beads having an average diameter of from about 0.2 mm to about 5 mm. Either approach may be scaled-up based on the defined parameters provided below.

Nutrient rich media for algae cultivation is known in the art. By way of example, nutrient rich media (High Salt Media or HS Media) was made and added to the biocompatible polymer solutions described below to promote algae cultivation in the biocompatible polymer. Briefly, a 1 L stock solution (B Solution) was prepared by adding the following components and quantities in water to 1 L:

| B solution | 1 L | |
|---|---|---|
| NH₄Cl | 100 | g |
| MgSO₄•7H₂O | 4.0 | g |
| CaCl₂•2H₂O | 2.0 | g |

A second 1 L stock solution (Phosphate Solution) was prepared by adding the following components and quantities in water to 1 L:

| Phosphate solution | 1L | |
|---|---|---|
| K₂HPO₄ | 288 | g |
| KH₂PO₄ | 144 | g |

In water, 5 mL of B Solution was combined with 5 mL of Phosphate Solution and 5 mL Hutner's Solution.

### Approach 1: Direct Capture

For this method, a T-junction apparatus was used to impinge a stream of carbon dioxide gas at approximately 5 psi on a stream of aqueous alginic sodium (Sigma-Aldrich®) solution. The aqueous solution may alternatively contain (1% w/w) surfactants sodium dodecyl sulfate (SDS) 20 mg/L. The gas stream of CO₂ and the aqueous stream of alginate solution mixed to form CO₂ bubbles in the alginate solution. The CO₂ and alginate mixture was collected and rapidly cross-linked in a CaCl₂ solution to form a hydrogel foam with approximately 40% by volume CO₂. The pockets of CO₂ gas inside the hydrogel foam were relatively uniform throughout the hydrogel, and the average diameter of the individual pockets of CO₂ gas ranged from about 0.5 mm to about 5 mm (or 0.5, 1, 1.5, 2, 2.5, 3, 3.5, 4, 4.5 and 5 mm) depending on the flow rate used during the impinging process. Flow-rate adjustment also allowed for controlling the dispersity of the CO₂ gas pockets throughout the hydrogel.

### Approach 1: Carbonate Decomposition

For this method, two solutions were made and then mixed. One solution was an aqueous alginic solution prepared by dissolving 0.15 g alginic sodium (Sigma-Aldrich®) powder in 15 mL 0.2M sodium bicarbonate aqueous (EMD™) solution. The mixture was heated to 50° C. and stirred for approximately 10 to 15 minutes. A second solution was an aqueous cross-linking solution prepared by dissolving 0.333 g calcium chloride (EMD™) powder in 15 mL (Kroger®) distilled white vinegar (approximately 5% acidic content, at pH 2). The aqueous alginic solution was combined with the aqueous cross-linking solution at a 1:1 ratio and mildly stirred. A hydrogel foam formed in approximately 30 seconds and had approximately 50% by volume CO₂. The pH of the resultant hydrogel foam was about 4.5. The hydrogel foam was stable for over a one week time period when placed in contact with a thin film of water. The density of the hydrogel foam is equivalent to or slightly less than that water depending on the amount of entrapped CO₂ gas. The density differential allowed the hydrogel foam to float or remain buoyant at all times. The average diameter of the individual pockets of CO₂ gas in the hydrogel foam ranged from about 1 mm to about 5 mm (or 1, 1.5, 2, 2.5, 3, 3.5, 4, 4.5 and 5 mm).

The above two methods for making hydrogel foams can be adapted for making hydrogels of any desired form or shape, for example, a membrane, beads or cylindrical. One of ordinary skill in the art could adapt the above methods to modify the size and density of the CO₂ gas pockets in the hydrogel. For example, one of ordinary skill in the art could change the flow rates of the aqueous stream and/or gaseous stream in the Direct Capture method, or change the amount of sodium bicarbonate in the aqueous solution in the Carbonate Deposition method.

Further, the hydrogel polymers described above may be reused by depolymerization of the hydrogel by increasing the pH to extract the cross-linker. The resultant product was reused to regenerate a hydrogel at about pH 7 to 8.

### Algae Hydrogel Cultivation:

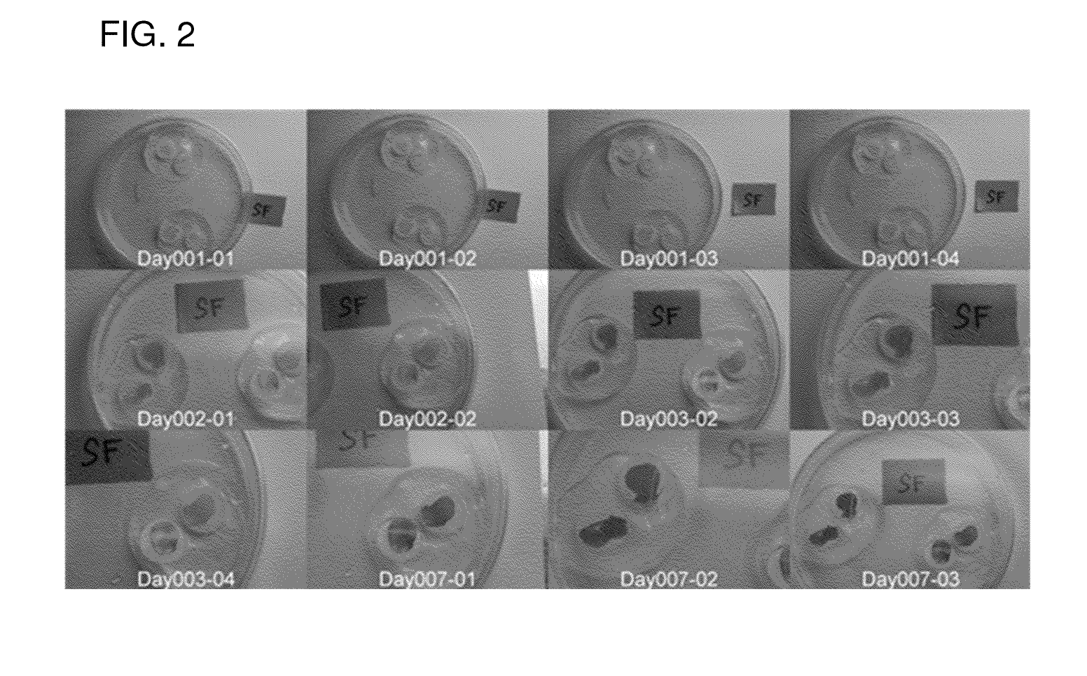

Algae (Nanochloropsis salina 1776) were successfully introduced into the hydrogel during the gelation process (cross-linking) and cultivated inside or on the surface of the hydrogel foam beads. Algae growth was monitored for one weak and the algae demonstrated growth and biomass spreading throughout the hydrogel (see FIG. 2). Further, CO₂ losses were negligible and the evaporation rate was reduced by 20-30% compared to an uncovered film.

### Example 2

### Synthesis, Preparation and Properties Light Frequency-Shifting Agents

This example provides methods for the synthesis and preparation of light frequency-shifting agents, more specifically NQDs. Further provided are measurements and analyses of their properties.

### Synthesis:

Materials and instrumentation: Cadmium oxide, oleic acid. (90%), 1-octadecene (ODE, 90%), dioctylamine (95%), octadecylamine, zinc oxide, sulfur, selenium pellet, and trioctyl phosphine (TOP) were purchased from Sigma-Aldrich® and used without further purification. Trioctyl phosphine oxide (TOPO) (90%) was purchased from Strem Chemicals, Inc. (Newburyport, Mass.) and used without further purification. Absorption and emission spectra were recorded on a CARY™ UV-VIS-NIR spectrophotometer and a NanoLog™ fluorometer, respectively. TEM images were taken on a JEOL™ 2010 transmission electron microscope.

Synthesis of CdSe-based “NQDs.” The synthesis of giant CdSe/thick-shell NQDs was based on a SILAR approach with minor modification, as described in Xie et al., “Synthesis and Characterization of Highly Luminescent CdSe-Core CdS/Zn_{0.5}Cd_{0.5}S/ZnS Multishell Nanocrystals,” J. Am. Chem. Soc. (2005), v. 127, pp. 7480-7488. The CdSe core was prepared by injection of 1 ml 1.5 M Se-TOP solution into a hot solution containing 1.5 g octadecylamine, 0.5 g TOPO, 5 g octadecene, and 0.2 mmol Cd-oleate under standard air-free conditions. After injection of Se-TOP at 290° C., the temperature was set at 250° C. for CdSe NQD growth. After ten minutes, the solution was cooled down to room temperature, and CdSe NQDs were collected by precipitation with acetone and centrifugation. CdSe core NQDs were re-dispersed in hexane.

About 1.5×10⁻⁷ mol CdSe core NQDs in hexane were put into a 100 ml flask with 3 g ODE and 3 g dioctylamine. Instead of the primary amine used in the literature procedures, a secondary amine was chosen as the ligand to prevent the reaction between Cd-oleate and the amine ligands. 0.2 M elemental sulfur dissolved in ODE, 0.2 M Cd-oleate in ODE and 0.2 M ZnₓCd₁₋ₓ-oleate (x=0.13, 0.49, 0.78, respectively) were used as precursors for shell growth. The quantity of precursors for each monolayer of shell was calculated according to the volume increment of each monolayer shell, considering the changing total NQD size with each successive monolayer grown. The reaction temperature was set at 240° C. Growth times were 1 hour for sulfur and 3 hours for the cation precursors. CdSe-based NQDs of different shell compositions were synthesized. For example, CdSe cores with 19 monolayers of CdS shell, CdSe cores with 11 monolayers of CdS shell plus 6 monolayers of ZnₓCd₁₋ₓS alloy shell and 2 monolayers of ZnS shell (19 monolayers of shell total), and CdSe cores with 10 monolayers of CdS shell plus 8 monolayers of ZnS shell (18 monolayers of shell total) were prepared. Other thinner shells, such as CdSe cores with 2 monolayers of CdS and 2 monolayers of ZnS shell, and CdSe cores with 2 monolayers CdS shell, 3 monolayers of ZnₓCd₁₋ₓS alloy shell and 2 monolayers of ZnS shell, were also prepared for control studies.

To check the stability of NQDs with regard to purification, the NQDs of this Example were precipitated from growth solution and dispersed in hexane as described above. Further, they were subsequently subjected to multiple “purification” steps in which they were substantially completely precipitated with methanol followed by re-dispersion in hexane. This process was repeated up to seven times and without loss of solubility. Quantum yields in emission were measured in growth solution, as well as after each precipitation/re-dispersion cycle. As controls, CdSe core NQDs and standard CdSe core/shell and core/multi-shell NQDs were similarly prepared, purified and measured for quantum yield.

### Water-Soluble NQDs

CdSe NQDs were transferred into water by stirring purified nanocrystals (˜5×10⁻⁹ mol) in hexane with 1 mmol mercaptosuccinic acid in 5 ml deionized water overnight. Mercaptosuccinic acid was neutralized by tetramethylammonium hydroxide in water. The pH of the water was about 7. Mercaptosuccinic acid-capped NQDs were collected by centrifugation, and were then re-dispersed in a small amount of water and precipitated again using an excess of methanol to remove excess mercaptosuccinic acid. Finally, the purified mercatosuccinic acid-capped NQDs were dispersed in deionized water to form optically clear solutions.

### Properties:

Quantum yields (QYs) for the NQDs and the various NQD control samples in hexane were measured by comparing the NQD emission with that of an organic dye (Rhodamine 590 in methanol). The excitation wavelength was 505 nm and emission was recorded from 520 nm −750 nm. The QY of Rhodamine 590 was taken to be 95%, and those for the NQD samples were calculated by comparing the emission peak areas of the NQDs with the known dye solution. Specifically, the NQD QYs were calculated using the formula:

QY_{NCs}=Abs_{dye}/Abs_{NCs}*Peak area of NQDs/peak area of dye*QY_{dye}*(RI_{dye}²/RI_{NCs}²)

RI_{dye}—refractive index of dye solution in methanol, =1.3284

RI_{NCs}—refractive index of CdSe nanocrystals solution in hexane, =1.3749

Typically, the absorbance of the dye and the CdSe NQD solutions were controlled from about 0.01 and to about 0.05 optical density. The absorbance and emission for each sample were measured twice at two different concentrations. The reported NQD QYs comprise averages of the two measurements. In an effort to obtain more accurate results, at least five measurements were conducted at different concentrations.

Hydrodynamic (total) diameters (HD's) were measured via Dynamic Light Scattering (DLS). DLS measurements were performed in 1 cm quartz cuvettes in a 90° angle configuration with a 633 nm laser source using a Palo Alto Nano-Zeta Sizer from Malvern™ Instruments. All DLS measurements are 12 run averages and were carried out at 20° C. after a 2 min. equilibration period. Viscosity and refractive index values for the solvent (toluene or hexane), and the refractive index for the semiconductor material were taken from the CRC Handbook of Chemistry and Physics, 81^{st} Ed. (2000-2001). The refractive index for all NQDs was assumed to be similar to that reported for CdS. Unimodal HD distributions were obtained for all NQDs at different concentrations. HD values were corrected against polystyrene (PS) latex bead standards (Duke Scientific) in water in the 20-60 nm region (Table 1).

TABLE 1
Dynamic Light Scattering Analysis of NQDs

| Sample | HD (nm)^{a} | PDI (%) | PDI (nm) |
|---|---|---|---|
| CdSe/11CdS/6Cdₓ0Zn_{y}S/2ZnS | 25.1 | 9.5 | 2.4 |
| CdSe/11CdS/6CdₓZn_{y}S/2ZnS | 24.5^{b} | 9 | 2.2 |
| CdSe/19CdS | 23 | 3.2 | 0.7 |

^{a}FID = Hydrodynamic (total) diameter; measured in toluene or hexane after one or two precipitations with MeOH. HD values are corrected against polystyrene latex standards (20-60 nm).
^{b}Unwashed: measured in growth solution.

No evidence for aggregation or clustering of any of the NQD samples in solution was observed via DLS. Specifically, control experiments with mixtures of PS standards showed that DLS predominantly “sees” larger particles or aggregates: For example, a 5:5 mixture of 20 nm and 50 nm PS standards, respectively, gave an average or “effective” HD of 50.2 nm; whereas a 9:1 mixture of 20 nm and 50 nm PS standards, respectively, gave an average or “effective” HD of 39.5 nm. Thus, DLS is particularly sensitive in identifying even relatively small fractions of larger aggregates or clusters.

Further, the DLS results compare well to size analysis by TEM. According to TEM, the NQD CdSe/19CdS is 15.5+/−3.1 nm in size, and the NQD CdSe/11CdS/6Cd.sub.xZn.sub.yS/2ZnS is 18.3+/−2.9 nm in size. After adding two ligand layers (about 3 nm considering presence of TOP and even longer oleylamine, etc.), the total diameters then range between 15.4 nm-21.6 nm for CdSe/19CdS and 18.4 nm-24.2 nm for CdSe/11CdS/6CdₓZn_{y}S/2ZnS. These results are consistent with that obtained by DLS (Table 2), and especially so considering that DLS sizes are inherently skewed towards the larger side of a size distribution (see above control study using mixtures of differently sized PS bead standards).

TABLE 2
Comparison of TEM-derived total size with DLS-derived total size

| | TEM total size | | DLS total size (HD) | |
|---|---|---|---|---|
| | (from histograms + | | (from corrected HD′s in | |
| Sample | 2 ligand layers) | | Table 1 above) | |
| Giant alloy | 15.4-21.6 | mm | 22.7-27.5 | nm (washed) |
| | | | 22.3-26.7 | nm (unwashed) |
| Giant 19CdS | 18.4-24.2 | mm | 22.3-23.7 | nm |

Sample preparation: NQDs and control NQD samples (controls: Qdot® 655 ITK (Invitrogen™), CdSe/2CdS/2CdZnS/nZnS (n=2 or 3), and CdSe cores) were diluted in HPLC grade toluene or deionized water (in the case of carboxylate-thiol-exchanged dots) to a concentration of about 0.1-50 pM range. Thin films of these highly dilute solutions were made on new 0.5 mm thick quartz slides (pre-cleaned with chloroform, acetone, methanol and air-pressure). Single-dot imaging: NQDs were excited by focusing a 532 nm CW laser (˜100 mW) onto a spot of about 50 .mu.m in diameter. PL of individual NQDs was collected through a 40×, 0.6NA microscope objective and imaged onto a liquid-nitrogen-cooled CCD detector. The images covered an area about 40×45 μm² in size and were acquired using a 100 ms integration-time. Series of up to 18,000 such images were acquired consecutively. Each image in the series was separated by about 100 ms of CCD read-out time. A computer program designed to extract the intensity fluctuations of all individual NQDs for these series of images was used to analyze blinking statistics. The distribution of on-time fractions (total on-time/total experiment time) displayed in the main text were extracted from about 500 NQDs. It is important to note that the PL intensity of individual NQDs varies widely. When the PL of a relatively weakly emitting NQD goes below a designated threshold, the program automatically counts this event as an “off” state. Therefore, this analysis program inherently underestimates the total % on-time. Photobleaching analysis: To quantitatively analyze the photostability of our NQDs in comparison to the control samples, the total number was monitored of different NQDs observed in every 18 s time interval (extracted from 100 images taken consecutively) for 1080 s (corresponding to 6000 images).

### Example 3

### Preparation of Biocompatible Polymer Having a Light Frequency-Shifting Agent

This example provides methods for making a biocompatible polymer having light frequency-shifting agents (e.g., quantum dots as described herein).

Water-soluable NQD's (see Example 2 above) will be added to the aqueous alginic solutions described in the protocols in Example 1. Either the Direct Capture or Carbonate Decomposition method may be used. The NQD's will be added to the aqueous alginic solution at anywhere from 1-10 μM (or 1, 2, 3, 4, 5, 6, 7, 8, 9 or 10 μM) concentration. The solution will be mixed for approximately 30 minutes to ensure a homogenous solution. Inorganic carbon will be added to the solution, which will then be cross-linked as described in Example 1. The concentration of NQD's in the hydrogel will be (by % wt) about from 0.05% to about 1% (or 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1%).

The NQD's in the hydrogel will exhibit the same or similar optical properties as the NQD's in the absence of hydrogel.

### Example 4

### Transgenic Algae Having Improved Photosynthetic Light Energy Utilization

This example provides methods for improving photosynthetic light energy use in algae.

Transgenic C. reinhardtii strains having a range of LHCII antenna sizes that were intermediate between WT and a Chl b less strain which entirely lacks LHCII were generated. Transgenic algae having intermediate LHCII content are capable of state transitions as well as non-photochemical quenching of excess energy via the violaxanthin-zeaxanthin cycle. Algae with intermediate antennae sizes also have substantially higher growth rates than WT or Chl b lacking algal strains when grown autotrophically at saturating (in WT) light intensities while having growth rates similar to WT at low light intensities.

Generating Transgenic Algae for RNAi-Mediating Silencing of CAO

The plasmid for inducing RNAi-mediated silencing of the chlorophyllide a oxygenase (CAO) gene in C. reinhardtii strain CC-424 (arg2 cw15 sr-u-2-60 mt-, Chlamydomonas Genetic Center) was constructed using a genomic-sense/cDNA-antisense strategy. The first two exons and introns of the CAO gene were amplified by PCR using GCTTTCGTCATATGCTTCCTGCGTCGCTTC (SEQ ID NO: 27) and CTCTGGATCCGTCTGTGTAAATGTGATGAAGC (SEQ ID NO: 28) as forward and reverse primers respectively and the resulting product was digested with restriction enzymes NdeI and BamHI. The corresponding cDNA region spanning exons 1 and 2 of the CAO gene was amplified using GACGAATTCGTCAGATGCTTCCTGCGTCG (SEQ ID NO: 29) and CTCTAGATCTGTCGCCTCCGCCTTCAGCTC (SEQ ID NO: 30) as the forward and reverse primers and digested with restriction enzymes EcoRI and BglII. The genomic DNA and cDNA fragments were cloned into the PSL18 vector using the NdeI and EcoRI sites to generate the CAO-RNAi vector (FIG. S1A). The psaD promoter and terminator cassette of the PSL18 vector was used to drive RNAi. The PSL18 vector contains the paromomycin resistance gene driven by the Hsp70/RbcS2 fusion promoter, placed in tandem with the PsaD promoter and terminator cassette. Chlamydomonas transformants generated using the CAO-RNAi vector were selected based on resistance to paromomycin

For the generation of the CAO-RNAi lines (CR), the cell wall-less CC-424 C. reinhardtii strain was transformed with 1 μg of ScaI linearized CAO-RNAi plasmid by glass bead-mediated nuclear transformation. Transformants were selected on TAP agar plates containing 100 μg/mL of 1-arginine and 50 μg/mL of paromomycin. Transformants were further screened by pigment extraction and spectrophotometric analysis of Chl alb ratios, which were expected to increase as a consequence of CAO gene silencing. For this, cells were grown in culture tubes containing 3 mL of high salt (HS) media+arginine (100 μg/mL) for 5-6 days under continuous illumination of ˜50 μmol light m⁻² s⁻¹ and the relative amounts of Chl a and b were determined as described in Arnon. The presence of the CAO-RNAi and paramomycin resistance cassettes in the transgenics was further confirmed by PCR using a forward primer binding within the PsaD promoter (GTATCAATATTGTTGCGTTCGGGCAC) (SEQ ID NO: 31) and a reverse primer binding within the CAO-RNAi cassette (ATCAGTTGCGTGCGCCTTGCCAAACC) (SEQ ID NO: 52) to yield an ˜780 bp fragment as well as a forward primer binding within the Hsp70/Rbcs2 fusion promoter (GGAGCGCAGCCAAACCAGGATGATG) (SEQ ID NO: 32) and a reverse primer (GTCCCCACCACCCTCCACAACACG) (SEQ ID NO: 33) binding within the paramomycin resistance gene to yield a 630 bp fragment.

Methods Used to Confirm Presence of Transgenics and Phenotypic Traits

RNA was isolated from 25 mL of the log phase cultures grown under 50 μmol m⁻² s⁻¹ light using Trizol according to the manufacturer's instructions (TRI Reagent®, Ambion, Catalog #AM9738). DNase (Promega, Catalog #M610A) treated RNA samples (2 μg) were reverse transcribed using the qScript™ cDNA SuperMix kit (Quanta Biosciences). Real-time quantitative RT-PCR was carried out using an ABI-Step one plus using the SYBR Green PCR Master Mix Reagent Kit (Quanta Biosciences). The Chlamydomonas CBLP gene was used as internal control and was amplified in parallel for gene expression normalization. The forward and reverse primers used for amplification of the CBLP gene were GCAAGTACACCATTGGCGAGC (SEQ ID NO: 34) and CCTTTGCACAGCGCACAC (SEQ ID NO: 35) respectively and the forward and reverse primers used for the amplification of the CAO gene were GACTTCCTGCCCTGGATGC (SEQ ID NO: 36) and GGGTTGGACCAGTTGCTGC (SEQ ID NO: 37) respectively. The PCR cycling conditions included an initial polymerase activation step at 95° C. for 10 min, followed by 40 PCR cycles at 95° C. for 15 s, 61° C. for 15 s and 72° C. for 30 s and a final melting step of 60-95° C. each for 15 s. The quantification of the relative transcript levels was performed using the comparative CT (threshold cycle) method.

For Chl fluorescence induction analysis, cell suspensions of the parental WT and transgenic Chlamydomonas were adjusted to a Chl concentration of ˜2.5 μg Chl/mL. Flash Chl fluorescence induction was measured using the FL-3500 fluorometer (Photon System Instruments) as described in Nedbal et al. The cells were dark adapted for 10 min prior to the experiment. Chl fluorescence was induced using non-saturating continuous illumination and Chl fluorescence levels were measured every 1 μs using a weak pulse-modulated measuring flash. The values of Chl fluorescence were normalized to the maximum achieved for a given sample. For the state transition experiments, low light grown cultures were dark adapted or pre-illuminated with 715 nm or 650 nm light for 10 min prior to the induction of Chl fluorescence. The actinic flash duration for this experiment was set to 50 μs and Chl fluorescence was measured every 1 μs.

The CC-424, CR-118 and CR-133 strains, and the Chlamydomonas Chl b less mutant, cbs3, were grown in high salt (HS) under low light intensities (50 μmol light m⁻² s⁻¹) with continuous shaking at 225 rpm for 6 days. Cells were harvested by centrifugation at 3000×g for 5 min at 4° C. The cell pellet was resuspended in buffer A (0.3 M sucrose, 25 mM HEPES, pH 7.5, 1 mM MgCl₂) plus 20 μL/mL of protease inhibitor cocktail (Roche), to yield a final Chl concentration of 1 mg/mL. Cells were then broken by sonication (Biologics, Inc., Model 300 V/T Ultrasonic Homogenizer) two times for 10 s each time (pulse mode, 50% duty cycle, output power 5) on ice. The unbroken cells were pelleted by centrifugation at 3000×g for 2 min at 4° C. The supernatant was centrifuged at 12,000×g for 20 min and the resulting pellet was washed with buffer A. The sample was subjected to a second centrifugation step at 11,000×g to collect thylakoids. Thylakoid membranes were then solubilized with LiDodSO₄. Briefly, 15 μg Chl equivalent of thylakoids was solubilized in a buffer containing 50 mM Na₂CO₃, 50 mM dithiothreitol, 12% sucrose and 2% lithium dodecyl sulfate to yield a final Chl concentration of 1 mg/mL and a Chl/LiDodSO₄ (wt/wt) ratio of 1:20. The sample was gently shaken for 60 s. Equal amounts of the sample buffer (62.5 mM Tris-HCl, pH 6.8 and 25% glycerol) were added to the solubilized thylakoids before loading. The samples were then loaded onto a Ready Tris-HCl Gel (Bio-rad 161-1225) and LiDodSO₄ and EDTA were added to the upper reservoir buffer (25 mM Tris, 192 mM glycine) to a final concentration of 0.1% and 1 mM, respectively. Electrophoresis was performed at 4° C. in the dark for 2-2.5 h at 12 mA constant current.

The oxygen evolving activity of the log-phase cultures (0.4-0.7 OD_{750 nm}) of CC-424, CR-118, CR-133, CC-2677 (cw15 nit1-305 mt-5D, Chlamydomonas Genetic Center) and cbs3 was assayed using a Clark-type oxygen electrode (Hansatech Instruments) using low light (50 μmol photons m⁻² s⁻¹) grown cultures. Cells were resuspended in 20 mM HEPES buffer (pH 7.4) and the rate of oxygen evolution was measured as a function of increasing light intensity (650 nm wavelength red light). The photon flux density was maintained for 1.5 min at 50, 150, 300, 450, 600, 750 and 850 μE m⁻² s⁻¹ of red light to obtain a light saturation curve of photosynthesis. The same experiment was repeated in the presence of 10 mM NaHCO₃. Light saturation curves were normalized on the basis of Chl as well as cell density (OD 750 nm).

Photoautotrophic growth of the CC-424, CR-118, CR-133, CC-2677 and cbs3 Chlamydomonas strains was measured in a time dependent manner, in 125 mL flasks in liquid HS media, at either low light (LL, 50 μmol photons m⁻² s⁻¹) or high light (HL, 500 μmol photons m⁻² s⁻¹) conditions with constant shaking at 175 rpm. The media was supplemented with 100 μg/mL of 1-arginine. The optical density of the cultures was monitored on a daily basis at 750 nm using a Cary 300 Bio UV-vis spectrophotometer.

Chlamydomonas cultures were grown in low (50 μmol photons m⁻² s⁻¹) and high light (500 μmol photons m⁻² s⁻¹) intensities for 5 days. Cells were centrifuged at 3000 rpm for 3 min and immediately frozen in liquid nitrogen and lyophilized. Carotenoids and Chls were extracted with 100% acetone in the dark for 20 min. After incubation samples were centrifuged at 14,000 rpm for 2 min in a microfuge and the supernatant was transferred to a glass tube and dried under vacuum. The dried samples were resuspended in 1 mL of acetonitrile:water:triethylamine (900:99:1, v/v/v) for HPLC analysis. Pigment separation and chromatographic analysis were performed on a Beckman HPLC equipped with a UV-vis detector, using a C18 reverse phase column at a flow rate of 1.5 mL/min. Mobile phases were (A) acetonitrile/H₂O/triethylamine (900:99:1, v/v/v) and (B) ethyl acetate. Pigment detection was carried out at 445 nm with reference at 550 nm. Pigment standards were bought from DHI, Denmark.

### Results

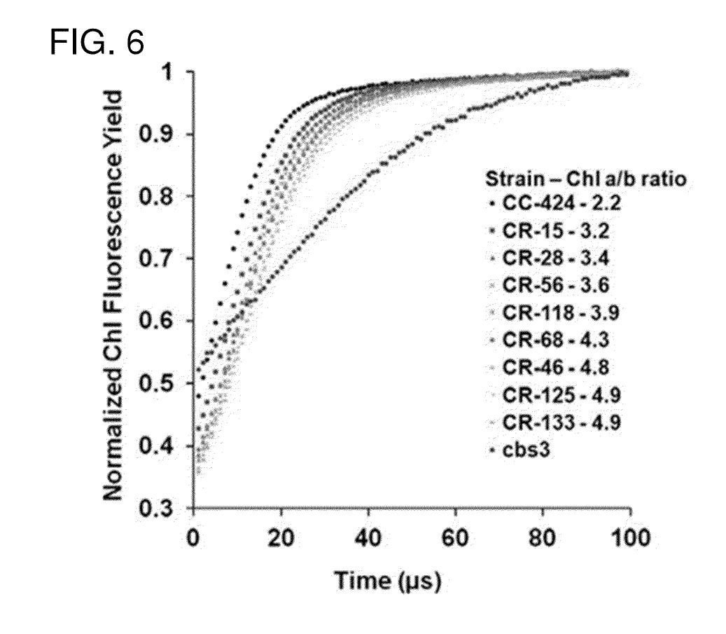

To generate transgenic algae with reduced Chl b levels and intermediate PSII antenna size, an RNAi approach was used to modulate the expression of CAO, the gene responsible for the synthesis of Chl b via the oxidation of Chl a. A genomic-sense/cDNA-antisense construct spanning the first two exons of the CAO gene was used to generate the CAO-RNAi transgene. After transformation with the CAO-RNAi plasmid, transgenics were selected on the basis of paromomycin resistance encoded on the integrating plasmid. Eight independent CAO-RNAi (CR) transgenics with Chl alb ratios ranging from 3.2 to 4.9 were generated and confirmed by PCR for the presence of the RNAi cassette as well as the paromomycin resistance marker (FIG. 6). To determine the effects of reduced Chl b levels on the PSII antenna absorption cross-section, we measured Chl fluorescence induction kinetics in the CR strains and their parent (CC-424) as well as a Chl b less mutant, cbs3. The rate at which Chl fluorescence rises is indicative of the rate of closure of PSII RCs and the PSII antenna size under conditions of non-saturating, continuous illumination. As shown in FIG. 6, the CR transgenics had slower Chl fluorescence induction kinetics relative to WT (Chl alb=2.2) reflective of a smaller PSII antenna size and only reached ˜75 to 85% PSII RC normalized maximum fluorescence level when the parent strain had reached 90% of saturation. Significantly, the PSII RC closure rate was inversely correlated with the Chl alb ratio, implying that the Chl alb ratio is a direct indicator of the antenna size over the Chl alb ranges tested. Reductions in LHCII content in the two CR strains and the cbs3 mutant were also confirmed using non-denaturing polyacrylamide gel electrophoresis. The two CR transgenics (CR-118 and CR-133), having Chl alb ratios representative of an intermediate and the highest CR Chl alb ratio, had a ˜20-30% reduction in LHCII (CPII band) content relative to WT. The CPII band was absent in the cbs3 mutant. As expected, reductions in CR LHCII content were associated with reductions in CAO mRNA levels. It is noteworthy that large reductions in CAO transcript levels in the CR transgenics relative to their parental WT led to only modest decreases (30-48%) in Chl b levels. It has previously been shown that low levels of CAO protein are sufficient to support normal levels of Chl b synthesis. Therefore, it is likely that low CAO transcript levels in the CR lines are sufficient to support moderate levels of Chl b synthesis. Interestingly, chlorophyll pigment analyses of the CR strains grown under low and high light conditions showed some plasticity in Chl b levels as a function of growth light intensity. In contrast to the parental WT, Chl alb ratios were significantly higher (p<0.01) in high-light grown cultures of the CR strains than in low-light grown cultures. The CR lines also exhibited substantial decreases in Chl b (41-43%) content and antenna size when grown in high relative to low light intensities. In addition, we observed a 40-60% decrease in the total Chl content per unit dry weight in high light grown cultures of strains compared to low light grown cultures.

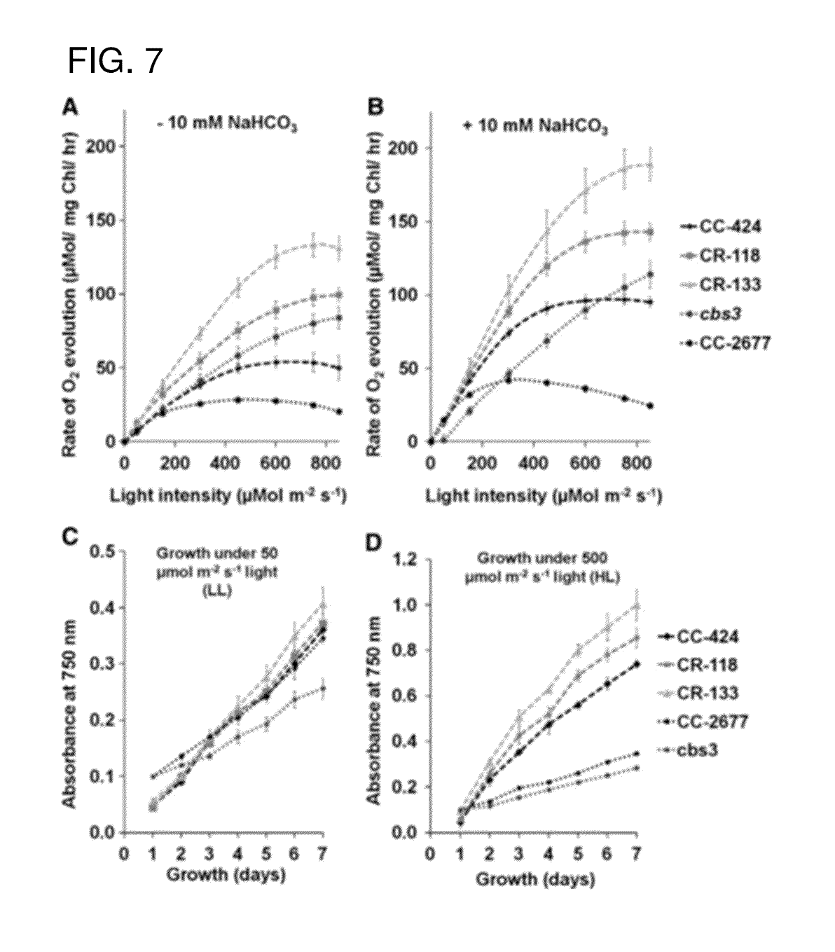

To study the effect of reduced LHCII abundance on light-dependent rates of photosynthetic oxygen evolution, we compared rates of photosynthesis in the two CR strains, the cbs3 mutant, and their parent strains, CC-424 and CC-2677, respectively. The CR lines had from about 2 to 2.6 fold higher light-saturated photosynthetic rates (Pₘₐₓ) than WT on a Chl basis (FIG. 7A) and up to ˜1.5-2 fold greater photosynthetic rates when measured in the presence of saturating inorganic carbon levels (10 mM NaHCO₃) (FIG. 7B). The higher photosynthetic rates in the presence of saturating levels of bicarbonate are presumably associated with the active transport of bicarbonate into the cells resulting in the elevation of internal CO₂ concentrations. Similar increases in Pₘₐₓ were also observed in the CR transgenics when oxygen evolution rates were expressed on the basis of cell density indicating that the reduction in Chl content per cell did not substantially bias the rates of photosynthesis reported on a Chl basis for the CR transgenics. In contrast, we observed a ˜4 fold increase in Pₘₐₓ for the Chl b less mutant, compared to its parent measured on a Chl basis, but when expressed on a cell density basis, there was only a 2-fold increase in light-saturated rates of photosynthesis relative to WT indicative of substantial reductions in total Chl/cell.

To determine the impact of antenna size on photoautotrophic growth, growth rates under limiting and saturating light conditions (50 and 500 μmol light m⁻² s⁻¹) were measured. Growth of the CR transgenics was unimpaired compared to its parental WT under limiting light intensities (FIG. 7C). On the other hand, the cbs3 mutant had a 25% reduction in stationary phase cell density under low light growth conditions relative to its parent WT strain (CC-2677), presumably due to the smaller optical cross section of the antennae. Under saturating light intensities, however, the CR strains had ˜15 to 35% higher stationary phase culture densities than the parental WT, while the cbs3 strain had a substantially reduced stationary phase cell density (˜80% of WT) indicating that photosynthetic and growth rates were not correlated in this mutant presumably reflecting additional impairments in photosynthetic activities (FIG. 7D).

In C. reinhardtii, the peripheral PSII antenna is able to migrate laterally between PSII and PSI, in a process known as state transitions, to balance the excitation energy distribution between the two photosystems and to regulate the ratio of linear and cyclic electron flows. Linear electron transfer produces ATP and NADPH, while cyclic electron transfer driven by PSI produces only ATP. Increasing the antenna size of the PSI complex facilitates cyclic electron transfer and has been shown to enhance ATP production and support the optimal growth of Chlamydomonas. Thus, LHCII minus strains would presumably have an impaired ability to synthesize ATP by cyclic photophosphorylation. To assess the impact of reduced LHCII content on the ability to carry out state transitions, Chl fluorescence induction kinetics were measured in low-light grown WT, cbs3 and CR cells that were either dark adapted, pre-illuminated with PSI (715 nm), or pre-illuminated with PSII (650 nm) light. PSI light pre-illumination promotes LHCII migration from PSI to PSII while PSII light does the opposite. An increase in the PSII antenna size would accelerate Chl fluorescence rise kinetics and increase the maximal Chl fluorescence level at sub-saturating light intensities. As expected, CR and WT strains had faster Chl fluorescence rise kinetics and achieved greater maximum Chl fluorescence levels following pre-illumination with PSI light. However, no observable increase in Chl fluorescence yield was observed in the cbs3 strain following pre-illumination with PSI light, indicating that cbs3 lacked the ability to carry out state transitions. The absence of LHCII and state transitions and presumably diminished potential for cyclic photophosphorylation and ATP synthesis, may partially account for the impaired photoautotrophic growth of cbs3.

The peripheral PSII antenna binds an array of carotenoids involved in energy capture or dissipation. Under high light intensities acidification of the chloroplast lumen activates de-epoxidases that convert violaxanthin into zeaxanthin. Violaxanthin transfers energy to Chl facilitating light harvesting at low light intensities while zeaxanthin dissipates excess Chl excited states at high light as heat. To examine the effects of reduced antenna size on carotenoid levels, we carried out pigment analyses of low and high light grown strains. As expected, we observed a decrease in carotenoid levels in low-light grown CR (76-80% of WT) and cbs3 (76% of WT) strains. The high-light grown CR parental WT strains had a 2.8 and 3 fold increase in antheraxanthin and zeaxanthin pools respectively, compared to low-light grown cells. However, high-light grown CR lines displayed a 15-30% increase in de-epoxidation status (antheraxanthin+zeaxanthin/violaxanthin+antheraxanthin+zeaxanthin) compared to their WT parental strain. Hence, even greater increases in the levels of antheraxanthin and zeaxanthin were observed in high-light grown CR-118 (5 and 5.6 folds) and CR-133 (5.3 and 6.8 folds) than in its parental (CC-424) WT (3 folds), which is indicative of a more active xanthophyll cycle in the CR transgenics. Further, a 1.2 fold increase in lutein content was observed in high-light grown CR-133 relative to low-light grown cells. In contrast, the cbs3 parent strain (CC-2677) had no change in its carotenoid de-epoxidation state or xanthophyll cycle carotenoid levels under high-light relative to low-light growth. However, the CC-2677 strain had higher beta-carotene (2 folds) levels when grown under high versus low-light growth conditions (FIG. S4), suggesting that this strain differs in its carotenoid regulation from the WT parent (CC-424) of the CR transgenics. Unexpectedly, high-light grown cbs3 exhibited a 1.8 fold increase in its carotenoid de-epoxidation state compared to its parent (CC-2677) and had a 2-fold increase in zeaxanthin content, however, the total levels of de-epoxidated carotenoids were substantially lower in CC-2677 derived lines than in CC-424 derived lines. Similar to its parent strain, an elevation (2-fold) in beta-carotene levels was also observed in high-light grown cbs3 relative to low-light growth. Overall, the differences in carotenoid de-epoxidation levels observed in the truncated antenna mutants and WT parental strains indicate that xanthophyll cycle activity is not directly correlated with LHCII content in these particular Chlamydomonas strains.

### Conclusion

There is an inverse relationship between Chl alb ratios and the PSII antenna size. CR transgenics with intermediate antenna sizes grew at WT rates at low light intensities but had ˜15 to 35% higher culture densities than their parental WT strain when grown at saturating light intensities (25% of full sunlight intensity). These studies indicate that at low light intensities the size of the peripheral antennae complex is more than sufficient to support the maximal rates of photosynthesis and that the reductions in antennae size within the range tested had no impact on algal growth rates. The large antenna absorption cross-section of wild type algae reduces available light for competing algal species providing a selective advantage even at very low light levels.

Truncation of the peripheral LHCII light harvesting complex in green algae leads to increased photosynthetic energy conversion efficiency by reducing flux constraints between light capture and linear electron flow at high light intensities. However, unlike algae that lack the PSII peripheral antenna, the CR transgenics retain the photoprotective functions of the antenna and to quench excess potentially damaging Chl excited states and combine improved photon capture and energy conversion with the ability to dynamically regulate light distribution between the photosystems to support cyclic photophosphorylation.

While the above example provides an RNAi based method for improving photosynthetic energy conversion in algae, other methods are available to one of ordinary skill in the art to modulate light harvesting antenna size.

### Generating Transgenic Algae Having the NAB1 Regulated CAO Gene Construct

For the construction of the NAB 1 regulated CAO gene, the CAO gene was amplified with:

N1BSCAO-F
forward primer;
5′-ATCTTCATATGGGCCAGACCCCCGCAGGGCTTCCTGCGTCGCTTC
AACGCAAGG-3′; SEQ ID NO: 38)
and
CAO-Rev
reverse primers
5′-TAGAATCTAGACrAGTTGTCCATGTCATCCTCGTCCA-3′;
SEQ ID NO: 39)

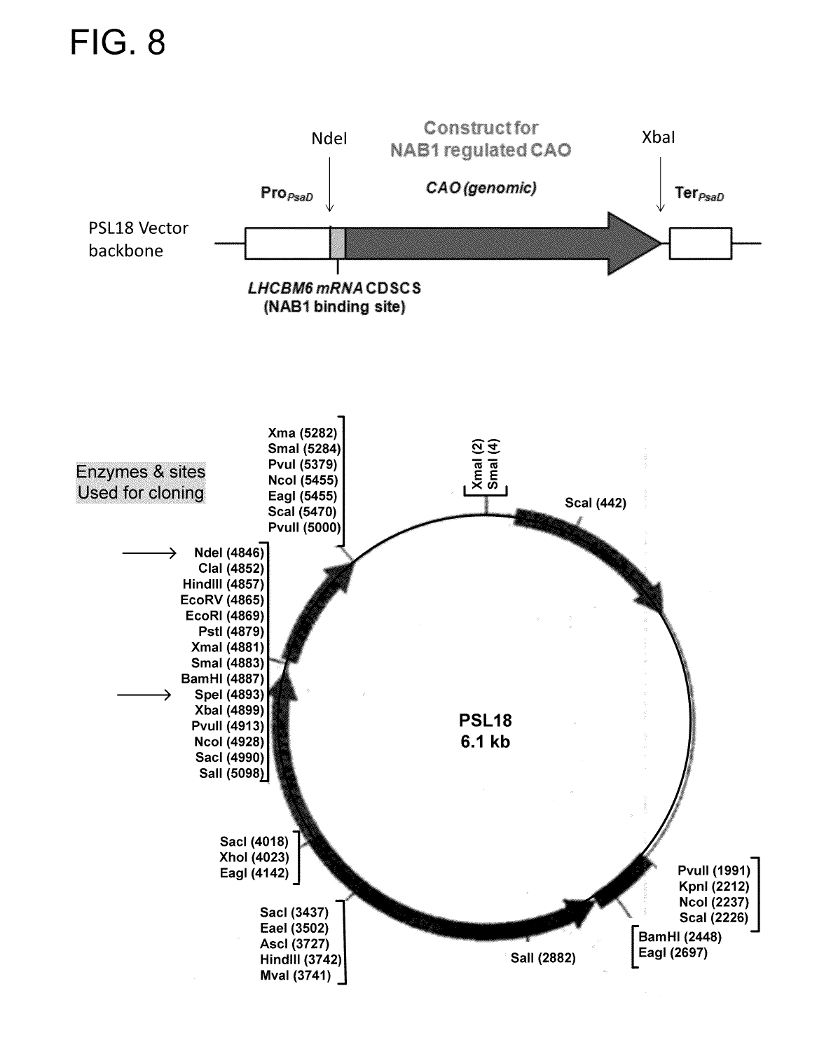

using genomic DNA isolated from Chlamydomonas strain CC-2137 (Chlamydomonas Genetic Center) as template. The 13-bp NAB 1 binding site (NI BS) in this construct corresponds to the sequence 5′-GCCAGACCCCCGC-3′ (SEQ ID NO: 40). Genomic DNA was extracted from Chlamydomonas using the xantine buffer protocol (described in Tillett and Neilan, (2000); J. Phycol. 36: 251-258). The Nde1 and Xba1 restriction sites were used in cloning of the amplified gene into the nuclear gene expression vector PSL18, to generate the PSL18-N1 BS-CAO vector, which is shown schematically in FIG. 8).

### Forward and Reverse Primers:

CAOEx12GS_F:
(SEQ ID NO: 41)
5′-GCTTTCGTCATATGCTTCTGCGTCGCTTC-3′
CAOEx12GS_R:
(SEQ ID NO: 42)
5′-CTCTGGATCCGTCTGTGTAAATGTGATGAAGC-3′
CAOEx12CAS_F:
(SEQ ID NO: 43)
5′-GACGAATTCGTCAGATGCTTCCTGCGTCG-3′
CAOEx12CAS-R:
(SEQ ID NO: 44)
5′-CTCTAGATCTGTCGCCTCCGCCTTCAGCTC-3′
PSLI18-F-seq:
(SEQ ID NO: 45)
5′-CAGTCCTGTAGCTTCATACAAACATA-3′
PSLi1-R-seq:
(SEQ ID NO: 46)
5′-GATCCTCCTGTGGCTAATTGACC-3′

To generate control plasmids in which the CAO gene was not preceded by the NAB 1 binding site (PSL18-CAO), or had an altered NAB 1 binding site (PSL18-altN1BS-CAO), the CAO-F (5′-ATCTTCATATGCTTCCTGCGTCGCTTCAAC-3′ SEQ ID NO: 47) or altN1BSCAO-F (5′-ATCTTCATArGGGGCAAACACCGGCGGGCCTTCCTGC-3′; SEQ ID NO: 48) forward primers were used respectively, in combination with the same reverse primer as above. All the resulting plasmids PSL18-CAO, PSL18-N1 BS-CAO and PSL18-altN 1BS-CAO were sequenced using the PSL18-psaD-F (5′-GTTAGGTGTTTGCGCTCTTGAC-3′; SEQ ID NO: 49) and CAO-seq primers (5′-GGCGAGTGAGCATATTCGTCC-3′; SEQ ID NO: 50).

In order to demonstrate that the NAB 1 binding domain interacted with the NAB 1 protein we generated NAB 1 binding domain mutants and assessed their ability to undergo light-dependent changes in chlorophyll b content. A gene construct in which the 13-bp LHCBM6 mRNA CDSCS or NAB 1 binding site (abbreviated here to NIBS) (SEQ ID NO: 40) was placed at the 5′ end of the CAO gene, was introduced into the CAO knock out stain cbs-3 by particle gun bombardment mediated transformation to generate the N1 BS-CAO transgenic cell lines. As a control, the CAO knock out strain was complemented with the wild-type CAO gene lacking the 5′ NIBS sequence to generate the complemented wild-type. A mutagenized NAB 1 binding site (different from LHCBM6 mRNA CDSCS by 4 bp) (5′-GGCAAACACCGGC-3 ‘; SEQ ID NO: 51) was also constructed and inserted into the 5’ coding sequence of the CAO gene and transformed into the Chi b-less strain, cbs-3, to generate the altN1BS-CAO transgenic cell lines.

In all cases, the PsaD promoter was used to drive the expression of the gene construct so as to decouple any potential effects of the native CAO promoter. The resulting transgenic clones were selected initially on the basis of antibiotic resistance and then further screened by pigment extraction and quantification. Selected transgenic clones having Chi a/b ratios intermediate between wild-type (CC-2137) and Chi b-less cells were confirmed for the presence of the transgene by PCR (data not shown). The amplified region was verified by DNA sequence analysis.

### Results:

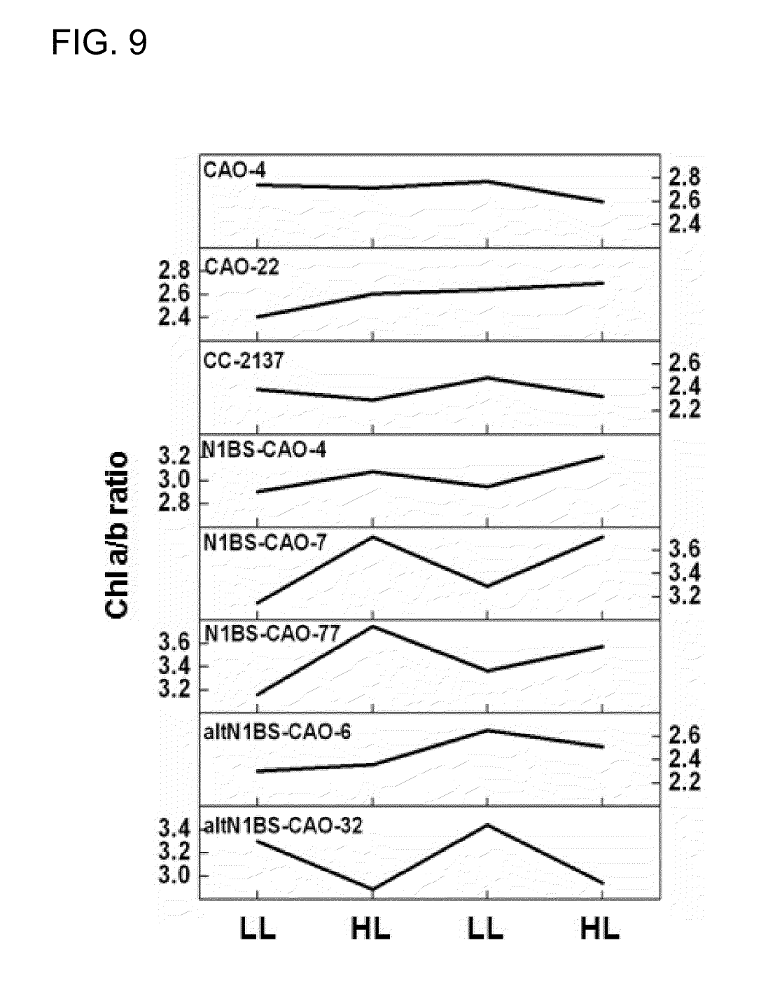

To analyze the effect of the 5′ NAB 1 binding site on the regulation of the CAO gene and Chi b synthesis during photoacclimation, the Chi a/b ratios of the individual transformants were determined by pigment extraction and HPLC analysis of cultures grown at LL (50 [symbol] photons m″² s′¹) or at HL (500 [symbol] photons m″² s″¹) for 6 days. Each strain was inoculated into fresh HS media using a 2% v/v culture inoculum to avoid self-shading and nutrient limitation. Chi a/b ratios were then monitored through two sets of alternating periods of low and high irradiance as shown in FIG. 9.

The complemented wild-type (CAO-4, 22) and CC-2137 strains showed similar trends with slight reductions (<2-6%) in Chi a/b ratios when grown under HL probably due to the effects of photoinhibition (Harper et al., (2004) Photosynth. Res. 79: 149-159). The N1 BSCAO-4, 7 and 77 transgenics on the other hand showed the opposite trend with up to a −16% increase in Chi a/b ratios when grown under HL conditions. This is indicative of a preferential decrease in Chi b synthesis in response to high irradiances due to NAB 1 regulation of CAO. By contrast, the altN1 BS-CAO transgenics showed trends, i.e. a lack of change in chlorophyll b content with changes in light intensity, similar to the complemented wild-type and CC-2137 strains suggesting that NAB 1 binding to the CAO transcript was probably perturbed due to the alterations to the sequence of the binding site

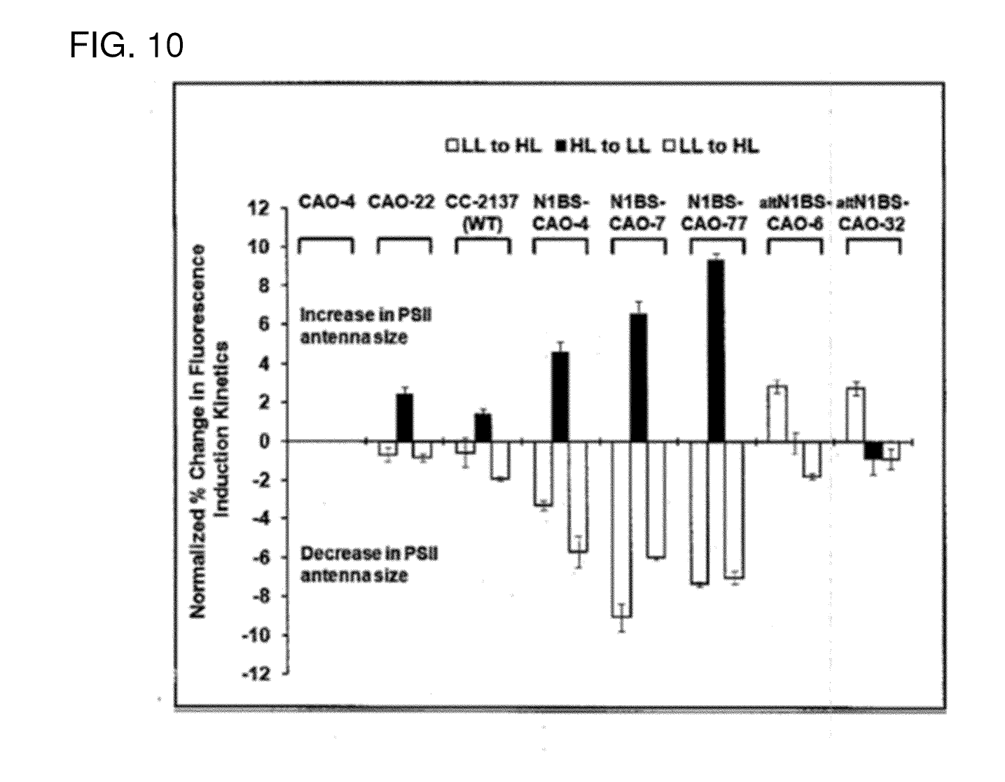

To correlate the changes in Chi a/b ratio to possible alterations in PSII antenna size, test cells were subjected to flash fluorescence induction as described previously. After each light period, the percentage light saturation or reaction center closure was calculated for the transformants at a time point where the complemented wild type strain, CAO-4, achieved 90% saturation. The values obtained for each strain under low and high light were compared to yield a percentage decrease/increase in Chi fluorescence yield. The results in FIG. 10 show reversible changes in Chi fluorescence induction kinetics of up to −10% that were observed after each light cycle in the N 1 BS-CAO transgenics as compared to less than −1-2% change in the CC-2137 wild-type control.

### Conclusion:

The results from these three independent transformants expressing the modified CAO gene confirm that Chi a/b ratios increase under high light acclimation resulting in reduced PSII antenna size. Conversely, a decrease in Chi a/b ratios under conditions of low irradiances are indicative of an increase in PSII antenna size. This interpretation is supported by the observation that flash Chi fluorescence induction kinetics of low light grown cultures exhibited up to −10% increase in the level of light saturated Chi fluorescence compared to the complemented wild-type CAO-4, and a −10% reduction in Chi fluorescence yield relative to 90% light saturation yield for wild type when grown under high light conditions. This light-dependent change in antennae size in the transgenics is substantially greater than that observed in wild-type cells, and consistent with the hypothesis that the system is indeed working as predicted to automatically regulate PSII antenna size in response to ambient light intensity

### Example 5

### Exogenous Agent for Conversion of Carbon Dioxide to Bicarbonate, and Methods for Identifying Such Agents, for Use in Biocompatible Polymers

This example provides examples of exogenous agents (e.g., carbonic anhydrase enzymes) capable of converting carbon dioxide to biocarbonate.

To address the industrial need to have more stable CAs that retains desirable kinetic properties, five mutants of HCA II were constructed using site-specific mutagenesis (see Fisher, Z. et. al., Protein Engineering and Design, pgs. 1-9 (2012)). The three residues changed to investigate stability were selected out of 10 possible mutations discovered using a random mutagenesis approach as described in U.S. Pat. No. 7,521,217 ('217 patent). The mutants were scored for thermal stability after incubating the mutants at elevated and defined temperatures for 2 hours and then measuring residual activity. The results were then expressed as percent residual activity compared with wild-type HCA II. The authors of the patent showed that one mutation at a time had a very modest effect and that they could achieve much higher melting temperatures by combining the mutations. There was no specific order that mattered and that a minimum of three mutations yielded increased percent residual activity after incubation for 2 hours at 10° C. higher than wild type. After careful inspection of the crystal structure of wild-type HCA II, three Leu residues of the 10 mutations were selected for the following reasons: (i) they are all on the surface of HCA II, (ii) they are all hydrophobic Leu residues, (iii) they cluster together in a patch on the surface of the enzyme and (iv) they are sufficiently far away from the active site that we were sure not to disturb the pKa of the proton donor/acceptor during catalysis. The other mutations that contributed to increased thermal stability identified in the '217 patent are Ala65Thr, Phe93Leu, Gln136His/Tyr, Lys153Asn, Leu198Met and Ala247Thr (each amino acid is identified by its three letter code; the mutations are identified by the general formula of [Wild-type Amino Acid]-[Amino Acid Position in the Protein]-[Amino Acid that Replaced the Wild-type Amino Acid]; for example “Ala65thr” represents that the wild-type amino acid Alanine at position 65 of the Human CAII protein was replaced with a Threonine). Our three Leu mutations served as the background to which strategic active site mutations were added to create active HCA II variants with improved stability and kinetics in some cases. Surprisingly, some of the mutants displayed improved proton transfer rates compared with wild type while CO₂ hydration rates were unaffected. To better understand the biophysical effect of thermostabilizing mutations, X-ray structures of the mutants were solved and enzyme kinetics were determined under a variety of possible industrial environmental conditions. These data show that changing hydrophobic surface residues in HCA II to polar/charged ones can improve stability through an electrostatic mechanism. Simultaneously, it is possible to fine tune some of the enzyme kinetic parameters while creating variants with improved thermal stability.

### X-Ray Crystallography and Structural Analysis:

Site-specific mutations of HCA II were made by GenScript. The first mutant, thermostable 1 (TS1) was constructed base on results reported in U.S. Pat. No. 7,521,217 (filed by CO2 Solutions) and contained the following single amino acid substitutions: Leu100His as well as Leu224Ser and Leu240Pro. This triple mutant then served as the background for TS2-TS5. In addition to the starting triple mutations, TS2 also contained Tyr7Phe, TS3 had Tyr7Phe [symbol] Asn62Leu, TS4 had Tyr7Phe [symbol] Asn67Gln and TS5 had six mutations with Tyr7Phe [symbol] Asn62Leu [symbol] Asn67Gln added. The proteins were expressed and purified as described elsewhere (Fisher et al., 2009). After purification the proteins were concentrated and buffer exchanged into 50 mM Tris pH 7.8 using Amicon Ultra concentration devices with a 10 kDa molecular weight cut-off. The proteins were concentrated to 35-50 mg/ml prior to all subsequent experiments and characterizations.

The HCA II variants did not readily crystallize with the usual published conditions of ammonium sulfate or sodium citrate for HCA II (Fisher et al., 2007a,b). Therefore the Gryphon robotic drop-setter from Art Robbins Instruments was used to set up vapor diffusion sitting drops against different commercial screens. Diffraction quality crystals were obtained within a week from Hampton Screen 1, condition #6 (0.2 M magnesium chloride heptahydrate, 0.1 M Tris pH 8.5 and 30% Peg 4000) using a sample concentration of 50 mg/ml. The crystals were flash-cooled by rapidly placing them in cold gas stream with no added cryoprotectant. X-ray diffraction data at 100 K were collected on an RAXIS IV⁺⁺ using an in-house rotating Cu anode HU-H3R. The frames were collected with 18 oscillation steps and 5 min per exposure. The crystal to detector distance was 80 mm. All crystals diffracted between 1.56 and 2.0 Å resolution. Data processing and reduction were done with either d*TREK or the HKL2000 suite of programs (Otwinowski and Minor, 1997; Pflugrath, 1999). The starting model was derived from protein data bank (PDB) accession number 2ili with all the waters and Zn removed, and with the mutated residues changed to Gly (Fisher et al., 2007b). All the structures were refined using PHENIX and manual inspection and model building was done using Coot (Emsley and Cowtan, 2004; Adams et al., 2010). The models for all of the variants were refined consistently in that there were no deleted or truncated residues and water molecules with a B-factor <40A Å were removed. All crystallographic figures were generated using PyMOL (DeLano, 2002). Experimental data and structural coordination have been deposited with the Protein Data Bank and have the following accession numbers: TS1=3V3F, TS2=3V3G, TS3=3V3H, TS4=3V31 and TS5=3V3J.

Results: All variants crystallized in the orthorhombic P2₁2₁2₁ space group and were highly isomorphous with approximate unit cell dimensions a=42, b=72 and c=75 Å and diffracted between 1.56 and 2.0 Å resolution. All models refined to R_{cryst} and R_{free} between ˜16-19 and 20-25%, respectively.

Leu100, Leu224 and Leu240 are all between 8 and 14 Å from each other (Ca—Ca distance) and form a small hydrophobic patch on the surface of HCAII. This patch is at least 20 Å away from the zinc active site. The mutated residues are located within the interface of two crystallographic symmetry-related chains that might contribute to different crystal packing compared with wild-type HCA II. Based on a comparison of the mutant and wild-type X-ray crystal structures it is clear that changing these hydrophobic residues to polar residues results in increased stability through differences in enthalpic contributions with the gain of H-bonds and favorable. This decrease in surface hydrophobicity likely contributes to the increased solubility and different crystallization conditions required to crystallize the mutants.

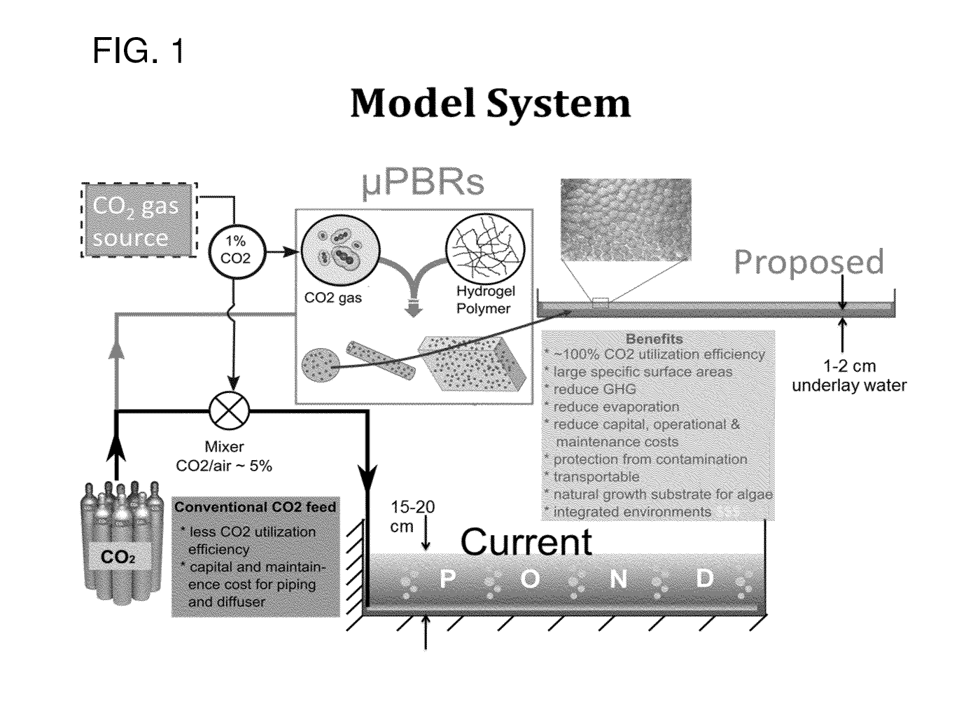

Leu100His accommodates weak H-bonds (3.0-3.4 Å) with the backbone amide of Gly102 and the side chain of Gln103 (FIG. 1b). The average B-factors of all atoms in the loop consisting of residue 97-104 is ˜15 Å² and are similar compared with wild-type structures also determined at 100 K. This supports the notion that a gain in H-bonding at position 100, and not thermal movement, dominates the observed increased stability. The presence of a small hydrophobic residue at this position is highly conserved in several homologous human CAs.

Leu224Ser is observed in a dual conformation making H-bond contacts to either backbone amides of residues Ser220 or Glu221, as well as to a water molecule that is also coordinated to the guanido group of Arg227. A comparison of the average B-factors for all atoms from residue 223-225 is interesting: for wild type it is ˜12 Å² and 19 and 26 Å² for TS2 and TS4, respectively. This implies an increased thermal fluctuation in the mutant compared with wild type that is also reflected by the disorder of Ser224. Leu at this position is completely conserved in several other human CAs such as isoforms IV and XII, but interestingly is a Ser in the mitochondrial form, CA V.

Leu240Pro creates a solvent accessible, hydrophilic pocket that allows for the ordering of two water molecules to make H-bonds with the backbone carbonyls of residues Lys228 and Leu229. The introduction of a Pro at position 240, which sits at the end of a surface loop, may be expected to cause a reduction in the loop flexibility. However, similar to the mutations introduced at positions 100 and 224, changing Leu 240 to Pro appears to increase the average B-factor of the loop from ˜12 to 35 Å². Having a Leu at position 240 does not seem conserved among different human isoform, but there are Pro residues in three human CAs: I, III and XII. This is interesting as the phi/psi space that Pro can occupy is narrowly defined as between ˜−60° C. and either ˜30° C. or +135° C. These angles are virtually identical in the wild-type and TS1-5 mutants.

Altered loop conformations at residues Val37-Ser50, Phe70-Lys80 and Lys225-Leu240 are observed in these mutants relative to the wild-type structure. The Cα backbone trace of the Phe70-Lys80 loop is displaced by up to 3.3 Å compared with wild type with the most dramatic effect arising from the movement of Lys76 that now makes an H-bond with Asp71 with Gln74. These alternative loop conformations have been observed with HCA II: inhibitor structures solved in the orthorhombic space group as well as for other HCA II structures with mutations in or near the opening of the active site (Ippolito et al., 1995; Lloyd et al., 2005). These displaced surface loops are most likely a direct consequence of the crystal packing forces.

As a result of the crystallization conditions used for the TS mutants of HCA II, a single Cl2 is seen along the surface within H-bonding distance of the amide groups of Gln158 and Lys225 for TS1-4. In the TS5 structure Lys225 is not in position to H-bond with Cl2, which allows Glu158 to be present at the Cl2 binding site. As a result, a water molecule is seen in this position instead of the Cl2 ion. It is not obvious why Lys225 occupies this unique conformation in TS5 compared with TS1-4. In addition, previous studies of HCA II in an orthorhombic space group report a Zn coordinated to His4 located near the opening of the active site cavity (Lloyd et al., 2005; PDB: 2X7S). Interestingly, in our TS2 structure (PDB: 3V3G), there is similar density observed in this region. However, the N1 group of residue His3 is coordinating to the observed density along with possible interactions from His64, the symmetry-related His36 residue and a water molecule. Attempts at placing Zn in this density resulted in increased B-factors (80 Å²) in addition to appearance of negative density in the Fo-Fc map. Owing to the uncertainty at this position, a water molecule was built and refined (B-factor, 20 Å²). Nevertheless, it is worth noting that the possible Zn coordination site at the N-terminus is very similar to the canonical Zn-His arrangement found in the active site.

There are no significant active site differences between wild type and TS1. The waters and positions of the residues are essentially conserved, except that His64 in TS1 is observed in the ‘outward’ conformation compared with wild-type HCA II, where an ‘in’ and ‘out’ conformation is observed (Nair and Christianson, 1991; Fisher et al., 2005). In TS2 where Tyr7 is replaced with a Phe, the solvent W3a is displaced as compared with wild-type HCA II and can no longer participate in the H-bonding network in the active site. This is consistent with the results published previously (Fisher et al., 2007a,b). The rest of the waters and residues are the same as in wild type, except that W2 and H64 now appear to engage in an H-bond. In TS3 the solvent W3b has moved but is still H-bonded to Asn67 and W2. Similarly to TS2, the introduction of a hydrophobic residue causes displacement of active site water molecules. In TS4 Gln67 mostly maintains a similar H-bond to W3b while in TS5 it is somewhat disordered and observed in two conformations. This displaces W3b completely and one conformer of Gln67 is observed to directly participate in a weak H-bond (O . . . O distance 3.4 Å) to W2. These results are expected based on previous structure-function studies of HCA II and indicate that the presence of the surface Leu mutations do not affect the active site structure in the TS mutants (Fisher et al., 2007a,b; Mikulski et al., 2011).

### Catalytic Activity:

The ¹⁸O exchange method is based on mass spectrometric measurements using a membrane inlet of the depletion ¹⁸O from CO₂ (Silverman, 1982). The isotopic content of CO₂ in solution is measured when it passes across a membrane and into an Extrel EXM-200 mass spectrometer. The measured variable is the atom fraction of 180 in CO₂. The first step of catalysis has a probability of reversibly labeling the Zn-bound OH⁻ with ¹⁸O (reaction (3)). During the next step the ¹⁸OH⁻ can be protonated and results in the release of H₂¹⁸O to the bulk solvent where it is essentially infinitely diluted by H₂¹⁶O (reaction (4)). In this process, His64 acts as a proton shuttle (Tu et al., 1989):

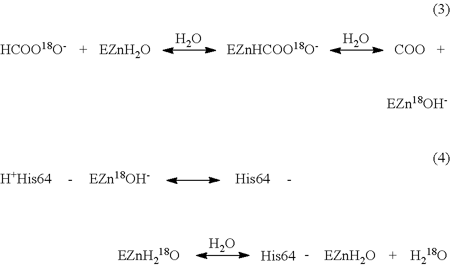

The ¹⁸O-exchange method obtains two different rates at chemical equilibrium (Silverman, 1982): R₁, which is the rate of exchange of CO₂ and HCO₃⁻ (reaction (5)) and R_{H2O}, which is the rate of release of H₂¹⁸O from the enzyme. In reaction (5), k_{cat}^{ex} is the rate constant for maximal conversion between substrate and product while K_{eff}^{s} is the effective binding constant of the substrate ([S] is the concentration); [S] can be either CO₂ or HCO₃⁻ depending on the direction of the reaction. The ratio expressed in reaction (5) of k_{cat}^{ex}/K_{eff}^{s} is in principle the same as k_{cat}/K_{M} obtained under steady-state conditions:

R₁/[E]=k_{cat}^{ex}[S]/(K_{eff}^{s}+[S]) (5)

In the second part of catalysis the rate R_{H2O} is the part of ¹⁸O exchange that is dependent on the rate of proton transfer from His64 to the labeled enzyme-bound OH⁻ (i.e. in the dehydration direction) (Tu et al., 1989). Reaction (6) shows the relationship between k_{B}, the rate constant for proton transfer to Zn-bound OH⁻ and (Kₐ)_{donor} and (Kₐ)_{ZnH2O} that are the ionization constants for the proton donor and Zn-bound water, respectively:

k_{B}^{obs}=k_{B}/[[1+k_{a(donor)}/[H⁺]][1+[H⁺]/(Kₐ)/Zn_{H20}]] (6)

Except for the temperature dependence studies, all enzyme kinetic measurements were done at 25° C. in the absence of buffer using a total substrate concentration (all species of CO₂) of 25 mM. The temperature dependence studies used 10 mM total species of CO₂. Kinetic constants and ionization constants shown in reactions (5) and (6) were determined through nonlinear least squares methods (Enzfitter, Biosoft).

### Results:

The pH profiles were determined for R₁, the rate of catalyzed interconversion of CO₂ and bicarbonate (reaction (5) and R_{H2O}, the rate of dissociation of H₂¹⁸O from the active site (reaction (6), using the ¹⁸O-exchange method. The background mutations (Leu100His, Leu224Ser and Leu240Pro) did not significantly affect the rate of CO₂ hydration reflected through=k_{cat}^{ex}/K_{eff}^{s} (Table 3) or R₁/[E]. Moreover, the replacements at positions 7, 62 and 67 also caused no significant changes in these measures of the first stage of catalysis (reaction (3)). This result was expected since the surface Leu mutations and the amino acid replacements in TS1-5 are sufficiently far from the catalytic Zn to avoid structural and electrostatic disruptions of the reaction of Zn-bound OH⁻ with substrate.

However, there are interesting differences in the rate constants R_{H2O}/[E] and the rate constant for proton transfer in catalysis k_{B} (Table 3). The rate constant k_{B} measures in large part the proton transfer along an ordered water structure between His64 and the Zn-bound OH⁻ in the dehydration direction and is determined from the bell-shaped pH profiles such as observed with wild-type HCA II (Silverman, 1982; Tu et al., 1989; Silverman and McKenna, 2007). These results show that the background replacements of surface Leu residues in TS1 do not negatively affect R₁ or k_{B} compared with wild type (Table 3), consistent with their location far from the active site and proton transfer pathway (Silverman and McKenna, 2007). However, combining the background mutations in TS1 with specific active site changes at positions 7 and 67 caused an unexpected, albeit modest, boost in proton transfer activity (bold numbers, Table 3). Overall however, the proton transfer efficiency of the remaining variants can be understood in terms of the results for the corresponding variants with single amino acid replacements. The value of k_{B} for Tyr7Phe HCA II is increased ˜5-fold compared with wild type (Table 3, Fisher et al., 2007a, b); accordingly, it was expected that the variant TS2 has a higher value of k_{B} compared mutants, we concluded that the displacement of W3a and the loss of the hydroxyl group at position 7 led to changes in pKₐ for proton donor/acceptor groups. In addition to the electrostatic and related pKₐ changes that occurred also produced a shorter, unbranched chain of hydrogen-bonded waters that connect ZW to the proton shuttling residues His64. These electrostatic changes and unbranched water network boost the proton transfer activity of HCA II Tyr7Phe mutants (Fisher et al., 2007b). As reported in Table 3, the values of k_{B} for TS2 and TS4 are even better than for Tyr7Phe alone at 5.6 and 4.9, respectively. The value of k_{B} for Asn67Gln HCA II is increased ˜2-fold compared with wild type (Mikulski et al., unpublished), and for Asn62Leu HCA II the value of k_{B} is decreased 8-fold. These factors then influence the values of k_{B} shown in Table 3; for example, the two variants (TS3 and TS5) containing Asn62Leu have low values of R_{H2O}/[E]. It is important to point out that from an industrial and applications point of view, it is the value of k_{cat}^{ex}/K_{eff}^{s} that is significant for low concentrations of CO₂, say, 10 mM. The values of kB measuring proton transfer come into significance when HCA II is under maximal velocity conditions for concentrations of CO₂>10 mM.

TABLE 3

| Enzyme | k^{ex}_{cat}/K^{s}_{eff} (μM⁻¹s⁻¹) | k_{B}^{a} (μs⁻¹) |
|---|---|---|
| Wild type | 120 | 0.8 |
| Y7F^{b} | 120 | 3.9 |
| TS1 | 85 | 1.3 |
| TS2 | 110 | 5.6 |
| TS3 | 88 | ~0.1 |
| TS4 | 94 | 4.9 |
| TS5 | 110 | ~0.1 |

^{a}The standard errors are in the range of 10-20%.
^{b}From Fisher et al. (2007a, b). Data at 10° C.

### Chemical and Thermal Stability:

Enzyme activity was measured while increasing amounts of urea up to 8 M in 1 M increments. The solutions contained urea, 25 mM of all species of CO₂ and 100 mM 4-(2-hydroxyethyl)-1-piper-azineethane sulfonic acid (HEPES) at pH 7.6 and 25° C. An enzyme was added to the reaction vessel and catalytic ¹⁸O exchange activity was measured as R₁/[E] (reaction 5) over a period of up to 5 min. Enzyme activity as R₁/[E] was also measured at temperatures from 10 to 70° C. in the similar solutions (100 mM HEPES and 10 mM total substrate) at pH 7.6. After the reaction solution was equilibrated to each different temperature, a small sample of enzyme (at 0.1-0.2% of reaction volume) was added. Measurements of ¹⁸O content of CO₂ were made over the following 1-5 min.

Differential scanning calorimetry (DSC) experiments were performed using a VP-DSC calorimeter (Microcal, Inc., North Hampton, Mass., USA) with a cell volume of ˜0.5 ml. All wild type, Tyr7Phe and TS mutant HCA II samples were buffered in 50 mM Tris-HCl, pH 7.8, at protein concentrations of ˜30 μM. The samples were degassed while stirring for 1 h before data collection. The DSC scans were collected from 20 to 100° C. with a scan rate of 60° C./h. The calorimetric enthalpies of unfolding were calculated by integrating the area under the peaks in the thermograms after adjusting the pre- and post-transition baselines. The thermograms were fit to a two-state reversible unfolding model to obtain van't Hoff enthalpies of unfolding. The melting temperature (T_{M}) values of the HCA II samples were obtained from the midpoints on the DSC curves, indicating a two-state transition. All samples were measured in triplicate with a buffer baseline subtracted.

### Results:

To test the stability of the variants against denaturation through thermal and chemical means, a series of experiments was carried out by DSC and by measuring the rate of catalyzed CO₂/HCO₃⁻ interconversion. First, differential scanning calorimetry scans were measured for wild type, Tyr7Phe mutant and each of the TS HCA II variants. The melting temperatures or major unfolding transitions (T_{M}) for each of the variants occurred at distinct peaks in the thermograms. The average peak values (with standard deviations shown in parentheses) from at least three runs are given in Table II. The CD data were consistent with the DSC data for each variant but showed higher temperatures of unfolding (˜2° C.), probably reflecting the retention of secondary structure elements after initial melting (data not shown). Wild-type HCA II had a T_{M} of ˜58° C. under these experimental conditions and introducing the three background mutations present in TS1 (Leu100His, Leu224Ser and Leu240Pro) increased the T_{M} to 65° C. Introducing the Tyr7Phe mutation to increase proton transfer activity had a destabilizing effect, reducing the T_{M} to 53° C. (Fisher et al., 2007a,b; Mikulski et al., 2011) while adding the background mutations ‘rescues’ the stability by increasing it to ˜61° C. for TS2. Addition of Asn67Glu to an active site containing Tyr7Phe stabilizes HCA II almost back to wild-type levels while also displaying high catalytic efficiency. The remaining variants contain different active site mutations and have varying effects on stability. Among all of the variants, TS1, TS2 and TS4 have values of T_{M} that are significantly higher than wild-type HCA II. This is very encouraging as these variants have not only different stabilities but also somewhat different kinetic profiles compared with wild type that make them interesting from an industrial point of view (Fisher et al., 2007a,b; Zheng et al., 2008; Mikulski et al., 2011).

The rate constant R₁/[E] was determined while increasing temperature from 10 to 70° C. These are rough estimates of the thermal inactivation temperature with measurements made in intervals of 5° C. at the higher temperatures the purpose of which was to determine whether there was inactivation of catalysis at a temperature less than the major unfolding determined by DSC. What is important here is that there appears no inactivation of catalysis at temperatures less than the T_{M} determined by DSC, and that the more accurate major unfolding transition determined as T_{M} by DSC most likely also measures the thermal stability of catalysis.

The chemical stabilities of the mutants were compared with wild-type HCA II increasing urea concentration up to 8 M in 1 M increments while measuring the kinetic constant R₁/[E]. The Leu surface mutations had no significant effect on enzyme ability to withstand denaturation by urea under these conditions. The addition of 4 M urea led to <10% relative activity compared to no urea for all variants, including wild type. This observation can be partly explained by urea denaturation thought to occur through unfolding of the hydrophobic core of the protein. It has also been pointed out that urea can interact with the protein through electrostatic, van der Waals interactions, and indirectly through the disruption of water structure (Samiotakis et al., 2010; Wang et al., 2011). Since the mutations reported here were all on the surface and active site of the protein (that is, not in the core), the denaturation resulted in similar effects in the wild-type and mutant enzymes. Previous studies aimed at the relationship between chemical and thermal denaturation have also revealed discrepancies similar to the ones we have observed here for apoazurin, cytochrome c and apoflavodoxin (Wang et al., 2011). Our metric for chemical denaturation involved activity assays only and not DSC. These techniques measure different properties of the protein and it is possible that HCA II is still fairly well folded up to 4 M urea but that the assay conditions have been compromised. CO₂ hydration and subsequent proton transfer is strongly dependent on the water structure in the active site and this can easily be disrupted by excess urea (Silverman and McKenna, 2007). The relationship between thermal and chemical denaturation is complex and appears to be unique to different proteins, it is prudent to determine these values empirically for each system under study.

From a structural perspective the rationale for how these mutations confer stability is not intuitive. In contrast to the B FIT approach, an increase in the thermal fluctuation of residues at positions 224 and 240 is observed with a concomitant increase in thermal stability (Reetz et al., 2006). This probably reflects the dominant effect of the gain in H-bonding and hydrophilicity over flexibility on the surface of HCA II. The underlying principle of thermal stability as a change in surface electrostatics reported here for HCA II is consistent with several other successful studies on diverse enzymes such as ubiquitin, acylphosphatase and α-lactalbumin (Permyakov et al., 2005; Strickler et al., 2006; Jochens et al., 2010).

### Conclusion:

Thermal stability of HCA II was enhanced by strategic replacement of amino acids on the surface of the enzyme. Moreover, these replacements had no significant effect on the active site structure and no effect on the catalytic rate of CO₂ hydration and HCO₃⁻ dehydration. Single amino acid replacements that were previously found to enhance catalysis were also effective in enhancing catalysis in variants with these surface changes. The net result was a variant of HCA II (TS4) with thermal stability enhanced by ˜6° C. and with maximal proton transfer enhanced ˜6-fold compared with wild-type HCA II. Further analysis of the surface of HCA II shows that there are other areas to target using this approach. Phe20 and Leu57 are located on the surface also and could be modeled to engage in H-bonds with surrounding residues. Leu204 and Val135 are very close together on the surface and could be mutated so that changes at these positions make a salt bridge or H-bond to each other. The initial results reported here shed light on the underlying biophysical principle, which is removing surface hydrophobic residues and replacing them with polar or hydrophilic residues leads to a gain in H-bonding interaction and this results in increased thermal stability.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### REFERENCES

Adams et al. (2010) Acta Crystallogr., D66, 213-221.
Avvaru et al. (2009) Biochemistry, 48, 7365-7372.
DeLano, (2002) The PyMOL Molecular Graphics System. DeLano Scientific, San Carlos, Calif., http://www.pymol.org
Domsic et al. (2008) J. Biol. Chem., 283, 30766-30771.
Emsley & Cowtan (2004) Acta Crystallogr., D60, 2126-2132.
Filikov et al. (2002) Protein Sci., 11, 1452-1461
Fisher et al. (2005) Biochemistry, 44, 1097-1105.
Fisher et al. (2009) Acta Crystallogr., F65, 495-498.
Fisher et al. (2007a) Biochemistry, 46, 3803-3813.
Fisher et al. (2007b) Biochemistry, 46, 2930-2937.
Fulke et al. (2010) Bioresour. Technol., 101, 8473-8476.
Huang et al. (2002) Biochemistry, 41, 7628-7635.
Ippolito et al. (1995) Protein Eng., 8, 975-980.
Jochens et al. (2010) Protein Eng. Des. Sel., 23, 903-909.
Kanbar & Ozdemir (2010) Biotechnol. Prog., 26, 1474-1480.
Krebs & Fierke (1993) J. Biol. Chem., 268, 948-954.
Krebs et al. (1993) J. Biol. Chem., 268, 27458-27466.
Lloyd et al. (2005) J. Med. Chem., 385, 715-720.
Mikulski et al. (2011) Arch. Biochem. Biophys., 506, 181-187.
Mrabet et al. (1992) Biochemistry, 31, 2239-2253.
Nair & Christianson (1991) J. Am. Chem. Soc., 113, 9455-9458.
Nair & Christianson (1993) Biochemistry, 32, 4506-4514.
Otwinowski & Minor (1997) Methods Enzymol., 276, 307-326.
Permyakov et al. (2005) Protein Eng. Des. Sel., 18, 425-433.
Pflugrath (1999) Acta Crystallogr., D55, 1718-1725.
Potapov et al. (2009) Protein Eng. Des. Sel., 22, 553-560.
Ramanan et al. (2010) Bioresour. Technol., 101, 2616-2622.
Reetz et al. (2006) Angew. Chem., 118, 7909-7915.
Samiotakis et al. (2010) J. Chem. Phys., 132, 175101.
Saville & Lalonde, (2011) Curr. Opin. Biotech., 22, 1-6.
Strickler et al. (2006) Biochemistry, 45, 2761-2766.
Silverman, (1982) Methods Enzymol., 87, 732-752.
Silverman & Lindskog (1988) Acc. Chem. Res., 21, 30-36.
Silverman & McKenna, (2007) Acc. Chem. Res., 40, 669-675.
Tu et al. (1989) Biochemistry, 28, 7913-7918.
Vinoba et al. (2011) Langmuir, 27, 6227-6234.
Wang et al. (2011) J. Chem. Phys., 135, 175102.
Zheng et al. (2008) Biochemistry, 47, 12028-12036.

1. A composition comprising a biocompatible polymer bead having inorganic carbon and algae.

2. The composition of claim 1, wherein the biocompatible polymer is a homopolymer or heteropolymer or combination thereof.

3. The composition of claim 1, wherein the biocompatible polymer comprises a polysaccharide.

4. The composition of claim 1, wherein the biocompatible polymer is a hydrogel foam.

5. The composition of claim 1, wherein the biocompatible polymer comprises cross-linked monomers selected from the group consisting or organic monomers, inorganic monomers and combinations thereof.

6. The composition of claim 1, wherein the biocompatible polymer comprises cross-linked monomers selected from the group consisting of alginate, agar, carrageenins, cellulose, a combination of silicone and/or siloxanes with polyacrlymide and combinations thereof.

7. The composition of claim 1, wherein the monomers of the biocompatible polymer are cross-linked with a multivalent cation.

8. The composition of claim 6, wherein the multivalent cation is selected from the group consisting of a metal cation, an amine, an amino acid derivative, a water-miscible organic solvent and combinations thereof.

9. The composition of claim 8, wherein the metal cation is selected from the group consisting of calcium, magnesium, iron, copper, zinc, mangenses, potassium, sodium, ammonia, biocompatible Lewis acid metals and combinations thereof.

10. The composition of claim 1, wherein the monomers of the biocompatible polymer are cross-linked with an anion.

11. The composition of claim 10, wherein the anion is selected from the group consisting of phosphate, selenate, nitrate, chloride sulfate and combinations thereof.

12. The composition of claim 1, wherein the volume of inorganic carbon in the biocompatible polymer is up to 60%.

13. The composition of claim 1, wherein the inorganic carbon is selected from the group consisting of carbon dioxide, carbonic acid, bicarbonate anion, carbonate and a combination thereof.

14. The composition of claim 1, wherein the inorganic carbon forms pockets in the biocompatible polymer having an average diameter of from 0.5 nm to about 10 nm.

15. The composition of claim 1, wherein the algae are modified to have increased light utilization efficiency compared to wild-type algae of the same strain.

16. The composition of claim 1, wherein the algae have a photosynthetic rate that is higher than wild-type algae of the same strain at saturating light.

17. The composition of claim 1, wherein the algae have at least 10% greater biomass than wild-type algae of the same strain.

18. The composition of claim 1, wherein the peripheral light harvesting antenna size of photosystem II of the algae is smaller than the peripheral light harvesting antenna size of photosystem II of wild-type algae of the same strain.

19. The composition of claim 1, wherein the ratio of chlorophyll a to chlorophyll b of green algae (Chlorophyta) is greater than the ratio of chlorophyll a to chlorophyll b of wild-type algae of the same strain.

20. The composition of claim 1, wherein the ratio of chlorophyll a to chlorophyll b of the algae is from about 3 to about 7.

21. The composition of claim 1, wherein the chlorophyll b content of the algae is reduced by an RNAi mechanism.

22. The composition of claim 1, wherein the algae comprise a siRNA that targets the chlorophyllide an oxygenase (CAO) gene.

23. The composition of claim 1, wherein the algae's endogenous CAO gene levels are reduced compared to the CAO gene levels of a wild-type algae of the same strain.

24. The composition of claim 23, wherein the translation activity of the CAO gene is reduced or inhibited with a nucleic acid binding protein 1 (NAB1).

25. The composition of claim 1, wherein the algae is a transgenic algae expressing a protein comprising the amino acid sequence selected from the group consisting of SEQ ID NOs: 1, 2, 3 and combination thereof.

26. The composition of claim 1, wherein the strain of algae is selected from the group consisting of Chlamydomonas reinhardtii, Chlorella sp., Synechocystis sp., Synechococcus, Anabaena sp., Cyclotella, Phaeodactylum sp., Crypthicodineum sp., Schizochytridum sp., Haematococcus sp., Arthrospira (Spirulina) sp, Dunaliella sp. and combination thereof.

27. The composition of claim 1, wherein the biocompatible polymer further comprises a light frequency-shifting agent.

28. The composition of claim 27, wherein the light frequency-shifting agent is red light emitting.

29. The composition of claim 27, wherein the light frequency-shifting agent absorbs light comprising the light spectrum of from ultraviolet to green light and emits light comprising red light.

30. The composition of claim 27, wherein the light frequency-shifting agent is selected from the group consisting of a quantum dot, a fluorescent protein and a combination thereof.

31. The composition of claim 27, wherein the association between the light frequency-shifting agent and the biocompatible polymer is selected from the group consisting of a covalent bond, non-bonded interactions and a combination thereof.

32. The composition of claim 30, wherein the light frequency-shifting agent is a colloidal nanocrystal quantum dot.

33. The composition of claim 32, wherein the colloidal nanocrystal quantum dot comprises an inner core having an average diameter of at least 1.5 nm and an outer shell, wherein the outer shell comprises multiple monolayers of an inorganic material.

34. The composition of claim 32, wherein the colloidal nanocrystal quantum dot outer shell comprises at least four monolayers of inorganic material.

35. The composition of claim 32, wherein the colloidal nanocrystal quantum dot outer shell comprises from about four to twenty monolayers of inorganic material.

36. The composition of claim 32, wherein the colloidal nanocrystal quantum dot exhibits an effective Stokes shift of at least 75 nm.

37. The composition of claim 32, wherein the colloidal nanocrystal quantum dot inner core comprises material selected from the group consisting of CuInS2, Zn3P2, GaP, GaAs, GaSb, InP, InAs, InSb, ZnS, ZnSe, ZnTe, CdSe, CdS, CdTe, PbS, PbSe, PbTe, and combinations thereof.

38. The composition of claim 32, wherein the colloidal nanocrystal quantum dot outer shell comprises material selected from the group consisting of ZnS, ZnSe, ZnTe, CdS, CdSe, CuGaS2, GaP, Cu20, AlP, AlAs, GaS, SnS2 and combinations thereof.

39. The composition of claim 32, wherein the colloidal nanocrystal quantum dot inner core and outer shell comprise, respectively, CuInS2 and ZnS, or CuInS2 and ZnSe, or InP and ZnS, or InP and ZnSe, or Zn3P2 and ZnS.

40. The composition of claim 30, wherein the light frequency-shifting agent is a fluorescent protein.

41. The composition of claim 30, wherein the fluorescent protein absorbs light comprising blue light and emits light comprising red light.

42. The composition of claim 30, wherein the fluorescent protein is a fusion protein of a green fluorescent protein (GFP) and a red fluorescent protein (RFP), wherein the fusion protein absorbs light comprising blue light and emits light comprising red light.

43. The composition of claim 1, wherein the biocompatible polymer further comprises an exogenous agent that is capable of converting carbon dioxide to bicarbonate.

44. The composition of claim 43, wherein the association between the exogenous agent and the biocompatible polymer is selected from the group consisting of a covalent bond, non-bonded interactions and a combination thereof

45. The composition of claim 43, wherein the exogenous agent is a carbonic anhydrase enzyme.

46. The composition of claim 45, wherein the amino acid sequence of the carbonic anhydrase enzyme is selected from the group consisting of SEQ ID NOs: 1, 2, 3 and a combination thereof.
